SEQUENCE LISTING (30 sequences)

SEQ ID NO: 1 - DNA, 1200 bp, Porphyromonas gingivalis
atgagattctctgccattattatcgctttgattgtgatgctgcctgctgtgcttagcgggcagcattattattccatggcgggagagcgaccggagacggacagcattcgtccgaacgaactctcggcatcgatccgaagtacgcttttctttcggaacaatgaatacaatgcacgttcggtcaaaggttatacgttgccgggtgcacgggtttccgcttttgcctcttactcgctgccggcagcacatggtgtgaagctttcgctcggagtatctaccctgaactactggggggcaagtcgctatccggccggtatcgcttattccgatttaccttattggacggactataacgactatgtacgcttgcgtatcctgccttatgtacaggccatgctgaagccgacggccacgactgctctcatgctgggcaatatagccggtggtacggctcacggactgatcgaaccgatctacaatcctgagttggatttgacggctgatcctgaagccggtgtgcaatttcggggtgattggacacgtttccgaatggatgtttgggtcaattggatgagcatgattttcaaaaatgacaatcatcaggagtcgtttgtctttggcttgtccactacttccaaattgttatcgggtgaaggcaaatggcgactcgaactgcccttgcaggctattgccacgcatcgcggcggggaatacaactgggcgcagcaggataccgtgcatacatgggtcaatggagctgtcggacttaagctttcgtattgccctcgtaccgacaaacccatgcagatttggggatctgcttatggtgtggcagccttgtcaagcggaggatactttccttacgaaagagggtggggcggttatctttctctcggaatggacttggagcacttcgcttttcgtaccgactattggtacggcaggcattacgtttctccctttgctgcacctttcgccaattccctgacgtatgacaaacagcctcttacgaacggttggggcgattatattcgtctctatgccgactattcgtggcggatggcacgaagtgtttcgttggcggctgttgctcgggtatggttccagccttcggatcgttttgcgatgagccacgccttggaactgacgatgcgtatcgatcccaatttcccgatagcttttctgaaaggcaatcattga

SEQ ID NO: 2 - DNA, 1200 bp, Porphyromonas gingivalis
atgagattctctgccattattatcgctttgattgtgatgctgcctgctgtgcttagcgggcagcattattattccatggcgggagagcgaccggagacggacagcattcgtccgaacgaactctcggcatcgatccgaggtacgcttttctttcggaacaatgaatacaatgcacgttcggtcaaaggttatacgttgccgggtgcacgggtttccgcttttgcctcttactcgctgccggcagcacatggtgtgaagctttcgctcggagtatctaccctgaactactggggggcaagtcgctatccggccggtatcgcttattccgatttaccttattggacggactataacgactatgtacgcttgcgtatcctgccttatgtacaggccatgctgaagccgacggccacgactgctctcatgctgggcaatatagccggtggtacggctcacggactgatcgaaccgatctacaatcctgagttggatttgacggctgatcctgaagccggtgtgcaatttcggggtgattggacacgtttccgaatggatgtttgggtcaattggatgagcatgattttcaaaaatgacaatcatcaggagtcgtttgtctttggcttgtccactacttccaaattgttatcgggtgaaggcaaatggcgactcgaactgcccttgcaggctattgccacgcatcgcggcggggaatacaactgggcgcagcaggataccgtgcatacatgggtcaatggagctgtcggacttaagctttcgtattgccctcgtaccgacaaacccatgcagatttggggatctgcttatggtgtggcagccttgtcaagcggaggatactttccttacgaaagagggtggggcggttatctttctctcggaatggacttggagcacttcgcttttcgtaccgactattggtacggcaggcattacgtttctccctttgctgcacctttcgccaattccctgacgtatgacaaacagcctcttacgaacggttggggcgattatattcgtctctatgccgactattcgtggcggatggcacgaagtgtttcgttggcggctgttgctcgggtatggttccagccttcggatcgttttgcgatgagccacgccttggaactgacgatgcgtatcgatcccaatttcccgatagcttttctgaaaggcaatcattga

SEQ ID NO: 3 - protein, 399 aa, Porphyromonas gingivalis
MRFSAIIIALIVMLPAVLSGQHYYSMAGERPETDSIRPNELSASIRSTLFFRNNEYNARSVKGYTLPGARVSAFASYSLPAAHGVKLSLGVSTLNYWGASRYPAGIAYSDLPYWTDYNDYVRLRILPYVQAMLKPTATTALMLGNIAGGTAHGLIEPIYNPELDLTADPEAGVQFRGDWTRFRMDVWVNWMSMIFKNDNHQESFVFGLSTTSKLLSGEGKWRLELPLQAIATHRGGEYNWAQQDTVHTWVNGAVGLKLSYCPRTDKPMQIWGSAYGVAALSSGGYFPYERGWGGYLSLGMDLEHFAFRTDYWYGRHYVSPFAAPFANSLTYDKQPLTNGWGDYIRLYADYSWRMARSVSLAAVARVWFQPSDRFAMSHALELTMRIDPNFPIAFLKGNH

SEQ ID NO: 4 - protein, 399 aa, Porphyromonas gingivalis
MRFSAIIIALIVMLPAVLSGQHYYSMAGERPETDSIRPNELSASIRGTLFFRNNEYNARSVKGYTLPGARVSAFASYSLPAAHGVKLSLGVSTLNYWGASRYPAGIAYSDLPYWTDYNDYVRLRILPYVQAMLKPTATTALMLGNIAGGTAHGLIEPIYNPELDLTADPEAGVQFRGDWTRFRMDVWVNWMSMIFKNDNHQESFVFGLSTTSKLLSGEGKWRLELPLQAIATHRGGEYNWAQQDTVHTWVNGAVGLKLSYCPRTDKPMQIWGSAYGVAALSSGGYFPYERGWGGYLSLGMDLEHFAFRTDYWYGRHYVSPFAAPFANSLTYDKQPLTNGWGDYIRLYADYSWRMARSVSLAAVARVWFQPSDRFAMSHALELTMRIDPNFPIAFLKGNH

SEQ ID NO: 5 - DNA, 11153 bp, Artificial Sequence
  Synthetic
tctaaatttaaatataaacaacgaattatctccttaacgtacgttttcgttccattggccctcaaaccccgttatatacattcatgtccatttatgtaaaaaatcctgctgaccttgtttatgtcttgtcagtcaccatttgcaaaaccatatttgaccctcaaagaggctgaatttgataagcaacttgctacatactcataataaggagctaaatagaacacgaatgggaaatactcaaatgccaaactaaagaagatattggccaaaataaacgctataccgagagagaaacttgatttttcaacttcctaaaacagtgttgttcaaacatttctacttatttgtacttaccagttgaacctacgtttccctaataaaatgtctatggtaaaaagttaaaaaatcctcctacttttgttagatatatttttttgtgtaattttgtaatcgttatgcggcagtaataatatacatattaatacgagttaggaatcctgtagttctcatatgctacgaggaggtattaaaaggtgcgtttcgacaatgcatctattgtagtatattattgcttaatccaaatgaatattataaatttaggaattcttgctcacattgatgcaggaaaaacttccgtaaccgagaatctgctgtttgccagtggagcaacggaaaagtgcggctgtgtggataatggtgacaccataacggactctatggatatagagaaacgtagaggaattactgttcgggcttctacgacatctattatctggaatggtgtgaaatgcaatatcattgacactccgggacacatggattttattgcggaagtggagcggacattcaaaatgcttgatggagcagtcctcatcttatccgcaaaggaaggcatacaagcgcagacaaagttgctgttcaatactttacagaagctgcaaatcccgacaattatatttatcaataagattgaccgagccggtgtgaatttggagcgtttgtatctggatataaaagcaaatctgtctcaagatgtcctgtttatgcaaaatgttgtcgatggatcggtttatccggtttgctcccaaacatatataaaggaagaatacaaagaatttgtatgcaaccatgacgacaatatattagaacgatatttggcggatagcgaaatttcaccggctgattattggaatacgataatcgctcttgtggcaaaagccaaagtctatccggtgctacatggatcagcaatgttcaatatcggtatcaatgagttgttggacgccatcacttcttttatacttcctccggcatcggtctcaaacagactttcatcttatctttataagatagagcatgaccccaaaggacataaaagaagttttctaaaaataattgacggaagtctgagacttcgagacgttgtaagaatcaacgattcggaaaaattcatcaagattaaaaatctaaaaactatcaatcagggcagagagataaatgttgatgaagtgggcgccaatgatatcgcgattgtagaggatatggatgattttcgaatcggaaattatttaggtgctgaaccttgtttgattcaaggattatcgcatcagcatcccgctctcaaatcctccgtccggccagacaggcccgaagagagaagcaaggtgatatccgctctgaatacattgtggattgaagacccgtctttgtccttttccataaactcatatagtgatgaattggaaatctcgttatatggtttaacccaaaaggaaatcatacagacattgctggaagaacgattttccgtaaaggtccattttgatgagatcaagactatatacaaagaacgacctgtaaaaaaggtcaataagattattcagatcgaagtgccgcccaacccttattgggccacaatagggctgactcttgaacccttaccgttagggacagggttgcaaatcgaaagtgacatctcctatggttatctgaaccattcttttcaaaatgccgtttttgaagggattcgtatgtcttgccaatccgggttacatggatgggaagtgactgatctgaaagtaacttttactcaagccgagtattatagcccggtaagtacacctgctgatttcagacagctgaccccttatgtcttcaggctggccttgcaacagtcaggtgtggacattctcgaaccgatgctctattttgagttgcagataccccaagcggcaagttccaaagctattacagatttgcaaaaaatgatgtctgagattgaagacatcagttgcaataatgagtggtgtcatattaaagggaaagttccattaaatacaagtaaagactatgcatcagaagtaagttcatacactaagggcttaggcatttttatggttaagccatgcgggtatcaaataacaaaaggcggttattctgataatatccgcatgaacgaaaaagataaacttttattcatgttccaaaaatcaatgtcatcaaaataatggagcggtcaggaaatttctataaggcaatacagttgggatatatacttatctccattcttatcggatgtatggcatataatagcctctatgaatggcaggagatagaagcattagaacttggcaataaaaaaatagacgcgatagtctatgatgtacacaattagggtttccgacggggtggataaggtgattgccaaatggaagaaatcaaaccccaatttgttcaagaagtataagaaaatctacaaagaattgttagagcaccctaagactgggcttgggcatcccgaagcgttaaggggcggtggggatattacatggtctcgacacattaccgcccatgaccggataatctatgacatttacgaggaagtggtagaggtctacatcttggaggtagaggggcattacaacgataaatgataaaactgttatggtcgaatattgtgtttactggttagagaacggcgagcctatgcacgaggtgttctcttctcttgccgccgccgagatgtactcatgtgcgataagagggaaagaaaacattgaatgggtggaggtgtccgaagaagaaaccattgatttggacgaactggaagacatgttcccggatgacttctgcggcgtgtaatccccttgggtagaaatggggtagaatttgttaggctgtgggggattttctaacacgccggggctttttgctgggcggttggcgtggatgtattcccatgcggcatgtgtatatatagcaagaagtgtccttgtcggacaattcttgcttttctcgctttgctcaaaaagattttaagattacctttgtggcatggaactaagacggaacgaaaagattacattccggtgtaccgaacttgaaaaggacgcacttgcggagcaggcagcccggtgcagtctaagcgtatcggaatactgccggagtttgtcccttggggggcgcccaagggagaggtacaccgaggaggaacggcagcttctccgggacatagcccagttgaaaggaacgctccaacggctgaacaactacttcggtggtcgccagtaccgggaagtgttcgaggagaaccgggcactaattacagagctcaaaaaaatactctcacgatgatagggaaagggaaaagcatatcacatggtgtggcggcattggagtacgacctcgccaaagagataaacgggcaggcagtagccaccgagatagcccggcatgaactttatgggtgtactggtgcggaaatggtgcaggaaatgaaaccgtatcacattgattttccgaacgtaaaaaacaactgcctacgtttcgaggtcagcccctcgatagaggaaagcgccacctttacggatgcggactgggcggaacttggtaacgacttcatgcagcgcatggggttggcgaaccaccagtacatcatcatccggcacagcggtacggagagcaagaaagagcaagcccacctgcacatactggcaaaccgggtttccctttccggggaactgtaccgggacaactggatagggaaaaaggcaacggaagccgccaatgccatagccaaggaacggaactttgtacagtcgcaggacattggcaaagtcaacaaagcggaaatcaaggaagccatggacggcgtattgaagaagatgcagggctttgactttaccaaattcaaggaagaacttggtaagagaggttttaaagtccgggaagccagggcaagcaccgggaaacttaacggctactatgtcacagcccgaagcggtacggagtataaggcaagcgagatagggaaaggctacactctcgcccatatcgagcggacacaaagcaaactgaagtgcaactcaatgaacatatctcatggaaacaaactcacacccggaagcggcagcttccaacgttaagcagggacagtacagcccccggaaaagaaaagatacaacgccaagggtaaacccgcagcttgcggtagaactggtgtatcaagaactaaagcgtgttgaagtctatacgaagcgcatagaggacgcaacagcccgaaaggtacaaatagacgggaaaagccttgaaagtgccgaaaatcgcctaaaaaacgtgttggcggactttgaacggcaggggtacagaatgaaaaacggcggttatgtggacaaacgaatttcgttctactcaatcctttgtgctgttatctccctattgttcgcttgtttcatgtgctatctttggacggatgcagccaaagaccgggacaactacaagcagtattatgaatactaccaagagcaggcaagggaacaaaaggggaataaataaacatctaactttacgaattatgggaacaacggaacacgaagaaccccggttcttcttcatactgaacaagggtgcgaagtccggcggcgaaatcacccatgccgtcctaaacggtagtatcgtttcgaagccggcaggctgggacgcctttcatgggcttgcactggcgagagagaaactaagcagcgaggaaatacaacagcagatgaaagaacttggtgtagaaatggaaatcgttcctctgatttgacaagattacccggagtgtttcagccgggtaatcttgttacttatccttgattttccctttaagggcgtttataatccaccctttcggattgttcttttctcgtgattttccgtttaggagagccagttctccgataaggtcggttatcttttcttgtgccgttatgaatgtctctttgttccggtttatctcttccgatgtgaagccgcaggaataacggaggtactcgtacacatggctgtctatctgatatcgtgctgtaacctttgcttgcaattctttcccttccagttcttcatctctgaactgtggttgatagaccgggtagaacctaaacccggtcaccttgctacgtttgttgtttgggttttcacgcactttcacgtagttgaaagtgtatgggcagctctcatcaagcgcagcctttgctggtttcaaaactctttcctcaaaatgatctattctgtcttttccgttcttgtccttgtatttgtccgcaggtatgccaagacggtctttcaagttttcaagggatatatccaaagggtacacctgcccactcattaacatgtaaaacctaagggaatacccagtaggtagcgcaagagccttgtttaactcaaattctctgtaccccttggcgaacttggtaaacacatcccaaaggtcgttagacacacgaaaggttataatgccagtgcgttttttatattttgggttagatataaatccgcacttccaccattcctcgtcatcttcgtaagtaaaaaaacgtcctgccaaagaatcgagtgcggctatgatttcattgtaatcacgtccggaaaaaataacgtcagatacatgcatttgtgcatcaacatcccaaagtccatgctcaaacttgcgtttcgtgcctgcataatctttcagttttactccctttagttcagcttggcacgcttccagtatgcgaagaactattctttgttcgtgtatggacatgtcttgctgttttgaccatgtatatacccatgaaacaacaacatctttattcttggtaataggtagtttctttttcataaacgattgatttgctgcaaatatacacaatccccacattaaaacaaaaaaagaggggtaaaactcccaaagtgtggggactaatttacttttaaactcccaaagtgtggggactaaactcccaaagtgtggggaaacacatgttctaatccctttatttatgggcgtttgcgaagaagtgatagagcatattacagacgatgcttataacagacaaggggaatttatagagctaagtggttgatttactgtcggaaaaagaaaaacgatagggaaaaagagcaaagcaaaaccaaccaacgggcggtaggattggaaacgggtttttatggcgtgtacacgtttgtgtacaaaatgtttgtattttattgaaatatagctatctttgtgctgaatttaaaagcaaacagatatggaagcattatcagtaagagaataccgtaacaacctcgcagcgtctttcaccaaagctgacaatggcgagcaggtactaattcgcaggaaaaacgagatttacgctttggtaaaagttggtcgtgaagatttgatgataaccccggagctgcaagcaaggattgacaaggcaagggaagaaatcaaatccggaaagtgtgttaccctcaaaagcagtgaggatattgacgcttatttcgataagcttgcatgctcccggccgccatggcggccgcgggaattcgatataggcctatgagattctctgccattattatcgctttgattgtgatgctgcctgctgtgcttagcgggcagcattattattccatggcgggagagcgaccggagacggacagcattcgtccgaacgaactctcggcatcgatccgaggtacgcttttctttcggaacaatgaatacaatgcacgttcggtcaaaggttatacgttgccgggtgcacgggtttccgcttttgcctcttactcgctgccggcagcacatggtgtgaagctttcgctcggagtatctaccctgaactactggggggcaagtcgctatccggccggtatcgcttattccgatttaccttattggacggactataacgactatgtacgcttgcgtatcctgccttatgtacaggccatgctgaagccgacggccacgactgctctcatgctgggcaatatagccggtggtacggctcacggactgatcgaaccgatctacaatcctgagttggatttgacggctgatcctgaagccggtgtgcaatttcggggtgattggacacgtttccgaatggatgtttgggtcaattggatgagcatgattttcaaaaatgacaatcatcaggagtcgtttgtctttggcttgtccactacttccaaattgttatcgggtgaaggcaaatggcgactcgaactgcccttgcaggctattgccacgcatcgcggcggggaatacaactgggcgcagcaggataccgtgcatacatgggtcaatggagctgtcggacttaagctttcgtattgccctcgtaccgacaaacccatgcagatttggggatctgcttatggtgtggcagccttgtcaagcggaggatactttccttacgaaagagggtggggcggttatctttctctcggaatggacttggagcacttcgcttttcgtaccgactattggtacggcaggcattacgtttctccctttgctgcacctttcgccaattccctgacgtatgacaaacagcctcttacgaacggttggggcgattatattcgtctctatgccgactattcgtggcggatggcacgaagtgtttcgttggcggctgttgctcgggtatggttccagccttcggatcgttttgcgatgagccacgccttggaactgacgatgcgtatcgatcccaatttcccgatagcttttctgaaaggcaatcattgatcttttcgatatttgcgagtggattcctctagaggatcccccatcgatggggtaccaggcctgtaacttcttacaggtgaatacttcttgagttcaacttataaatgcaactttttgggtgcggataataagcaataaaaacatttatttttcagagaggaaagagagacaatgtccccctttctctcactctgaatggataaagtttgctatctttgctttaattgtccgtccaaagaaaaaaaagttgcagatgagcaaacatataaccgaggaacaaaggtatgcaatttctatgatgttgcaaataccgatgagcaaaaaagcaatagcggaagctatcggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaat

SEQ ID NO: 6 - DNA, 11153 bp, Artificial Sequence
  Synthetic
tctaaatttaaatataaacaacgaattatctccttaacgtacgttttcgttccattggccctcaaaccccgttatatacattcatgtccatttatgtaaaaaatcctgctgaccttgtttatgtcttgtcagtcaccatttgcaaaaccatatttgaccctcaaagaggctgaatttgataagcaacttgctacatactcataataaggagctaaatagaacacgaatgggaaatactcaaatgccaaactaaagaagatattggccaaaataaacgctataccgagagagaaacttgatttttcaacttcctaaaacagtgttgttcaaacatttctacttatttgtacttaccagttgaacctacgtttccctaataaaatgtctatggtaaaaagttaaaaaatcctcctacttttgttagatatatttttttgtgtaattttgtaatcgttatgcggcagtaataatatacatattaatacgagttaggaatcctgtagttctcatatgctacgaggaggtattaaaaggtgcgtttcgacaatgcatctattgtagtatattattgcttaatccaaatgaatattataaatttaggaattcttgctcacattgatgcaggaaaaacttccgtaaccgagaatctgctgtttgccagtggagcaacggaaaagtgcggctgtgtggataatggtgacaccataacggactctatggatatagagaaacgtagaggaattactgttcgggcttctacgacatctattatctggaatggtgtgaaatgcaatatcattgacactccgggacacatggattttattgcggaagtggagcggacattcaaaatgcttgatggagcagtcctcatcttatccgcaaaggaaggcatacaagcgcagacaaagttgctgttcaatactttacagaagctgcaaatcccgacaattatatttatcaataagattgaccgagccggtgtgaatttggagcgtttgtatctggatataaaagcaaatctgtctcaagatgtcctgtttatgcaaaatgttgtcgatggatcggtttatccggtttgctcccaaacatatataaaggaagaatacaaagaatttgtatgcaaccatgacgacaatatattagaacgatatttggcggatagcgaaatttcaccggctgattattggaatacgataatcgctcttgtggcaaaagccaaagtctatccggtgctacatggatcagcaatgttcaatatcggtatcaatgagttgttggacgccatcacttcttttatacttcctccggcatcggtctcaaacagactttcatcttatctttataagatagagcatgaccccaaaggacataaaagaagttttctaaaaataattgacggaagtctgagacttcgagacgttgtaagaatcaacgattcggaaaaattcatcaagattaaaaatctaaaaactatcaatcagggcagagagataaatgttgatgaagtgggcgccaatgatatcgcgattgtagaggatatggatgattttcgaatcggaaattatttaggtgctgaaccttgtttgattcaaggattatcgcatcagcatcccgctctcaaatcctccgtccggccagacaggcccgaagagagaagcaaggtgatatccgctctgaatacattgtggattgaagacccgtctttgtccttttccataaactcatatagtgatgaattggaaatctcgttatatggtttaacccaaaaggaaatcatacagacattgctggaagaacgattttccgtaaaggtccattttgatgagatcaagactatatacaaagaacgacctgtaaaaaaggtcaataagattattcagatcgaagtgccgcccaacccttattgggccacaatagggctgactcttgaacccttaccgttagggacagggttgcaaatcgaaagtgacatctcctatggttatctgaaccattcttttcaaaatgccgtttttgaagggattcgtatgtcttgccaatccgggttacatggatgggaagtgactgatctgaaagtaacttttactcaagccgagtattatagcccggtaagtacacctgctgatttcagacagctgaccccttatgtcttcaggctggccttgcaacagtcaggtgtggacattctcgaaccgatgctctattttgagttgcagataccccaagcggcaagttccaaagctattacagatttgcaaaaaatgatgtctgagattgaagacatcagttgcaataatgagtggtgtcatattaaagggaaagttccattaaatacaagtaaagactatgcatcagaagtaagttcatacactaagggcttaggcatttttatggttaagccatgcgggtatcaaataacaaaaggcggttattctgataatatccgcatgaacgaaaaagataaacttttattcatgttccaaaaatcaatgtcatcaaaataatggagcggtcaggaaatttctataaggcaatacagttgggatatatacttatctccattcttatcggatgtatggcatataatagcctctatgaatggcaggagatagaagcattagaacttggcaataaaaaaatagacgcgatagtctatgatgtacacaattagggtttccgacggggtggataaggtgattgccaaatggaagaaatcaaaccccaatttgttcaagaagtataagaaaatctacaaagaattgttagagcaccctaagactgggcttgggcatcccgaagcgttaaggggcggtggggatattacatggtctcgacacattaccgcccatgaccggataatctatgacatttacgaggaagtggtagaggtctacatcttggaggtagaggggcattacaacgataaatgataaaactgttatggtcgaatattgtgtttactggttagagaacggcgagcctatgcacgaggtgttctcttctcttgccgccgccgagatgtactcatgtgcgataagagggaaagaaaacattgaatgggtggaggtgtccgaagaagaaaccattgatttggacgaactggaagacatgttcccggatgacttctgcggcgtgtaatccccttgggtagaaatggggtagaatttgttaggctgtgggggattttctaacacgccggggctttttgctgggcggttggcgtggatgtattcccatgcggcatgtgtatatatagcaagaagtgtccttgtcggacaattcttgcttttctcgctttgctcaaaaagattttaagattacctttgtggcatggaactaagacggaacgaaaagattacattccggtgtaccgaacttgaaaaggacgcacttgcggagcaggcagcccggtgcagtctaagcgtatcggaatactgccggagtttgtcccttggggggcgcccaagggagaggtacaccgaggaggaacggcagcttctccgggacatagcccagttgaaaggaacgctccaacggctgaacaactacttcggtggtcgccagtaccgggaagtgttcgaggagaaccgggcactaattacagagctcaaaaaaatactctcacgatgatagggaaagggaaaagcatatcacatggtgtggcggcattggagtacgacctcgccaaagagataaacgggcaggcagtagccaccgagatagcccggcatgaactttatgggtgtactggtgcggaaatggtgcaggaaatgaaaccgtatcacattgattttccgaacgtaaaaaacaactgcctacgtttcgaggtcagcccctcgatagaggaaagcgccacctttacggatgcggactgggcggaacttggtaacgacttcatgcagcgcatggggttggcgaaccaccagtacatcatcatccggcacagcggtacggagagcaagaaagagcaagcccacctgcacatactggcaaaccgggtttccctttccggggaactgtaccgggacaactggatagggaaaaaggcaacggaagccgccaatgccatagccaaggaacggaactttgtacagtcgcaggacattggcaaagtcaacaaagcggaaatcaaggaagccatggacggcgtattgaagaagatgcagggctttgactttaccaaattcaaggaagaacttggtaagagaggttttaaagtccgggaagccagggcaagcaccgggaaacttaacggctactatgtcacagcccgaagcggtacggagtataaggcaagcgagatagggaaaggctacactctcgcccatatcgagcggacacaaagcaaactgaagtgcaactcaatgaacatatctcatggaaacaaactcacacccggaagcggcagcttccaacgttaagcagggacagtacagcccccggaaaagaaaagatacaacgccaagggtaaacccgcagcttgcggtagaactggtgtatcaagaactaaagcgtgttgaagtctatacgaagcgcatagaggacgcaacagcccgaaaggtacaaatagacgggaaaagccttgaaagtgccgaaaatcgcctaaaaaacgtgttggcggactttgaacggcaggggtacagaatgaaaaacggcggttatgtggacaaacgaatttcgttctactcaatcctttgtgctgttatctccctattgttcgcttgtttcatgtgctatctttggacggatgcagccaaagaccgggacaactacaagcagtattatgaatactaccaagagcaggcaagggaacaaaaggggaataaataaacatctaactttacgaattatgggaacaacggaacacgaagaaccccggttcttcttcatactgaacaagggtgcgaagtccggcggcgaaatcacccatgccgtcctaaacggtagtatcgtttcgaagccggcaggctgggacgcctttcatgggcttgcactggcgagagagaaactaagcagcgaggaaatacaacagcagatgaaagaacttggtgtagaaatggaaatcgttcctctgatttgacaagattacccggagtgtttcagccgggtaatcttgttacttatccttgattttccctttaagggcgtttataatccaccctttcggattgttcttttctcgtgattttccgtttaggagagccagttctccgataaggtcggttatcttttcttgtgccgttatgaatgtctctttgttccggtttatctcttccgatgtgaagccgcaggaataacggaggtactcgtacacatggctgtctatctgatatcgtgctgtaacctttgcttgcaattctttcccttccagttcttcatctctgaactgtggttgatagaccgggtagaacctaaacccggtcaccttgctacgtttgttgtttgggttttcacgcactttcacgtagttgaaagtgtatgggcagctctcatcaagcgcagcctttgctggtttcaaaactctttcctcaaaatgatctattctgtcttttccgttcttgtccttgtatttgtccgcaggtatgccaagacggtctttcaagttttcaagggatatatccaaagggtacacctgcccactcattaacatgtaaaacctaagggaatacccagtaggtagcgcaagagccttgtttaactcaaattctctgtaccccttggcgaacttggtaaacacatcccaaaggtcgttagacacacgaaaggttataatgccagtgcgttttttatattttgggttagatataaatccgcacttccaccattcctcgtcatcttcgtaagtaaaaaaacgtcctgccaaagaatcgagtgcggctatgatttcattgtaatcacgtccggaaaaaataacgtcagatacatgcatttgtgcatcaacatcccaaagtccatgctcaaacttgcgtttcgtgcctgcataatctttcagttttactccctttagttcagcttggcacgcttccagtatgcgaagaactattctttgttcgtgtatggacatgtcttgctgttttgaccatgtatatacccatgaaacaacaacatctttattcttggtaataggtagtttctttttcataaacgattgatttgctgcaaatatacacaatccccacattaaaacaaaaaaagaggggtaaaactcccaaagtgtggggactaatttacttttaaactcccaaagtgtggggactaaactcccaaagtgtggggaaacacatgttctaatccctttatttatgggcgtttgcgaagaagtgatagagcatattacagacgatgcttataacagacaaggggaatttatagagctaagtggttgatttactgtcggaaaaagaaaaacgatagggaaaaagagcaaagcaaaaccaaccaacgggcggtaggattggaaacgggtttttatggcgtgtacacgtttgtgtacaaaatgtttgtattttattgaaatatagctatctttgtgctgaatttaaaagcaaacagatatggaagcattatcagtaagagaataccgtaacaacctcgcagcgtctttcaccaaagctgacaatggcgagcaggtactaattcgcaggaaaaacgagatttacgctttggtaaaagttggtcgtgaagatttgatgataaccccggagctgcaagcaaggattgacaaggcaagggaagaaatcaaatccggaaagtgtgttaccctcaaaagcagtgaggatattgacgcttatttcgataagcttgcatgctcccggccgccatggcggccgcgggaattcgatataggcctatgagattctctgccattattatcgctttgattgtgatgctgcctgctgtgcttagcgggcagcattattattccatggcgggagagcgaccggagacggacagcattcgtccgaacgaactctcggcatcgatccgaagtacgcttttctttcggaacaatgaatacaatgcacgttcggtcaaaggttatacgttgccgggtgcacgggtttccgcttttgcctcttactcgctgccggcagcacatggtgtgaagctttcgctcggagtatctaccctgaactactggggggcaagtcgctatccggccggtatcgcttattccgatttaccttattggacggactataacgactatgtacgcttgcgtatcctgccttatgtacaggccatgctgaagccgacggccacgactgctctcatgctgggcaatatagccggtggtacggctcacggactgatcgaaccgatctacaatcctgagttggatttgacggctgatcctgaagccggtgtgcaatttcggggtgattggacacgtttccgaatggatgtttgggtcaattggatgagcatgattttcaaaaatgacaatcatcaggagtcgtttgtctttggcttgtccactacttccaaattgttatcgggtgaaggcaaatggcgactcgaactgcccttgcaggctattgccacgcatcgcggcggggaatacaactgggcgcagcaggataccgtgcatacatgggtcaatggagctgtcggacttaagctttcgtattgccctcgtaccgacaaacccatgcagatttggggatctgcttatggtgtggcagccttgtcaagcggaggatactttccttacgaaagagggtggggcggttatctttctctcggaatggacttggagcacttcgcttttcgtaccgactattggtacggcaggcattacgtttctccctttgctgcacctttcgccaattccctgacgtatgacaaacagcctcttacgaacggttggggcgattatattcgtctctatgccgactattcgtggcggatggcacgaagtgtttcgttggcggctgttgctcgggtatggttccagccttcggatcgttttgcgatgagccacgccttggaactgacgatgcgtatcgatcccaatttcccgatagcttttctgaaaggcaatcattgatcttttcgatatttgcgagtggattcctctagaggatcccccatcgatggggtaccaggcctgtaacttcttacaggtgaatacttcttgagttcaacttataaatgcaactttttgggtgcggataataagcaataaaaacatttatttttcagagaggaaagagagacaatgtccccctttctctcactctgaatggataaagtttgctatctttgctttaattgtccgtccaaagaaaaaaaagttgcagatgagcaaacatataaccgaggaacaaaggtatgcaatttctatgatgttgcaaataccgatgagcaaaaaagcaatagcggaagctatcggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaat

SEQ ID NO: 7 - DNA, 9901 bp, Artificial Sequence
  Synthetic
tctaaatttaaatataaacaacgaattatctccttaacgtacgttttcgttccattggccctcaaaccccgttatatacattcatgtccatttatgtaaaaaatcctgctgaccttgtttatgtcttgtcagtcaccatttgcaaaaccatatttgaccctcaaagaggctgaatttgataagcaacttgctacatactcataataaggagctaaatagaacacgaatgggaaatactcaaatgccaaactaaagaagatattggccaaaataaacgctataccgagagagaaacttgatttttcaacttcctaaaacagtgttgttcaaacatttctacttatttgtacttaccagttgaacctacgtttccctaataaaatgtctatggtaaaaagttaaaaaatcctcctacttttgttagatatatttttttgtgtaattttgtaatcgttatgcggcagtaataatatacatattaatacgagttaggaatcctgtagttctcatatgctacgaggaggtattaaaaggtgcgtttcgacaatgcatctattgtagtatattattgcttaatccaaatgaatattataaatttaggaattcttgctcacattgatgcaggaaaaacttccgtaaccgagaatctgctgtttgccagtggagcaacggaaaagtgcggctgtgtggataatggtgacaccataacggactctatggatatagagaaacgtagaggaattactgttcgggcttctacgacatctattatctggaatggtgtgaaatgcaatatcattgacactccgggacacatggattttattgcggaagtggagcggacattcaaaatgcttgatggagcagtcctcatcttatccgcaaaggaaggcatacaagcgcagacaaagttgctgttcaatactttacagaagctgcaaatcccgacaattatatttatcaataagattgaccgagccggtgtgaatttggagcgtttgtatctggatataaaagcaaatctgtctcaagatgtcctgtttatgcaaaatgttgtcgatggatcggtttatccggtttgctcccaaacatatataaaggaagaatacaaagaatttgtatgcaaccatgacgacaatatattagaacgatatttggcggatagcgaaatttcaccggctgattattggaatacgataatcgctcttgtggcaaaagccaaagtctatccggtgctacatggatcagcaatgttcaatatcggtatcaatgagttgttggacgccatcacttcttttatacttcctccggcatcggtctcaaacagactttcatcttatctttataagatagagcatgaccccaaaggacataaaagaagttttctaaaaataattgacggaagtctgagacttcgagacgttgtaagaatcaacgattcggaaaaattcatcaagattaaaaatctaaaaactatcaatcagggcagagagataaatgttgatgaagtgggcgccaatgatatcgcgattgtagaggatatggatgattttcgaatcggaaattatttaggtgctgaaccttgtttgattcaaggattatcgcatcagcatcccgctctcaaatcctccgtccggccagacaggcccgaagagagaagcaaggtgatatccgctctgaatacattgtggattgaagacccgtctttgtccttttccataaactcatatagtgatgaattggaaatctcgttatatggtttaacccaaaaggaaatcatacagacattgctggaagaacgattttccgtaaaggtccattttgatgagatcaagactatatacaaagaacgacctgtaaaaaaggtcaataagattattcagatcgaagtgccgcccaacccttattgggccacaatagggctgactcttgaacccttaccgttagggacagggttgcaaatcgaaagtgacatctcctatggttatctgaaccattcttttcaaaatgccgtttttgaagggattcgtatgtcttgccaatccgggttacatggatgggaagtgactgatctgaaagtaacttttactcaagccgagtattatagcccggtaagtacacctgctgatttcagacagctgaccccttatgtcttcaggctggccttgcaacagtcaggtgtggacattctcgaaccgatgctctattttgagttgcagataccccaagcggcaagttccaaagctattacagatttgcaaaaaatgatgtctgagattgaagacatcagttgcaataatgagtggtgtcatattaaagggaaagttccattaaatacaagtaaagactatgcatcagaagtaagttcatacactaagggcttaggcatttttatggttaagccatgcgggtatcaaataacaaaaggcggttattctgataatatccgcatgaacgaaaaagataaacttttattcatgttccaaaaatcaatgtcatcaaaataatggagcggtcaggaaatttctataaggcaatacagttgggatatatacttatctccattcttatcggatgtatggcatataatagcctctatgaatggcaggagatagaagcattagaacttggcaataaaaaaatagacgcgatagtctatgatgtacacaattagggtttccgacggggtggataaggtgattgccaaatggaagaaatcaaaccccaatttgttcaagaagtataagaaaatctacaaagaattgttagagcaccctaagactgggcttgggcatcccgaagcgttaaggggcggtggggatattacatggtctcgacacattaccgcccatgaccggataatctatgacatttacgaggaagtggtagaggtctacatcttggaggtagaggggcattacaacgataaatgataaaactgttatggtcgaatattgtgtttactggttagagaacggcgagcctatgcacgaggtgttctcttctcttgccgccgccgagatgtactcatgtgcgataagagggaaagaaaacattgaatgggtggaggtgtccgaagaagaaaccattgatttggacgaactggaagacatgttcccggatgacttctgcggcgtgtaatccccttgggtagaaatggggtagaatttgttaggctgtgggggattttctaacacgccggggctttttgctgggcggttggcgtggatgtattcccatgcggcatgtgtatatatagcaagaagtgtccttgtcggacaattcttgcttttctcgctttgctcaaaaagattttaagattacctttgtggcatggaactaagacggaacgaaaagattacattccggtgtaccgaacttgaaaaggacgcacttgcggagcaggcagcccggtgcagtctaagcgtatcggaatactgccggagtttgtcccttggggggcgcccaagggagaggtacaccgaggaggaacggcagcttctccgggacatagcccagttgaaaggaacgctccaacggctgaacaactacttcggtggtcgccagtaccgggaagtgttcgaggagaaccgggcactaattacagagctcaaaaaaatactctcacgatgatagggaaagggaaaagcatatcacatggtgtggcggcattggagtacgacctcgccaaagagataaacgggcaggcagtagccaccgagatagcccggcatgaactttatgggtgtactggtgcggaaatggtgcaggaaatgaaaccgtatcacattgattttccgaacgtaaaaaacaactgcctacgtttcgaggtcagcccctcgatagaggaaagcgccacctttacggatgcggactgggcggaacttggtaacgacttcatgcagcgcatggggttggcgaaccaccagtacatcatcatccggcacagcggtacggagagcaagaaagagcaagcccacctgcacatactggcaaaccgggtttccctttccggggaactgtaccgggacaactggatagggaaaaaggcaacggaagccgccaatgccatagccaaggaacggaactttgtacagtcgcaggacattggcaaagtcaacaaagcggaaatcaaggaagccatggacggcgtattgaagaagatgcagggctttgactttaccaaattcaaggaagaacttggtaagagaggttttaaagtccgggaagccagggcaagcaccgggaaacttaacggctactatgtcacagcccgaagcggtacggagtataaggcaagcgagatagggaaaggctacactctcgcccatatcgagcggacacaaagcaaactgaagtgcaactcaatgaacatatctcatggaaacaaactcacacccggaagcggcagcttccaacgttaagcagggacagtacagcccccggaaaagaaaagatacaacgccaagggtaaacccgcagcttgcggtagaactggtgtatcaagaactaaagcgtgttgaagtctatacgaagcgcatagaggacgcaacagcccgaaaggtacaaatagacgggaaaagccttgaaagtgccgaaaatcgcctaaaaaacgtgttggcggactttgaacggcaggggtacagaatgaaaaacggcggttatgtggacaaacgaatttcgttctactcaatcctttgtgctgttatctccctattgttcgcttgtttcatgtgctatctttggacggatgcagccaaagaccgggacaactacaagcagtattatgaatactaccaagagcaggcaagggaacaaaaggggaataaataaacatctaactttacgaattatgggaacaacggaacacgaagaaccccggttcttcttcatactgaacaagggtgcgaagtccggcggcgaaatcacccatgccgtcctaaacggtagtatcgtttcgaagccggcaggctgggacgcctttcatgggcttgcactggcgagagagaaactaagcagcgaggaaatacaacagcagatgaaagaacttggtgtagaaatggaaatcgttcctctgatttgacaagattacccggagtgtttcagccgggtaatcttgttacttatccttgattttccctttaagggcgtttataatccaccctttcggattgttcttttctcgtgattttccgtttaggagagccagttctccgataaggtcggttatcttttcttgtgccgttatgaatgtctctttgttccggtttatctcttccgatgtgaagccgcaggaataacggaggtactcgtacacatggctgtctatctgatatcgtgctgtaacctttgcttgcaattctttcccttccagttcttcatctctgaactgtggttgatagaccgggtagaacctaaacccggtcaccttgctacgtttgttgtttgggttttcacgcactttcacgtagttgaaagtgtatgggcagctctcatcaagcgcagcctttgctggtttcaaaactctttcctcaaaatgatctattctgtcttttccgttcttgtccttgtatttgtccgcaggtatgccaagacggtctttcaagttttcaagggatatatccaaagggtacacctgcccactcattaacatgtaaaacctaagggaatacccagtaggtagcgcaagagccttgtttaactcaaattctctgtaccccttggcgaacttggtaaacacatcccaaaggtcgttagacacacgaaaggttataatgccagtgcgttttttatattttgggttagatataaatccgcacttccaccattcctcgtcatcttcgtaagtaaaaaaacgtcctgccaaagaatcgagtgcggctatgatttcattgtaatcacgtccggaaaaaataacgtcagatacatgcatttgtgcatcaacatcccaaagtccatgctcaaacttgcgtttcgtgcctgcataatctttcagttttactccctttagttcagcttggcacgcttccagtatgcgaagaactattctttgttcgtgtatggacatgtcttgctgttttgaccatgtatatacccatgaaacaacaacatctttattcttggtaataggtagtttctttttcataaacgattgatttgctgcaaatatacacaatccccacattaaaacaaaaaaagaggggtaaaactcccaaagtgtggggactaatttacttttaaactcccaaagtgtggggactaaactcccaaagtgtggggaaacacatgttctaatccctttatttatgggcgtttgcgaagaagtgatagagcatattacagacgatgcttataacagacaaggggaatttatagagctaagtggttgatttactgtcggaaaaagaaaaacgatagggaaaaagagcaaagcaaaaccaaccaacgggcggtaggattggaaacgggtttttatggcgtgtacacgtttgtgtacaaaatgtttgtattttattgaaatatagctatctttgtgctgaatttaaaagcaaacagatatggaagcattatcagtaagagaataccgtaacaacctcgcagcgtctttcaccaaagctgacaatggcgagcaggtactaattcgcaggaaaaacgagatttacgctttggtaaaagttggtcgtgaagatttgatgataaccccggagctgcaagcaaggattgacaaggcaagggaagaaatcaaatccggaaagtgtgttaccctcaaaagcagtgaggatattgacgcttatttcgataagcttgcatgcccctcgaggggtcgactctagaggatcccccatcgatggggtaccaggcctgtaacttcttacaggtgaatacttcttgagttcaacttataaatgcaactttttgggtgcggataataagcaataaaaacatttatttttcagagaggaaagagagacaatgtccccctttctctcactctgaatggataaagtttgctatctttgctttaattgtccgtccaaagaaaaaaaagttgcagatgagcaaacatataaccgaggaacaaaggtatgcaatttctatgatgttgcaaataccgatgagcaaaaaagcaatagcggaagctatcggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaat

SEQ ID NO: 8 - DNA, 21 bp, Artificial Sequence
  synthetic
agactcaatagcagatcgacg

SEQ ID NO: 9 - DNA, 33 bp, Artificial Sequence
  synthetic
ttcataccgcccaaggatcctgttgctgtcata

SEQ ID NO: 10 - DNA, 33 bp, Artificial Sequence
  synthetic
tatgacagcaacaggatccttgggcggtatgaa

SEQ ID NO: 11 - DNA, 19 bp, Artificial Sequence
  Synthetic
gacccaaacatccattcgg

SEQ ID NO: 12 - DNA, 19 bp, Artificial Sequence
  Synthetic
tctcatacatggcacgcat

SEQ ID NO: 13 - DNA, 19 bp, Artificial Sequence
  Synthetic
atgcgtgccatgtatgaga

SEQ ID NO: 14 - DNA, 33 bp, Artificial Sequence
  Synthetic
ggactgtcaaaggaggatcctaagcacagcagg

SEQ ID NO: 15 - DNA, 33 bp, Artificial Sequence
  Synthetic
cctgctgtgcttaggatcctcctttgacagtcc

SEQ ID NO: 16 - DNA, 17 bp, Artificial Sequence
  Synthetic
ccgtaaccgggtacgat

SEQ ID NO: 17 - DNA, 19 bp, Artificial Sequence
  Synthetic
gacccaaacatccattcgg

SEQ ID NO: 18 - DNA, 27 bp, Artificial Sequence
  Synthetic
atccatggacgcccgatacccatactc

SEQ ID NO: 19 - DNA, 37 bp, Artificial Sequence
  Synthetic
cacaaagtctccgagtggatccattcagagaaccacg

SEQ ID NO: 20 - DNA, 37 bp, Artificial Sequence
  Synthetic
cgtggttctctgaatggatccactcggagactttgtg

SEQ ID NO: 21 - DNA, 27 bp, Artificial Sequence
  Synthetic
tatgtcgacaacaggttgtccgtcagc

SEQ ID NO: 22 - DNA, 20 bp, Artificial Sequence
  Synthetic
aacttcctaactgctggcac

SEQ ID NO: 23 - DNA, 20 bp, Artificial Sequence
  Synthetic
aaggcttgatgctgaagacc

SEQ ID NO: 24 - DNA, 42 bp, Artificial Sequence
  Synthetic
gtcatttcttattaagaataggatcctatttacgttgcgagc

SEQ ID NO: 25 - DNA, 42 bp, Artificial Sequence
  Synthetic
gctcgcaacgtaaataggatcctattcttaataagaaatgac

SEQ ID NO: 26 - DNA, 19 bp, Artificial Sequence
  Synthetic
tctttgccggcatctttgc

SEQ ID NO: 27 - DNA, 33 bp, Artificial Sequence
  Synthetic
ataggcctatgagattctctgccattattatcg

SEQ ID NO: 28 - DNA, 28 bp, Artificial Sequence
  Synthetic
attctagaggaatccactcgcaaatatc

SEQ ID NO: 29 - DNA, 31 bp, Artificial Sequence
  Synthetic
ggaattcggccgccgatagcttccgctattg

SEQ ID NO: 30 - DNA, 36 bp, Artificial Sequence
  Synthetic
taggtaccaggcctgtaacttcttacaggtgaatac

(19) United States
(12) Patent Application Publication
(10) Pub. No.: US 2021/0024975 A1
(43) Pub. Date: Jan. 28, 2021

(54) METHODS FOR IDENTIFYING INHIBITORS OF LIPID A DEACYLASE AND EXPRESSION VECTORS RELATED TO SAME

(71) Applicant: UNIVERSITY OF WASHINGTON, Seattle, WA (US)

(72) Inventors: Richard P. DARVEAU, Seattle, WA (US); Sumita JAIN, Seattle, WA (US)

(21) Appl. No.: 16/888,696

(22) Filed: May 30, 2020

Related U.S. Application Data
(60) Provisional application No. 62/855,839, filed on May 31, 2019.

(51) Int. Cl. C12Q 1/44; C12N 15/74; C12N 9/18; C12Q 1/02

(52) U.S. Cl. CPC C12Q 1/44; C12Q 1/025; C12N 9/18; C12N 15/74

(57) ABSTRACT

In one embodiment, an expression vector for expressing P. gingivalis lipid A deacylase a novel screen to identify P. gingivalis deacylase inhibitors. P. gingivalis deacylase inhibitors will be identified by determining the TLR4 activity of B. thetaiotaomicron strain Bt pSJ836 that expresses the P. gingivalis deacylase gene. This strain demonstrates significantly less potent TLR4 activity when compared to its isogenic strain Bt pSJ46 that does not contain the P. gingivalis deacylase but rather contains the control plasmid vector only. Consequently, the potent TLR4 activity normally found with B. thetaiotaomicron is significantly reduced only when the P. gingivalis deacylase is present. Therefore, compounds that inhibit the P. gingivalis deacylase activity can be identified in B. thetaiotaomicron strain Bt pSJ836 by an increase in TLR4 activity. Compounds that increase TLR4 activity nonspecifically in B. thetaiotaomicron strain Bt pSJ836 will be identified by comparing the TLR4 activity to B. thetaiotaomicron strain Bt pSJ46 that does not contain the P. gingivalis deacylase.

### CROSS-REFERENCE TO RELATED APPLICATIONS

This application claims the benefit of U.S. Provisional Application No. 62/855,839, filed May 31, 2019, which is incorporated herein by reference in its entirety.

### STATEMENT REGARDING FEDERALLY SPONSORED RESEARCH

This invention was made with government support under Grant Number R21DE026344, awarded by the National Institutes of Health. The government has certain rights in the invention.

### SEQUENCE LISTING

This application contains a Sequence Listing, which was submitted in ASCII format via EFS-Web, and is hereby incorporated by reference in its entirety. The ASCII copy, created on Oct. 8, 2020, is named Revised Sequence Listing 072227-8147.US01_ST25 and is 56 KB in size.

### BACKGROUND

Porphyromonas gingivalis is an anaerobic Gram-negative bacterium found in sub-gingival plaque. It is closely associated with periodontitis, a chronic inflammatory disorder responsible for major tooth loss world-wide (1). P. gingivalis is found in higher abundance in diseased periodontal pockets than in healthy pockets (2, 3) indicating it is adept at withstanding hostile immune conditions characteristic of periodontal diseased sites. The bacterium has been shown to express multiple virulence factors that enable it to evade, actively suppress and modulate the host innate immune response (4-13), giving it the ability to persist in inflammatory conditions and to become a recurrent member of the bacterial community during disease. Furthermore, P. gingivalis infection in mouse and rabbit models have shown it to alter the composition of the sub-gingival microbiome, and augment bacterial load, marking it as a keystone pathogen (14-17) with the capacity to trigger microbial dysbiosis. A consequence of dysbiosis, commonly seen in chronic inflammatory disorders, is breakdown of the homeostatic bacterial-host balance leading to aggravated immune responses that, in the case of periodontitis, manifests in loss of supporting structures of the tooth.

P. gingivalis is unusually adept at evading the innate immune response mediated by Toll-like receptor 4 (TLR4)(1, 18, 19), a major bacterial clearance mechanism mounted by the host. TLR4 is a host innate immune receptor that, together with its co-receptor myeloid differentiation factor-2 (MD-2), recognizes lipopolysaccharide (LPS), an essential macromolecule that forms the outer layer of the outer membrane in Gram-negative bacteria (20-23). The TLR4 ligand specifically is lipid A, a hydrophobic glycolipid anchor of LPS. Lipid A satisfies the requirements first outlined by Janeway et al for pattern recognition receptor ligands in that the structure is highly conserved, essential for bacterial survival, and differs significantly from host (self) structures (24). Engagement of bacterial lipid A by the TLR4/MD-2 complex initiates a pro-inflammatory response, which in turn leads to production of co-stimulatory molecules required for adaptive immunity, culminating in clearance of local infection. The prototypical lipid A structure, first investigated in E. coli, is comprised by a disaccharide, β-1′,6-glucosamine, to which is attached six C12-C14 fatty acyl chains and two phosphate groups at C1- and C4′-positions (25). E. coli lipid A is highly immunostimulatory, capable of triggering endotoxic shock even when encountered in small quantities in the bloodstream.

In striking contrast, P. gingivalis lipid A is a poor activator of TLR4 (26-28). The bacterium exhibits a heterogeneous population of lipid A structures comprising penta- and tetra-acylated molecules that are bis-, mono- or non-phosphorylated. The initial structure synthesized is penta-acylated bis-phosphorylated with C15-C17 acyl chain lengths (FIG. 1), a moderate TLR4 agonist (29). Under standard growth conditions it is subject to the action of three lipid A modifying enzymes leading to a lipid A profile that is heavily biased towards a tetra-acylated non-phosphorylated structure (29). The three distinct enzymatic activities that modify P. gingivalis lipid A are C1-phosphatase, C4′-phosphatase and 3-O-deacylase (FIG. 1). When P. gingivalis is grown under conditions of high hemin, hemin being an iron source required for growth, the C1-phosphatase is inactive leading to a shift of tetra-acylated non-phosphorylated lipid A to tetra-acylated C1-phosphorylated lipid A (30). Notably, both tetra-acylated structures are TLR4-evasive at the TLR4/MD-2 receptor complex. Non-phosphorylated tetra-acylated lipid A is inert for TLR4 activation, whereas C1-phosphorylated tetra-acylated lipid A is an antagonist of TLR4 activation (29, 31). Antagonism of TLR4 has the potential to have a community-wide effect since it can dampen the host response to any Gram-negative member of the oral community. The two tetra-acylated structures are, in short, are believed to be key to the ability of P. gingivalis to effectively evade the host TLR4 immune attack.

The C1- and C4′-phosphatases were identified by homology searches, to be encoded by PG1773 and PG1587 respectively (W83 designations) (29). Recently, PG0027 was identified to possess C1-phosphatase activity as well (32). Deletion of the C1-phosphatase, PG1773, led to accumulation of C1-phosphorylated, tetra-acylated lipid A, the TLR4 antagonist. Deletion of the C4′-phosphatase, PG1587, on the other hand, resulted in formation of C4′-phosphorylated lipid A, but was exclusively penta-acylated, suggesting the C4′-phosphate moiety needs to be removed for lipid A deacylation to occur. The contribution of lipid A deacylation to attenuation of TLR4-dependent pro-inflammatory responses, and for facilitating P. gingivalis persistence, has been experimentally demonstrated in vivo in a murine model of infection comparing wild-type vs. ΔPG1587 mutants (33).

The gene encoding lipid A deacylase, has been found to be important to P. gingivalis' ability to evade TLR4 sensing but has previously remained unidentified. Thus, it is desired to identify the P. gingivalis lipid A deacylase gene and to develop inhibitors against lipid A deacylase.

### SUMMARY

In one embodiment, a plasmid expression vector is provided. The plasmid expression vector may include a portion of a tetC gene; a Bacteroides promoter (e.g., an ermF gene promoter, which may be a nucleotide sequence that includes at least 300 base pairs upstream of the ermF gene; a multiple cloning site (MCS) downstream of the Bacteroides promoter wherein the MCS include one or more restriction sites including, but not limited to, StuI, XbaI, and SphI; and a bacterial gene (e.g., a P. gingivalis lipid A deacylase gene) inserted or cloned into the MCS; wherein the plasmid expression vector is capable of expressing the bacterial gene in a host bacterial cell belonging to the Order Bacteroidales (e.g., a B. thetaiotaomicron cell).

The plasmid expression vector of claim 1, further comprising one or more genes selected from a bla gene; a rep sequence (origin of replication, not a gene); a repA sequence; an mbpA gene; an mbpB gene; an mbpC gene; a metR gene; and a tetQ gene.

In another embodiment, a bacterial cell that includes the plasmid expression vector (i.e., transformed cell) described above is provided. In another embodiment, a bacterial cell culture system for screening inhibitors of lipid A deacylase is provided. That system may include a population of bacterial cells that includes an expression vector (i.e., transformed cells) as described herein. In one embodiment, the bacterial cell that includes or expresses the bacteria gene is from a different Genus and/or Species than the host cell. For example, in one embodiment, the bacterial gene is a P. gingivalis lipid A deacylase gene and the host cell is a B. thetaiotaomicron cell.

A bacterial cell that expresses P. gingivalis lipid A deacylase gene may also be used in a method of screening inhibitors of lipid A deacylase. Such methods may include steps of contacting a population of bacterial cells comprising an expression vector with a candidate compound; measuring TLR4 activity in the population of bacterial cells; and identifying the candidate compound as a deacylase inhibitor when TLR4 activity in the population of bacterial cells after contact with the candidate compound is increased as compared to TLR4 activity in the population of bacterial cells before contact with the candidate compound.

### BRIEF DESCRIPTION OF THE DRAWINGS

FIG. 1 shows modification of P. gingivalis lipid A structure according to some embodiments. Penta-acylated bis-phosphorylated lipid A (left), a moderate TLR4 agonist, is the initial lipid A structure synthesized by P. gingivalis. It is modified to tetra-acylated non-phosphorylated lipid A (right), silent for TLR4 activation, by the action of C1- and C4′-phosphatases and a 3-O-deacylase. Inhibition of C1-phosphatase activity leads to the formation of C1-phosphorylated tetra-acylated lipid A, a TLR4 antagonist.

FIGS. 2A-2B show that a J5-c5 transposon mutant induces a pro-inflammatory response according to some embodiments. IL-6 secreted by MM6 cells in response to exposure to 106 or 107 intact P. gingivalis 33277 bacteria versus (FIG. 2A) isogenic ΔPG1587, ΔPG1773 or Δkgp mutants, and (FIG. 2B) J5-c5 transposon mutant. The y-axis shows amount of IL-6 secreted in pg/ml. The results are means±standard deviations (SD) of triplicate samples from one of two independent experiments. Asterisks denote significant differences in amount of IL-6 secreted relative to the wild-type 33277 control (P<0.01; 2-tailed unpaired t-tests).

FIGS. 3A-3C show that J5-c5 mutants stimulate TLR4 more than TLR2. In FIGS. 3A-3B, HEK293 cells expressing TLR4/MD-2 (FIG. 3A) or TLR2/mCD14 (FIG. 3B) were infected with wild-type 33277, ΔPG1587 or J5-c5 intact bacterial cells; HEK293 cells expressing TLR4/MD-2 were exposed to LPS isolated from wild-type, ΔPG1587 or J5-c5 transposon mutant (FIG. 3C). Fold NF-κB stimulation of infected cells relative to unstimulated control is plotted on the y axis. The results are means±SD for triplicate samples from one of two independent experiments. Asterisks denote a significant increase in fold NF-κB stimulation relative to wild-type 33277 control (P<0.05; 2-tailed unpaired t-tests).

FIGS. 4A-4D shows that lipid A of the J5-c5 mutant is predominantly penta-acylated. MALDI-TOF mass spectrometry of lipid A structure was conducted in positive (FIGS. 4A-4B) and negative ion modes (FIGS. 4C-4D), which detect non-phosphorylated and phosphorylated structures respectively, according to some embodiments. Tetraacylated non-phosphorylated lipid A is seen in wild-type 33277 (FIG. 4A) but not in J5-c5 mutant (FIG. 4B), which instead exhibits penta-acylated non-phosphorylated lipid A. Under growth in high-hemin conditions, tetra-acylated mono-phosphorylated antagonist is detected in wild-type (FIG. 4C) but not in J5-c5 mutant (FIG. 4D), which instead displays penta-acylated lipid A that is either mono- or bis-phosphorylated (m/z 1769). Tetra-acylated lipid A is not detected in either condition in the J5-c5 mutant. Numbers refer to the m/z ratio of the predominant peak in each lipid A cluster.

FIG. 5 is a schematic diagram of a pT-COW vector according to some embodiments.

FIG. 6 is a schematic diagram of a pSJ46 plasmid.

FIG. 7 is a schematic diagram of a pSJ836 plasmid according to some embodiments. This schematic also corresponds to a pSJ922 plasmid according to some embodiments.

FIGS. 8A-8B show that the ΔPGN_1123 mutant is devoid of tetra-acylated lipid A according to some embodiments. In FIG. 8A, Lipid A preparations of ΔPGN_1123::ermF mutants exhibit penta-acylated non-phosphorylated lipid A as detected by MALDI-TOF MS in positive ion mode, distinctly devoid of tetra-acylated lipid A. In FIG. B, ΔPGN_1123 mutants expressing the PGN_1123 gene in trans on pSJ836 display tetraacylated non-phosphorylated lipid A, similar to wild-type, indicating complementation of lipid A deacylase function. Numbers refer to the m/z ratio of the predominant peak in each lipid A cluster.

FIG. 9 shows that ΔPGN_1123::ermF mutants stimulate TLR4 according to some embodiments. HEK293 cells expressing TLR4/MD-2 were stimulated with wild-type 33277, J5-c5 transposon mutant, isogenic ΔPGN_1124, ΔPGN_1123 or ΔPGN_1123 (pSJ836) whole bacterial cells. The last sample, ΔPGN_1123 (pSJ836), expresses PGN_1123 from pSJ836 in trans. Fold NF-κB stimulation of infected cells relative to unstimulated control is plotted on the y axis. The results are means+SD of triplicate samples from one of three independent experiments. Asterisks denote a significant increase in fold NF-κB stimulation relative to wild-type 33277 control (P<0.05; 2-tailed unpaired t-tests).

FIG. 10 shows that PGN_1123 is in an operon with the upstream PGN_1125 and PGN_1124 genes according to some embodiments. RT-PCR of mRNA from wild-type 33277 using primers a-b, overlapping PGN_1125-1124, lane 2; primers c-d, overlapping PGN_1124-1123, lane 4; primers a-d, overlapping PGN_1125-1123, lane 6. Reverse transcriptase is absent in lanes 3, 5, 7, which use the same primer sets as lanes 2, 4 and 6 respectively. Molecular weight ladder is in lane 1.

FIGS. 11A-11C show that B. thetaiotaomicron lipid A is deacylated by PGN_1123 according to some embodiments. Lipid A structure of B. thetaiotaomicron with empty plasmid vector, pSJ46 (FIG. 11A), or PGN_1123-expressing plasmid pSJ836 as detected by MALDI-TOF MS in negative ion mode (FIG. 11B). IN FIG. 11C, HEK293 cells expressing TLR4/MD-2 were stimulated with B. thetaiotaomicron (pSJ46) or B. thetaiotaomicron (pSJ836) intact bacteria. Fold NF-κB stimulation of infected cells relative to unstimulated control is plotted on the y axis. The results are means+SD of triplicate samples from one of two independent experiments. Asterisks denote a significant decrease in fold NF-κB stimulation relative to Bt (pSJ46) (P<0.01; 2-tailed unpaired t-tests).

FIG. 12 is the nucleotide sequence for a pSJ46 plasmid according to one embodiment (SEQ ID NO:7). Sequences for individual genes or other elements (e.g., promoters) that are part of the plasmid are underlined as indicated. For reference, a “|” symbol is placed every 10 nucleotides.

FIG. 13 is the nucleotide sequence for a pSJ836 plasmid according to one embodiment (SEQ ID NO:5). Sequences for individual genes or other elements (e.g., promoters) that are part of the plasmid are underlined as indicated. The start codon for PGN_1123 is underlined in bold at position 6854-6857. For reference, a “|” symbol is placed every 10 nucleotides.

FIG. 14 is the nucleotide sequence for a pSJ922 plasmid according to one embodiment (SEQ ID NO:6). Sequences for individual genes or other elements (e.g., promoters) that are part of the plasmid are underlined as indicated. The start codon for PGN_1123 is underlined in bold at position 6854-6857. For reference, a “|” symbol is placed every 10 nucleotides.

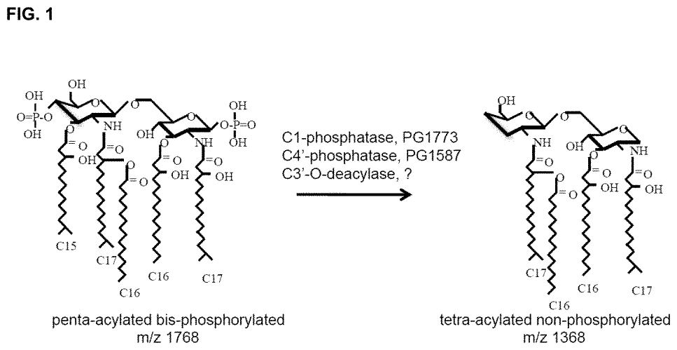

### DETAILED DESCRIPTION

Periodontitis, commonly referred to as gum disease, is a chronic inflammatory condition that affects a large proportion of the population. Porphyromonas gingivalis is a bacterium closely associated with periodontitis although how and if it is a cause for the disease is not known. It has a formidable capacity to dampen the host's innate immune response enabling its persistence in diseased sites, and triggering microbial dysbiosis in animal models of infection. P. gingivalis is particularly adept at evading the host's TLR4-mediated innate immune response by modifying the structure of lipid A, the TLR4 ligand. In the embodiments discussed herein, the gene encoding lipid A deacylase is identified, a key enzyme that modifies lipid A to TLR4-evasive structures. The present disclosure relates to an expression vector for expressing a bacterial gene in a bacterial cell, cell culture systems relating to bacterial cells that express exogenous genes, and methods for screening candidate compounds using the same.

In one embodiment, the expression vector disclosed herein is a plasmid expression vector. The plasmid vector includes a bacterial gene that can be expressed in a host cell. While the host cell and the bacterial gene may be from the same Genus, or Species of bacteria, in certain embodiments, the host cell is a first Genus or first Species and the bacterial gene is from a second Genus or a second Species. For example, in some embodiments, the bacterial gene is from a bacterium that expresses lipid A deacylase and the host cell is from a bacterium that does not express lipid A deacylase. In some embodiments, the bacterial gene is from a bacterium belonging to the Genus Porphyromonas and the host cell is a bacterial cell belonging to the Genus Bacteriodes (e.g., a B. thetaiotaomicron cell).

In one embodiment, the bacterial gene that is included as part of the expression vector is a P. gingivalis lipid A deacylase gene. In another embodiment, the bacterial gene is a P. gingivalis lipid A deacylase gene at the locus tag of PGN_1123—also known as PGN_RS05380 in P. gingivalis strain 33277. In some embodiments, the P. gingivalis lipid A deacylase gene includes all or a portion of the following nucleotide sequence:

(SEQ ID NO: 1)
ATGAGATTCTCTGCCATTATTATCGCTTTGATTGTGATGCTGCCTGCT
GTGCTTAGCGGGCAGCATTATTATTCCATGGCGGGAGAGCGACCGG
AGACGGACAGCATTCGTCCGAACGAACTCTCGGCATCGATCCGAAG
TACGCTTTTCTTTCGGAACAATGAATACAATGCACGTTCGGTCAAAGG
TTATACGTTGCCGGGTGCACGGGTTTCCGCTTTTGCCTCTTACTCGC
TGCCGGCAGCACATGGTGTGAAGCTTTCGCTCGGAGTATCTACCCTG
AACTACTGGGGGGCAAGTCGCTATCCGGCCGGTATCGCTTATTCCGA
TTTACCTTATTGGACGGACTATAACGACTATGTACGCTTGCGTATCCT
GCCTTATGTACAGGCCATGCTGAAGCCGACGGCCACGACTGCTCTC
ATGCTGGGCAATATAGCCGGTGGTACGGCTCACGGACTGATCGAAC
CGATCTACAATCCTGAGTTGGATTTGACGGCTGATCCTGAAGCCGGT
GTGCAATTTCGGGGTGATTGGACACGTTTCCGAATGGATGTTTGGGT
CAATTGGATGAGCATGATTTTCAAAAATGACAATCATCAGGAGTCGTT
TGTCTTTGGCTTGTCCACTACTTCCAAATTGTTATCGGGTGAAGGCAA
ATGGCGACTCGAACTGCCCTTGCAGGCTATTGCCACGCATCGCGGC
GGGGAATACAACTGGGCGCAGCAGGATACCGTGCATACATGGGTCA
ATGGAGCTGTCGGACTTAAGCTTTCGTATTGCCCTCGTACCGACAAA
CCCATGCAGATTTGGGGATCTGCTTATGGTGTGGCAGCCTTGTCAAG
CGGAGGATACTTTCCTTACGAAAGAGGGTGGGGCGGTTATCTTTCTC
TCGGAATGGACTTGGAGCACTTCGCTTTTCGTACCGACTATTGGTAC
GGCAGGCATTACGTTTCTCCCTTTGCTGCACCTTTCGCCAATTCCCT
GACGTATGACAAACAGCCTCTTACGAACGGTTGGGGCGATTATATTC
GTCTCTATGCCGACTATTCGTGGCGGATGGCACGAAGTGTTTCGTTG
GCGGCTGTTGCTCGGGTATGGTTCCAGCCTTCGGATCGTTTTGCGAT
GAGCCACGCCTTGGAACTGACGATGCGTATCGATCCCAATTTCCCGA
TAGCTTTTCTGAAAGGCAATCATTGA

In some embodiments, the P. gingivalis lipid A deacylase gene includes all or a portion of the following nucleotide sequence:

(SEQ ID NO: 2)
ATGAGATTCTCTGCCATTATTATCGCTTTGATTGTGATGCTGCCTGCT
GTGCTTAGCGGGCAGCATTATTATTCCATGGCGGGAGAGCGACCGG
AGACGGACAGCATTCGTCCGAACGAACTCTCGGCATCGATCCGAgGT
ACGCTTTTCTTTCGGAACAATGAATACAATGCACGTTCGGTCAAAGGT
TATACGTTGCCGGGTGCACGGGTTTCCGCTTTTGCCTCTTACTCGCT
GCCGGCAGCACATGGTGTGAAGCTTTCGCTCGGAGTATCTACCCTGA
ACTACTGGGGGGCAAGTCGCTATCCGGCCGGTATCGCTTATTCCGAT
TTACCTTATTGGACGGACTATAACGACTATGTACGCTTGCGTATCCTG
CCTTATGTACAGGCCATGCTGAAGCCGACGGCCACGACTGCTCTCAT
GCTGGGCAATATAGCCGGTGGTACGGCTCACGGACTGATCGAACCG
ATCTACAATCCTGAGTTGGATTTGACGGCTGATCCTGAAGCCGGTGT
GCAATTTCGGGGTGATTGGACACGTTTCCGAATGGATGTTTGGGTCA
ATTGGATGAGCATGATTTTCAAAAATGACAATCATCAGGAGTCGTTTG
TCTTTGGCTTGTCCACTACTTCCAAATTGTTATCGGGTGAAGGCAAAT
GGCGACTCGAACTGCCCTTGCAGGCTATTGCCACGCATCGCGGCGG
GGAATACAACTGGGCGCAGCAGGATACCGTGCATACATGGGTCAAT
GGAGCTGTCGGACTTAAGCTTTCGTATTGCCCTCGTACCGACAAACC
CATGCAGATTTGGGGATCTGCTTATGGTGTGGCAGCCTTGTCAAGCG
GAGGATACTTTCCTTACGAAAGAGGGTGGGGCGGTTATCTTTCTCTC
GGAATGGACTTGGAGCACTTCGCTTTTCGTACCGACTATTGGTACGG
CAGGCATTACGTTTCTCCCTTTGCTGCACCTTTCGCCAATTCCCTGAC
GTATGACAAACAGCCTCTTACGAACGGTTGGGGCGATTATATTCGTC
TCTATGCCGACTATTCGTGGCGGATGGCACGAAGTGTTTCGTTGGCG
GCTGTTGCTCGGGTATGGTTCCAGCCTTCGGATCGTTTTGCGATGAG
CCACGCCTTGGAACTGACGATGCGTATCGATCCCAATTTCCCGATAG
CTTTTCTGAAAGGCAATCATTGA

The sequence of SEQ ID NO:2 differs from SEQ ID NO:1 by a single nucleotide change: SEQ ID NO:2 has a G in place of an A at the 139th bp (shown as a lowercase g in SEQ ID NO:2 above), which translates to a glycine at the 47th amino acid position when expressed. As such, minor alterations in the sequence of the gene are also included as part of the disclosure herein. Such minor alterations may include point mutations, deletions, insertions that do not significantly alter the protein, peptide, or enzyme expressed by the plasmid expression vector. In some embodiments, minor alterations may result in a nucleotide sequence that differs from SEQ ID NO:1 by a single nucleotide change, by two or more nucleotide changes. In other embodiments, the minor alterations may result in a sequence that has about 90% homology, about 95% homology, about 96% homology, about 97% homology, about 98% homology, or about 99% homology to SEQ ID NO:1 or SEQ NO ID:2. The minor alterations in the embodiments disclosed herein may include changes that do not alter the protein sequence based on the genetic code or alternatively, do not significantly alter the protein sequence such that the activity or utility of the protein is lost.

The P. gingivalis lipid A deacylase genes above (SEQ ID NO:1, SEQ ID NO:2) are in the reverse orientation as compared to the bacterial promoter. In other embodiments, the P. gingivalis lipid A deacylase gene is a reverse complement to SEQ ID NO:1 or SEQ ID NO:2.

In some embodiments, the expression vector expresses or is capable of expressing the P. gingivalis lipid A deacylase gene in the host cell. In such embodiments, the lipid A deacylase enzyme expressed in the host cell includes all or a portion of the following amino acid sequence (which is encoded by SEQ ID NO:1):

(SEQ ID NO: 3)
MRFSAIIIALIVMLPAVLSGQHYYSMAGERPETDSIRPNELSASIRST
LFFRNNEYNARSVKGYTLPGARVSAFASYSLPAAHGVKLSLGVSTLNY
WGASRYPAGIAYSDLPYWTDYNDYVRLRILPYVQAMLKPTATTALMLG
NIAGGTAHGLIEPIYNPELDLTADPEAGVQFRGDWTRFRMDVWVNWMS
MIFKNDNHQESFVFGLSTTSKLLSGEGKWRLELPLQAIATHRGGEYNW
AQQDTVHTWVNGAVGLKLSYCPRTDKPMQIWGSAYGVAALSSGGYFPY
ERGWGGYLSLGMDLEHFAFRTDYWYGRHYVSPFAAPFANSLTYDKQPL
TNGWGDYIRLYADYSWRMARSVSLAAVARVWFQPSDRFAMSHALELTM
RIDPNFPIAFLKGNH
(potential active sites underlined)

In other embodiments, the lipid A deacylase enzyme expressed in the host cell includes all or a portion of the following amino acid sequence (which is encoded by SEQ ID NO:2):

(SEQ ID NO: 4)
MRFSAIIIALIVMLPAVLSGQHYYSMAGERPETDSIRPNELSASIRGT
LFFRNNEYNARSVKGYTLPGARVSAFASYSLPAAHGVKLSLGVSTLNY
WGASRYPAGIAYSDLPYWTDYNDYVRLRILPYVQAMLKPTATTALMLG
NIAGGTAHGLIEPIYNPELDLTADPEAGVQFRGDWTRFRMDVWVNWMS
MIFKNDNHQESFVFGLSTTSKLLSGEGKWRLELPLQAIATHRGGEYNW
AQQDTVHTWVNGAVGLKLSYCPRTDKPMQIWGSAYGVAALSSGGYFPY
ERGWGGYLSLGMDLEHFAFRTDYWYGRHYVSPFAAPFANSLTYDKQPL
TNGWGDYIRLYADYSWRMARSVSLAAVARVWFQPSDRFAMSHALELTM
RIDPNFPIAFLKGNH
(potential active sites underlined)

The lipid A deacylase gene encodes the enzyme responsible for modifying lipid A to TLR4-evasive structures in P. gingivalis. Unlike most bacteria that exhibit a form of lipid A that activates the TLR4-mediated immune response, wild type P. gingivalis evades TLR4-mediated immune response by producing modified versions of lipid A, some of which can antagonize the TLR4 response whereas others fail to elicit TLR4 activation.

To express the desired bacterial gene, the plasmid expression vector includes one or more promoters to transcribe the gene. In some embodiments, the one or more promoter is a suitable wild type, engineered, inducible, constitutive, or repressible bacterial promoter from a bacterial gene known in the art including, but not limited to, T7, Sp6, lac, araBad, trp, and Ptac. In certain embodiments, the one or more promoters is any suitable wild type, engineered, inducible, constitutive, or repressible Bacteroides promoter including, but not limited to, P_{BT1311}, P_{AM1}, P_{AM2}, P_{AM3}, and P_{AM4}. In some embodiments, the Bacteroides promoter is an ermF gene promoter.

The plasmid expression vector also includes a multiple cloning site (MCS) downstream of the Bacteroides promoter or other bacterial promoter, which may introduce one or more additional restriction sites. Any suitable MCS can be included in the plasmid expression vector. In some embodiments, the MCS introduces one or more additional restriction sites including, but not limited to, KpnI, ClaI, BamHI, SalI, XhoI, StuI, XbaI, SphI and HinDIII. For example, in one embodiment, the MCS is derived from the E. coli vector, pSU20. In certain embodiments, the bacterial gene is inserted or cloned into the MCS via a SphI-XbaI site as discussed below in the working examples.

The plasmid expression vector may also include one or more genes related to replication, expression, or detection of the desired bacterial gene (or portions thereof), including an antibiotic resistance gene (e.g., tetQ, bla), an origin of replication (e.g., rep, repA), or other genes (e.g., mbpA, mbpB, mbpC, metR).

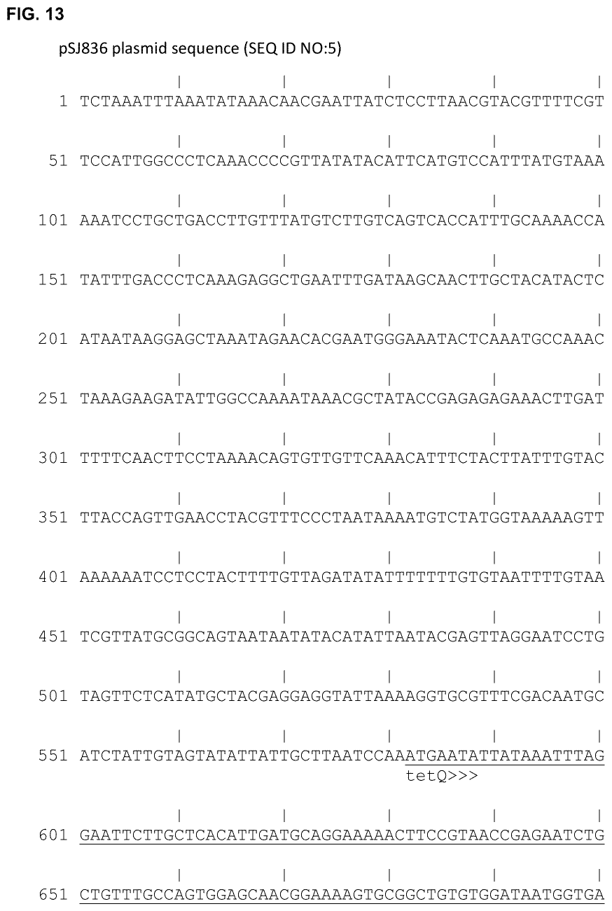

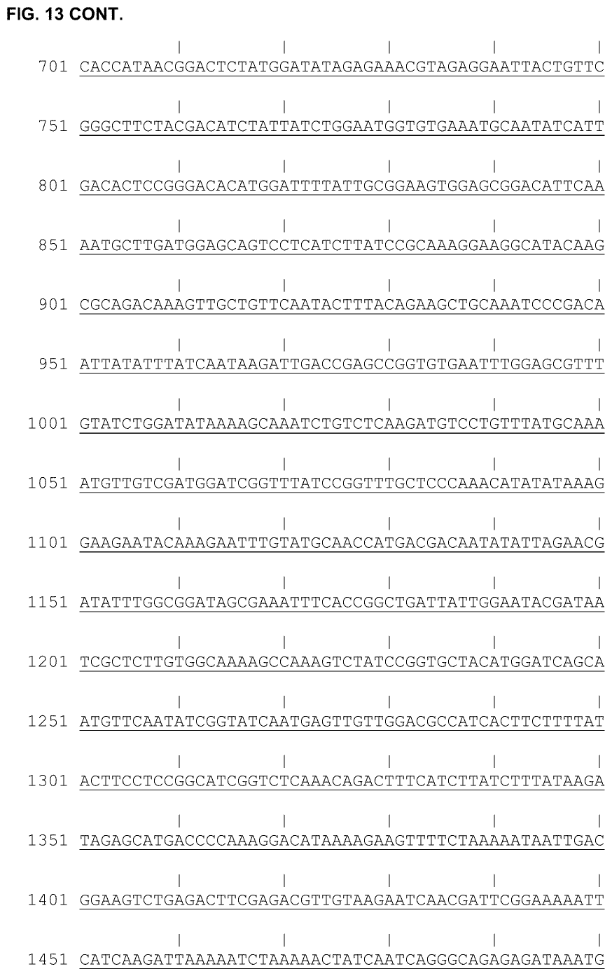

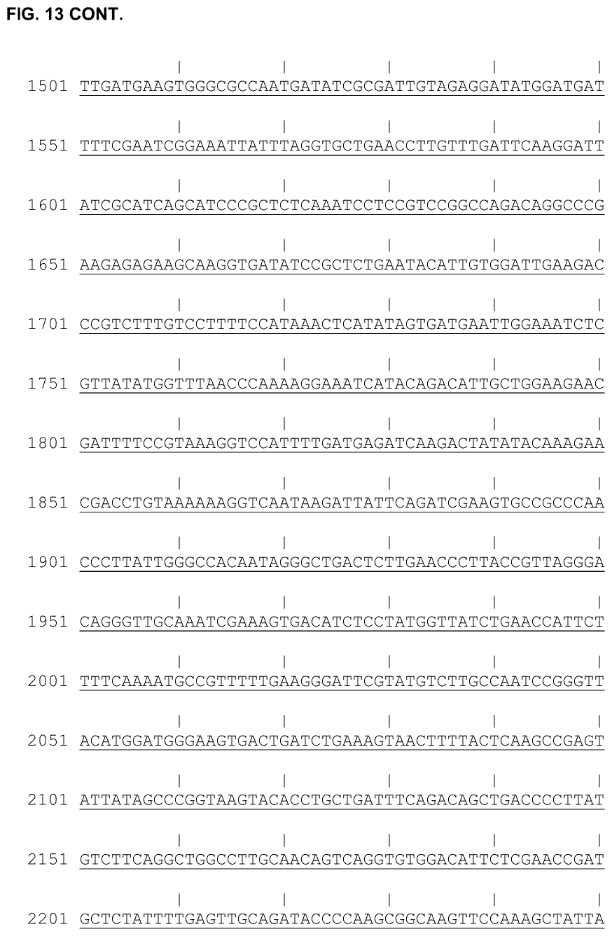

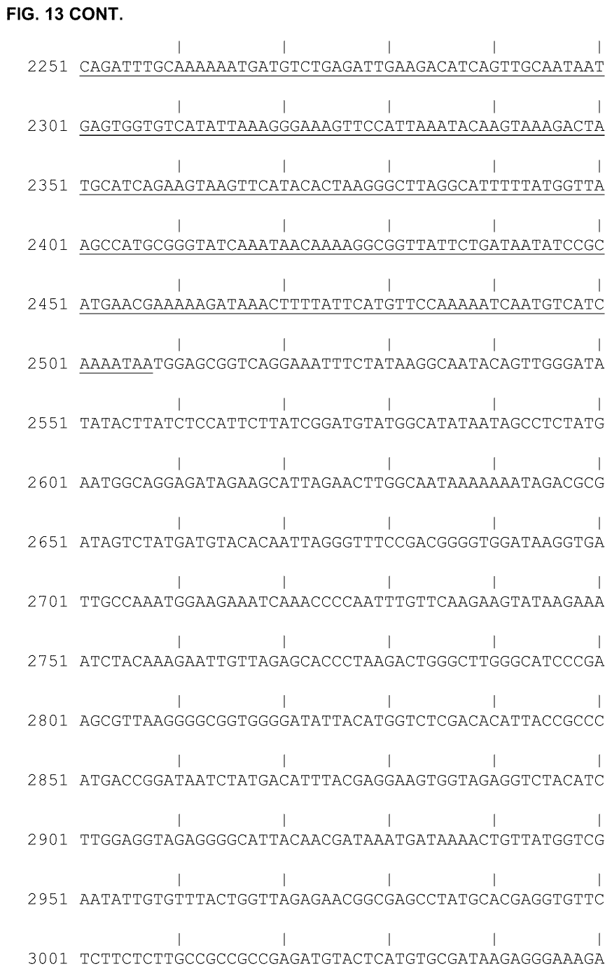

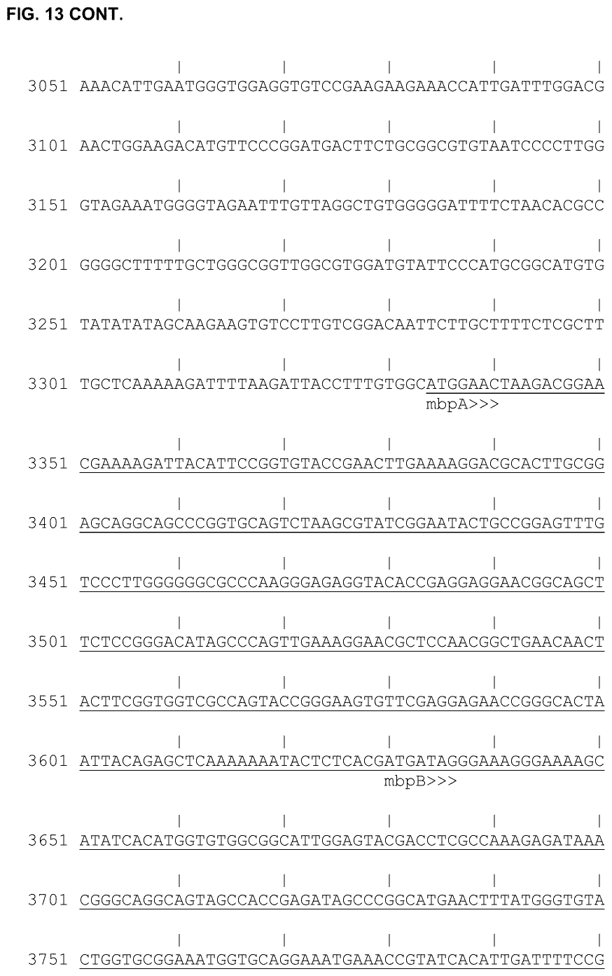

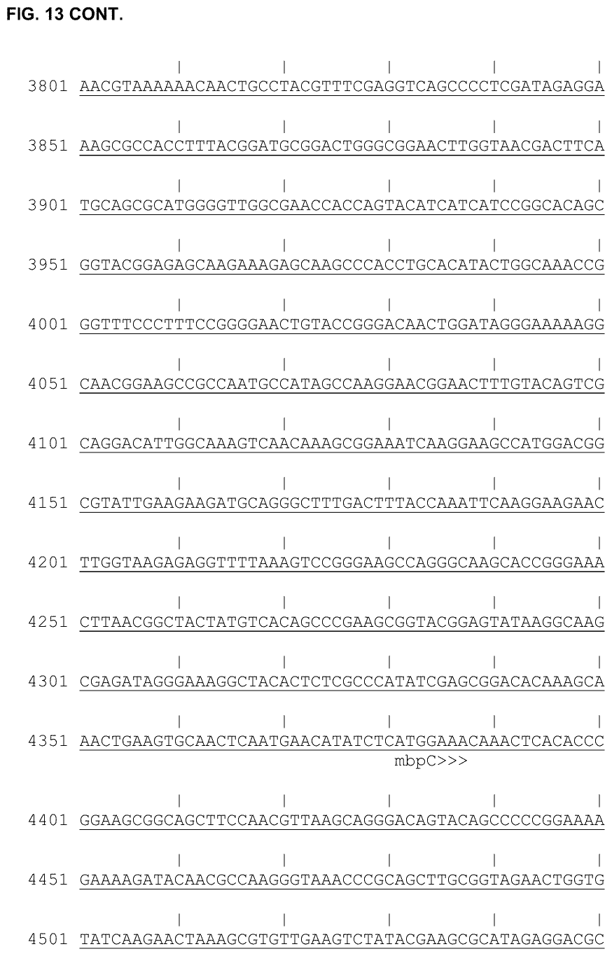

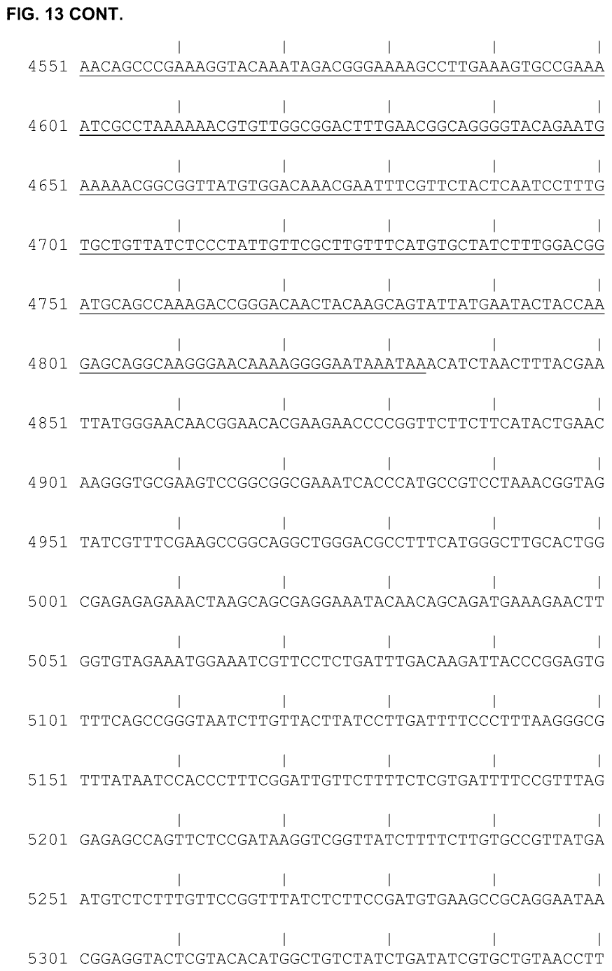

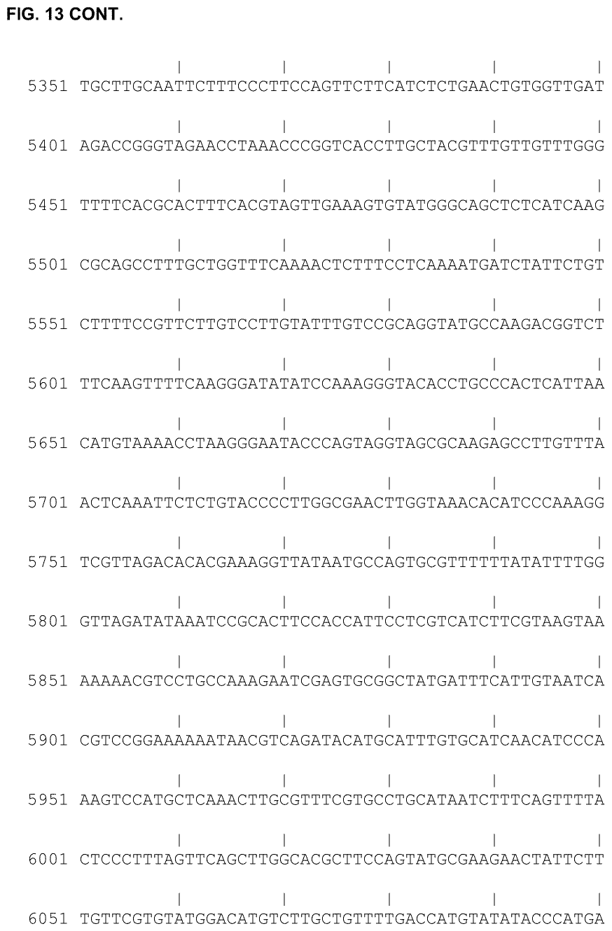

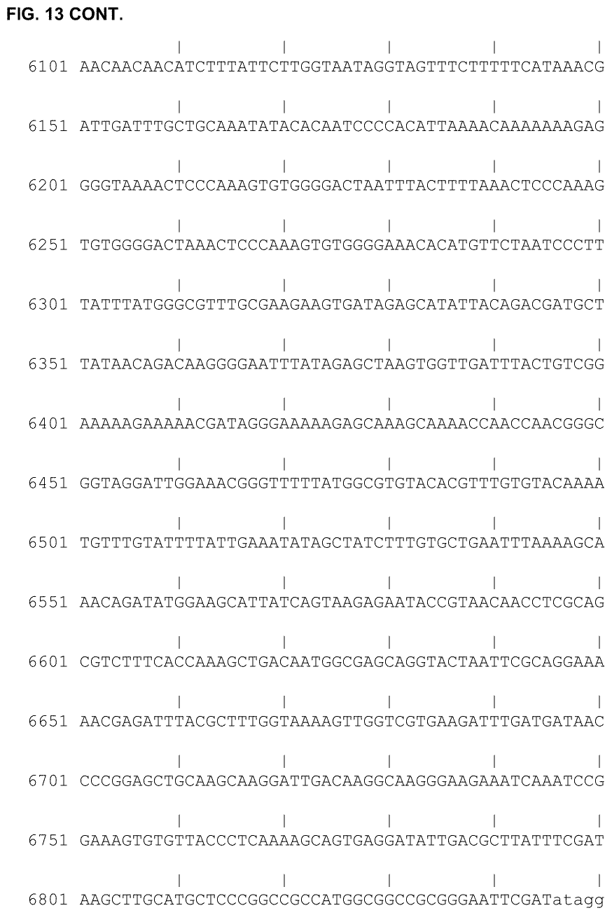

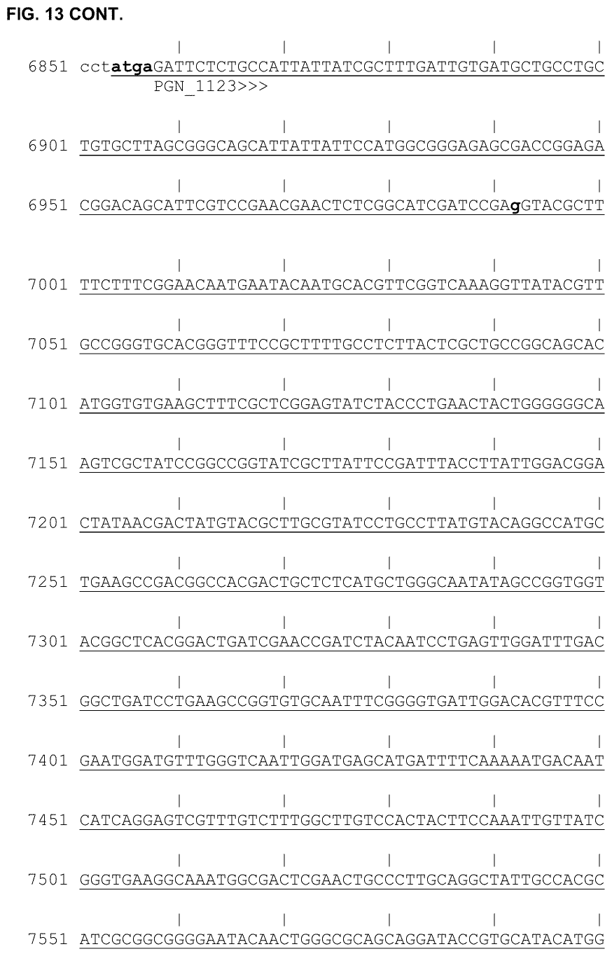

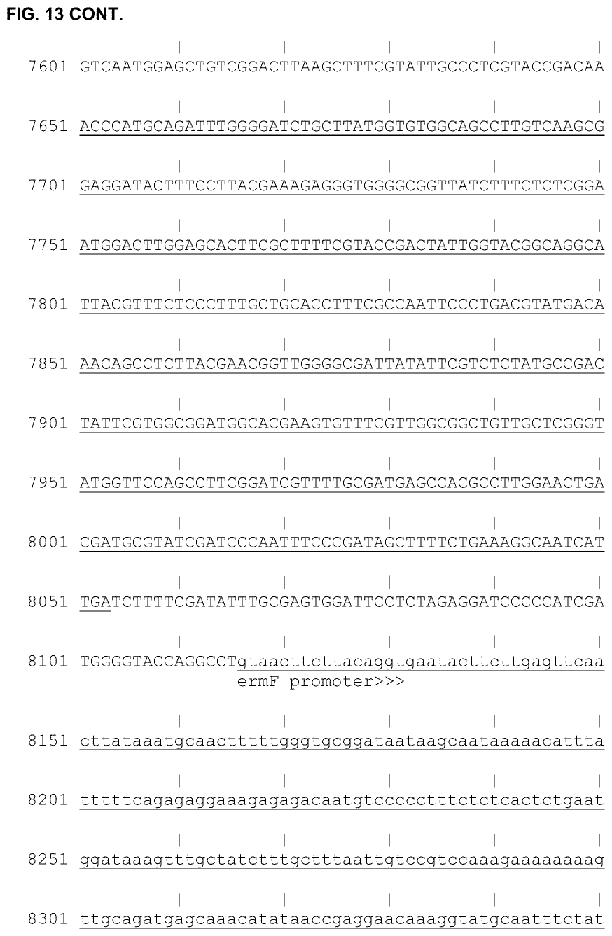

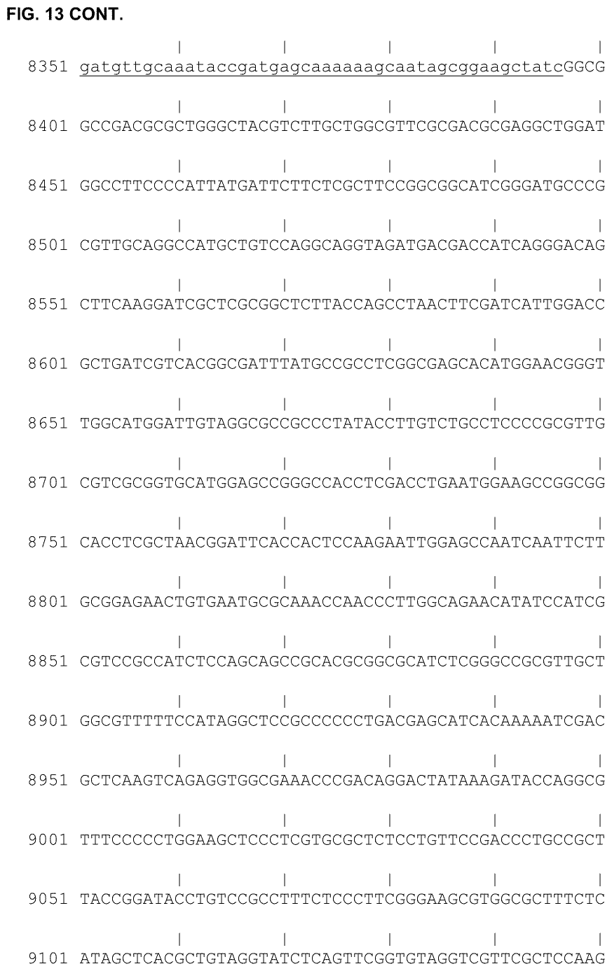

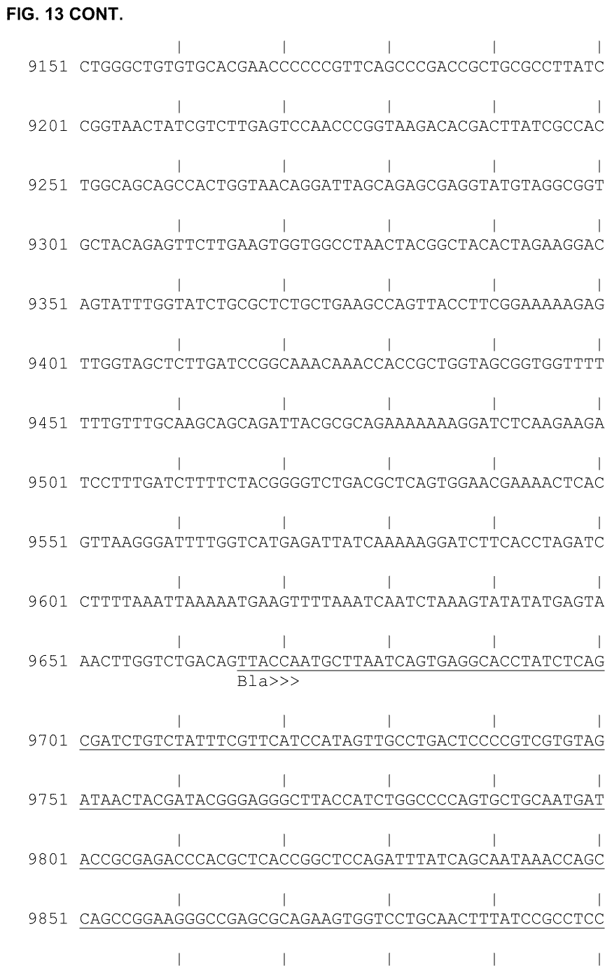

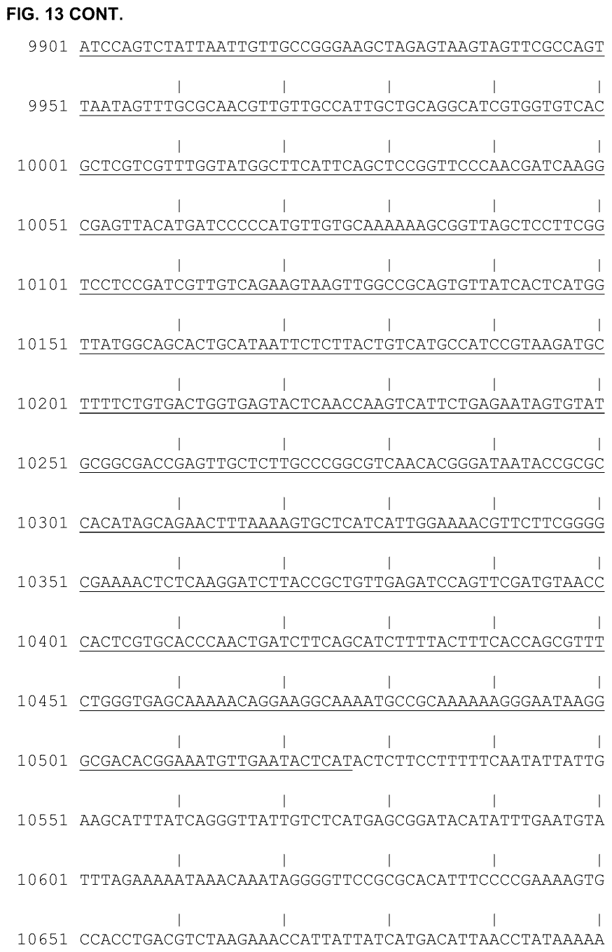

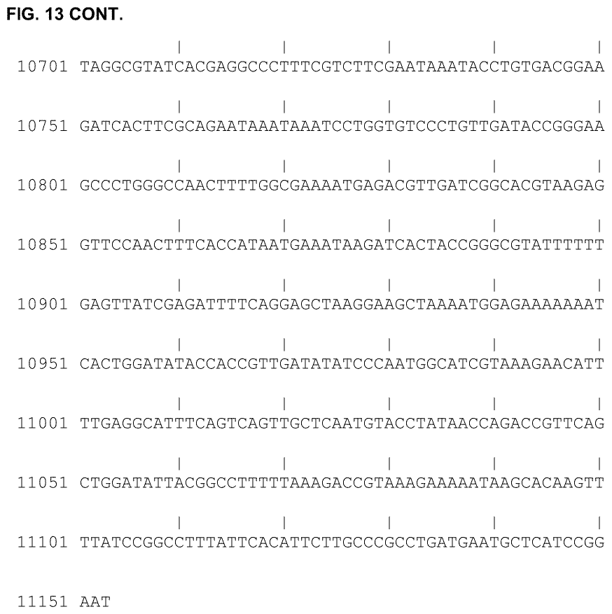

In some embodiments, the plasmid expression vector is a pSJ836 plasmid, which has a nucleotide sequence shown in FIG. 13 (SEQ ID NO: 5).

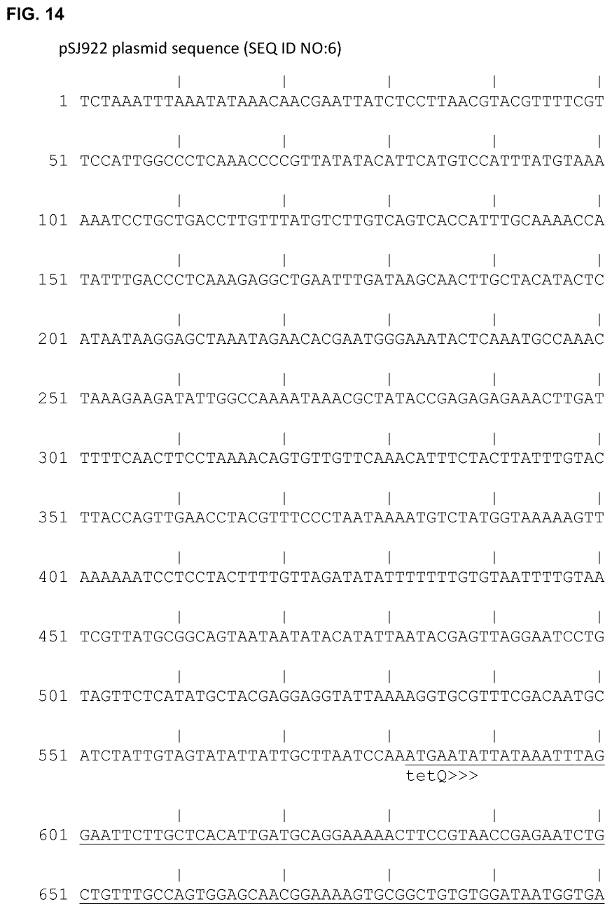

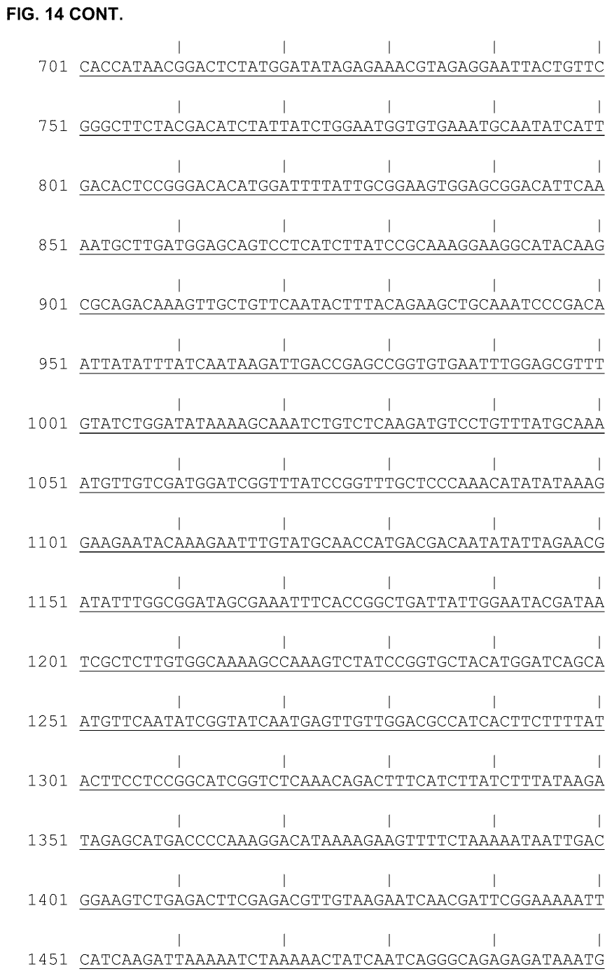

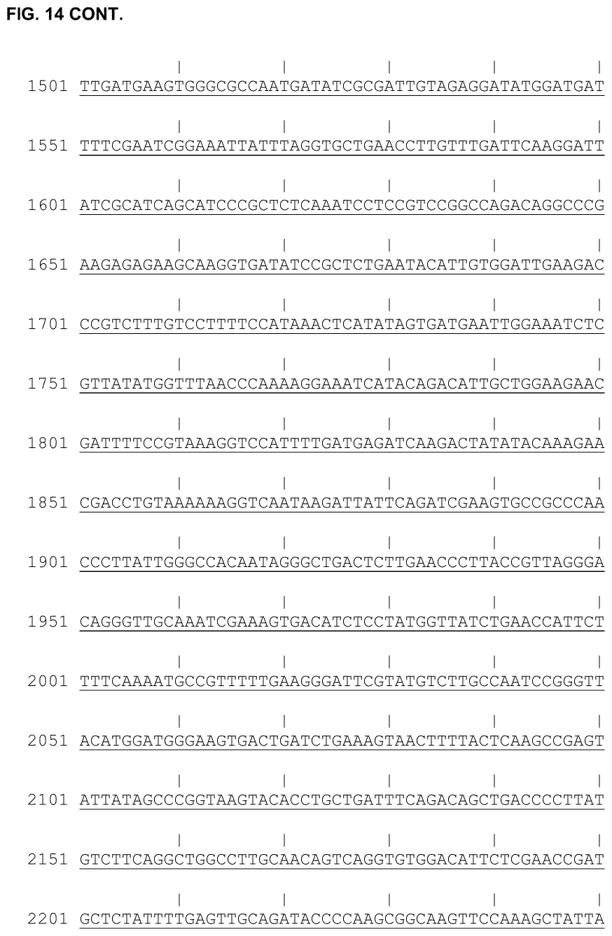

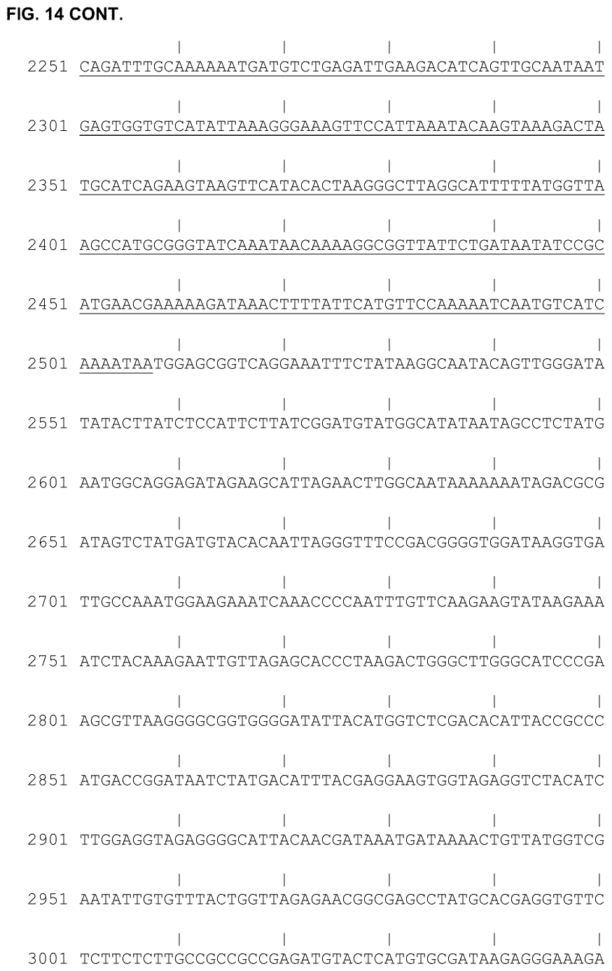

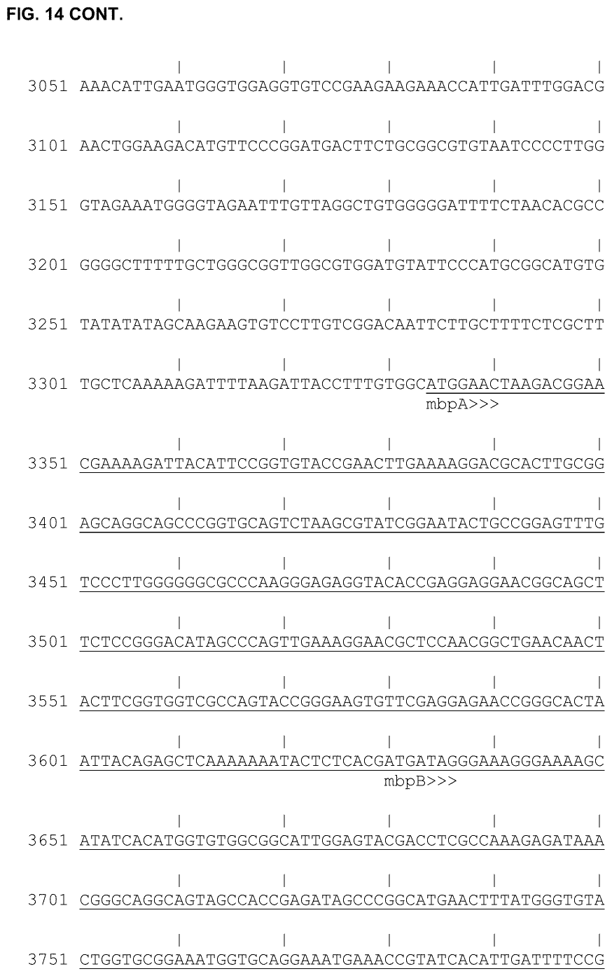

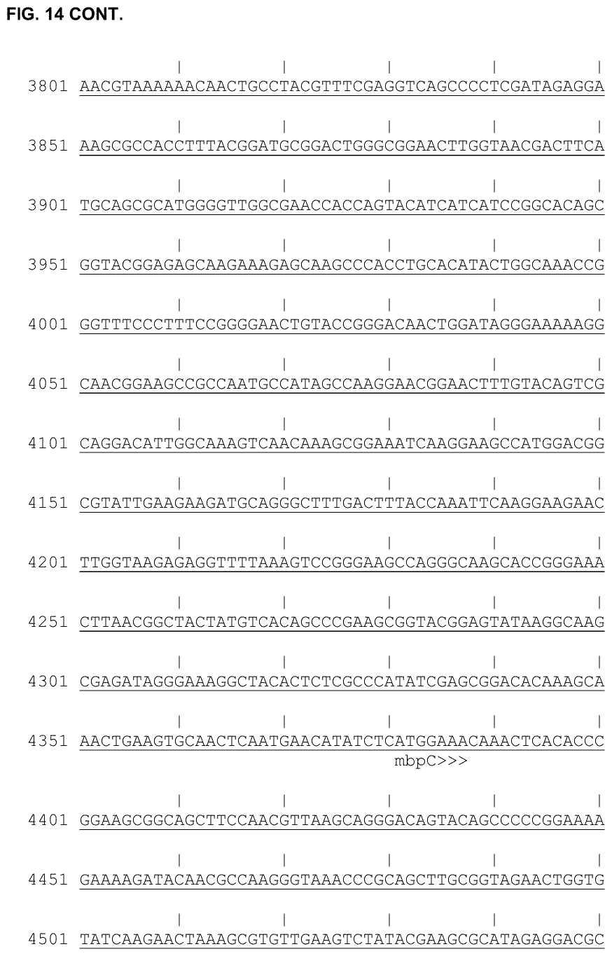

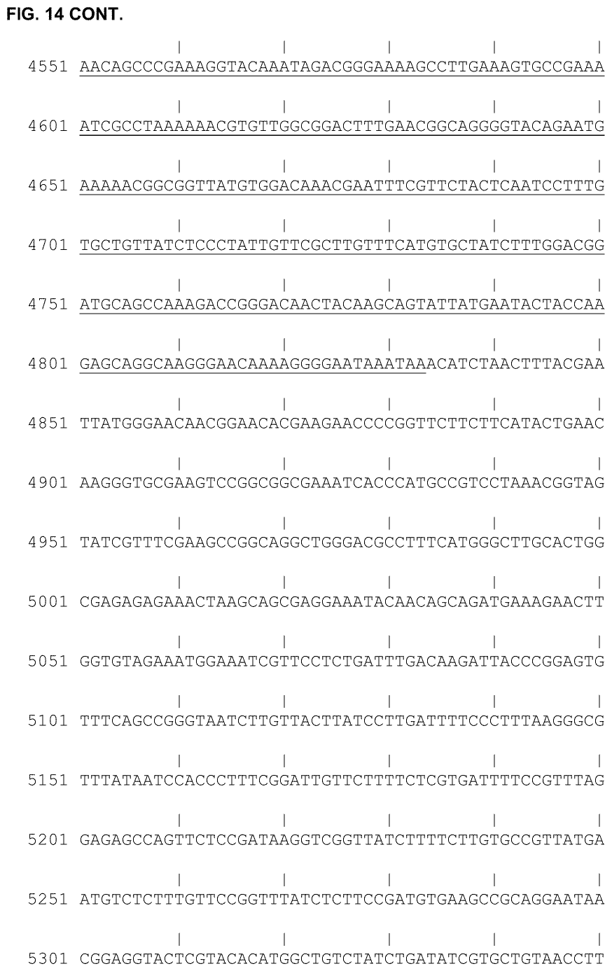

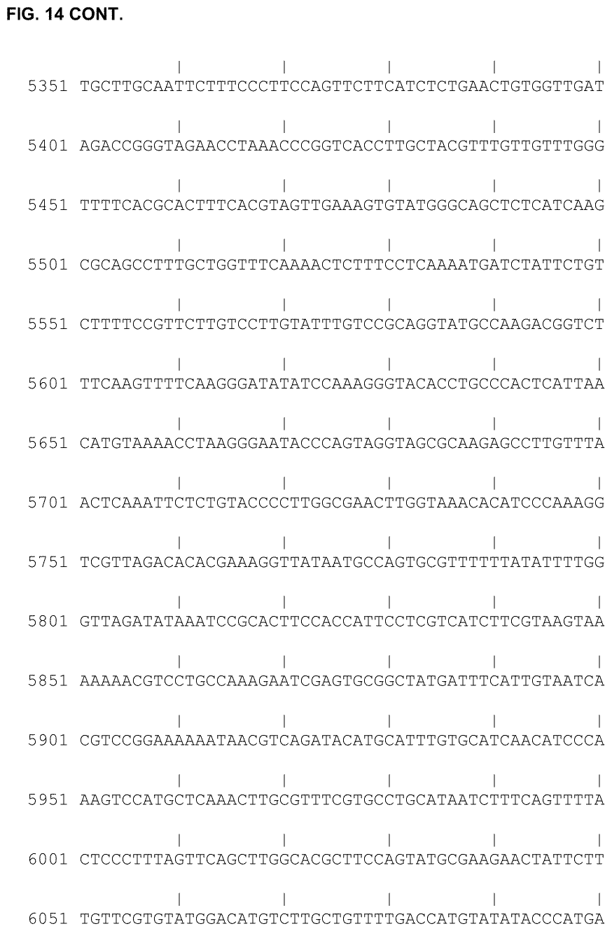

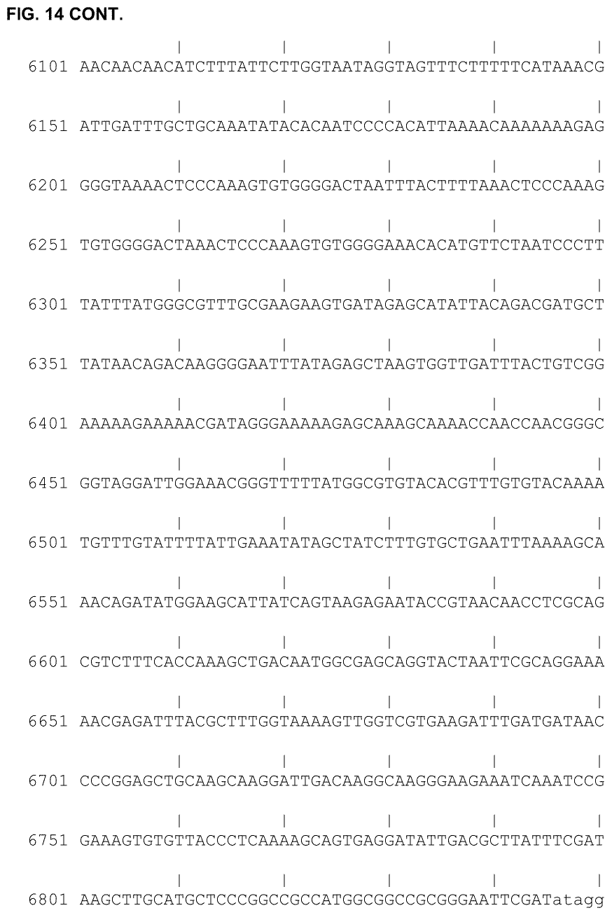

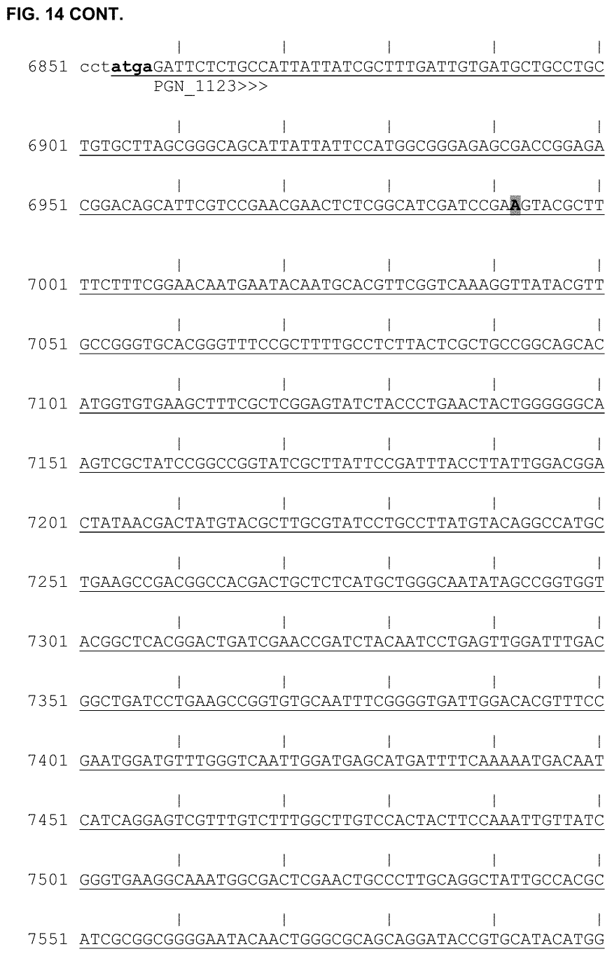

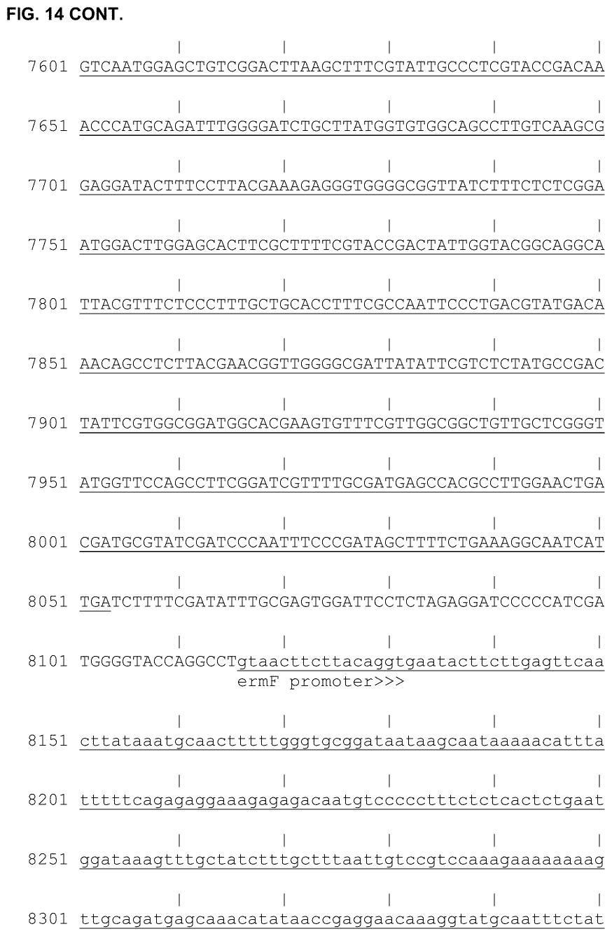

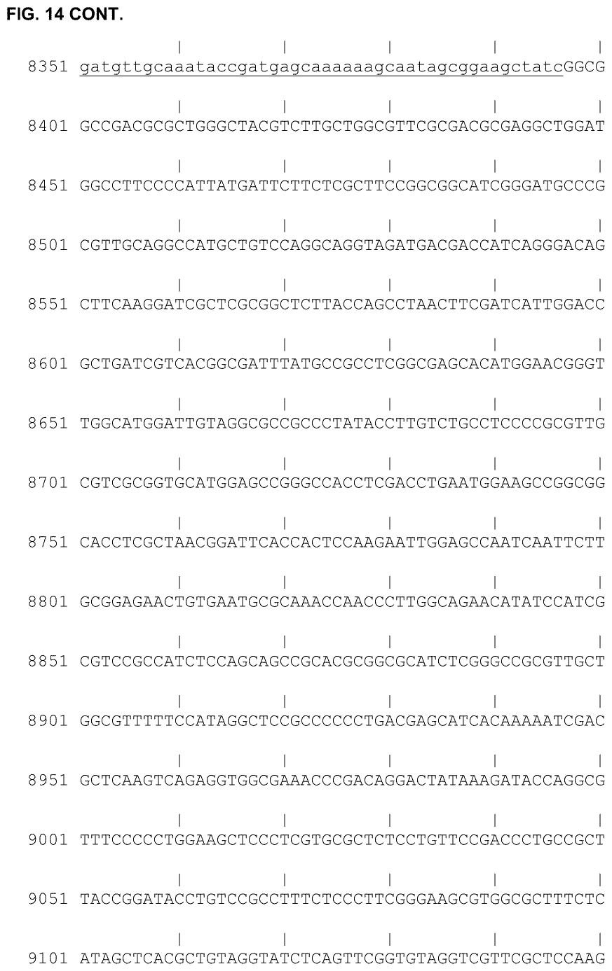

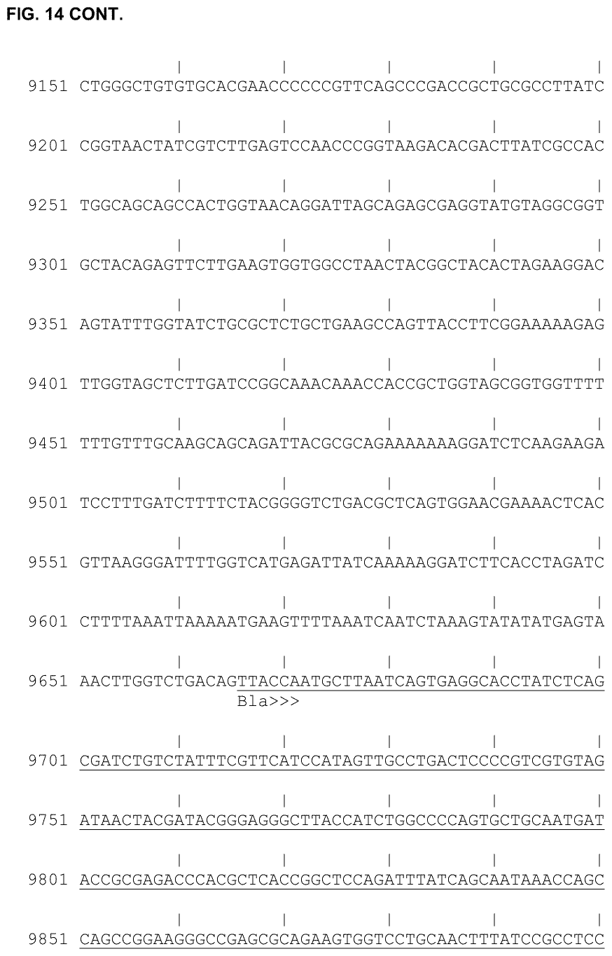

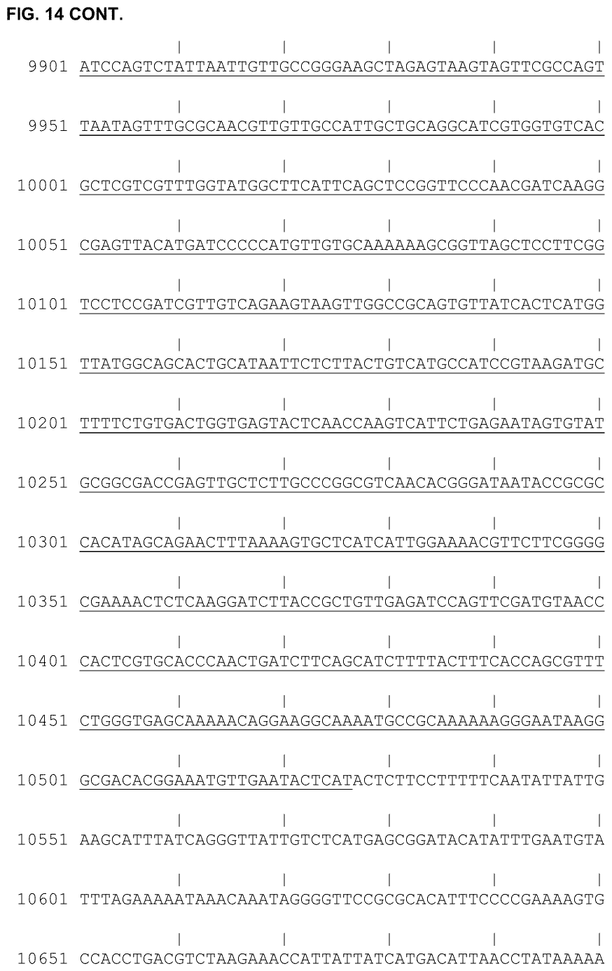

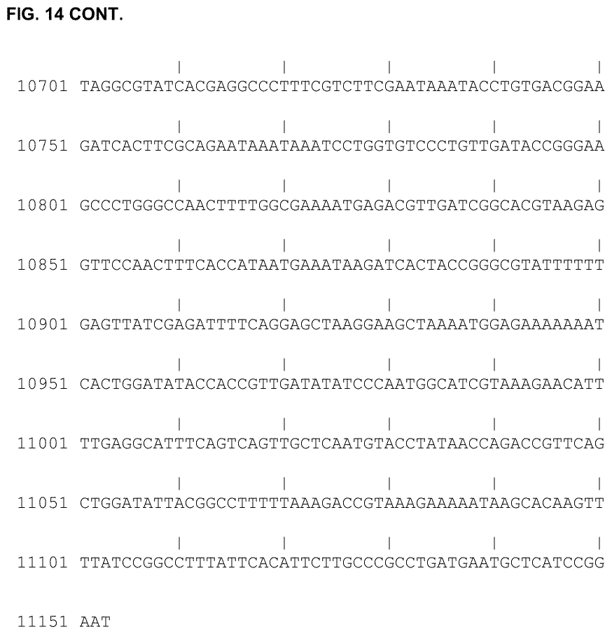

In other embodiments, the plasmid expression vector is a pSJ922 plasmid, which has a nucleotide sequence shown in FIG. 14 (SEQ ID NO: 6).

When the expression vector is transferred to the host cell, the bacterial cell expresses the bacterial gene (e.g., a P. gingivalis lipid A deacylase gene), and that host bacterial cell can be used in culture systems to screen for potential therapeutic compounds. In certain embodiments, the host cell may be a P. gingivalis cell or a B. thetaiotaomicron cell. In one embodiment, the host cell may be a P. gingivalis ΔPGN_1123 mutant containing the plasmid expression vector, pSJ46, and containing plasmid expression vector+deacylase gene. In another embodiment, an expression vector that includes a P. gingivalis lipid A deacylase gene may be transferred to a B. thetaiotaomicron or other Bacteriodes bacterial cell to generate a transformed B. thetaiotaomicron cell that expresses the P. gingivalis lipid A deacylase gene. The transformed B. thetaiotaomicron cell or a population thereof may be used in a cell culture system and in methods or assays to screen for lipid A deacylase inhibitors. Such a method may include steps of contacting a population of bacterial cells transformed with the P. gingivalis lipid A deacylase expression vector with a candidate compound, measuring or determining the TLR4 activity in the transformed B. thetaiotaomicron cells, and identifying whether the candidate compound is a deacylase inhibitor or not. The candidate compound is determined to be a deacylase inhibitor when the TLR4 activity in the population of bacterial cells after contact with the candidate compound is increased as compared to TLR4 activity in the population of bacterial cells before contact with the candidate compound. In one embodiment, the assay is a HEK luciferaseas a detector of activity assay, as described in the working examples below.

This assay can be developed to be robust such that thousands of compounds could be screened in a highly specific manner with robotic technology. One beauty of the screen is that the Pg deacylase gene is highly conserved within the Pg genus so that inhibitor should be highly specific and completely nontoxic. The inhibitor could be added to a mouthwash or toothpaste to prevent or treat chronic pro-inflammatory conditions like periodontitis, among other conditions.

A bacterial cell that expresses the bacterial gene (e.g., a P. gingivalis lipid A deacylase gene) or population thereof can also be used to diagnose a disease or condition related to decreased TLR4 activity, increased lipid A deacylase activity, or increased P. gingivalis presence including, but not limited to, periodontitis.

Additional description of the embodiments and attributes of the methods are described in the working examples below.

From the foregoing, it will be appreciated that specific embodiments of the invention have been described herein for purposes of illustration, but that various modifications may be made without deviating from the scope of the invention. Accordingly, the invention is not limited except as by the appended claims.

### WORKING EXAMPLES

Removal of one acyl chain from bacterial lipid A by deacylase activity is a widely known mechanism used by pathogenic bacteria to evade the host's Toll-like receptor 4 (TLR4)-mediated innate immune response. In Porphyromonas gingivalis, a periodontal pathogen, lipid A deacylase activity converts a majority of the initially synthesized penta-acylated lipid A, a TLR4 agonist, to tetra-acylated structures, which effectively evade TLR4 sensing by being either inert or antagonistic at TLR4.

The gene encoding the lipid A deacylase, essential for P. gingivalis' ability to evade TLR4 sensing, has, however, remained unidentified. Two known bacterial lipid A deacylase-encoding genes are pagL and IpxR, both identified first in Salmonella typhi sv. Typhimurium (34, 35). These two deacylases are distinct from each other both in their primary sequence and in their modes of action, hydrolyzing a C3-chain and C3′-acyloxyacyl pair of acyl chains respectively. A comprehensive search for homologs of pagL and IpxR failed to identify the lipid A deacylase gene in P. gingivalis (34-36). pagL and IpxR have orthologs in other bacteria but, interestingly, are all restricted to the Proteobacteria phylum. P. gingivalis PG0027 was earlier reported to encode the lipid A deacylase gene (37), but was ruled out by the recent finding that PG0027 participates in lipid A C1-phosphatase activity (32), and has no effect on deacylase function.

In the studies discussed in the working examples below, it was demonstrated that the lipid A deacylase gene in P. gingivalis 33277 is encoded by PGN_1123. Identification of the gene began with a screen of a transposon mutant library, employing a functional assay developed for identifying novel mutants that elicit a strong host innate immune response. Using this approach, a candidate mutant that demonstrated potent TLR4 activation was identified, consistent with a lipid A deacylase phenotype. An affected gene in the transposon mutant was shown to be required for lipid A deacylation and identified to be PGN_1123. The encoded protein is highly conserved in P. gingivalis, bearing no significant homology to the two known lipid A deacylases, nor to other proteins of known function, indicating PGN_1123 encodes a novel bacterial lipid A deacylase enzyme. In identifying PGN_1123, the studies below show successful identification of the gene that encodes the P. gingivalis lipid A deacylase enzyme.

PGN_1123 is highly conserved within P. gingivalis, and putative orthologs are phylogenetically restricted to the Bacteroidetes phylum. Lipid A of ΔPGN_1123 mutants is penta-acylated, devoid of tetra-acylated structures, and the mutant strain provokes a strong TLR4-mediated pro-inflammatory response, in contrast to the negligible response elicited by wild-type P. gingivalis. Heterologous expression of PGN_1123 in Bacteroides thetaiotaomicron promoted lipid A deacylation, confirming PGN_1123 encodes the lipid A deacylase enzyme. Identification of PGN_1123 and its expression in bacteria has also led to the development of an assay to identify inhibitors of lipid A deacylase to treat P. gingivalis infection.

### Example 1

### Identification of P. gingivalis Mutants that Stimulate the Human Innate Immune Response

Materials and Methods

Bacterial strains and growth conditions. P. gingivalis 33277, Bacteroides thetaiotaomicron ATCC 29148, and E. coli DH10b were obtained internally. E. coli SM10λpir (pSAM_Bt) was obtained from Dr. Andrew Goodman's laboratory (43). P. gingivalis strains were grown on blood agar plates containing 5% sheep's blood, and in TYHK broth (30 g/liter Trypticase soy broth, 5 g/liter yeast extract, and 1 mg/liter vitamin K3). Following sterilization by autoclaving, filter-sterilized hemin was added to TYHK broth, just prior to inoculation, to a final concentration of 1 μg/ml. 10 μg/ml hemin was added for growth in high hem in conditions. TYHK agar plates were used for growth of P. gingivalis on solid medium as well. Hemin (1 μg/ml) and antibiotics were added following sterilization. Antibiotics were added to the following concentrations: erythromycin (erm), 5 μg/ml; tetracycline (tet), 0.5 μg/ml. E. coli strains were grown in Luria broth (10 g/liter tryptone, 5 g/liter yeast extract, 5 g/liter NaCl); 100 μg/ml ampicillin was added when required for selection. B. thetaiotaomicron was grown on TYHK agar plates and TYHK broth containing 1 μg/ml hemin. All anaerobic strains were grown in an anaerobic growth chamber (5% H₂, 5% CO₂, 90% N₂) at 37° C.

Transposon mutagenesis. Conjugation was used to transfer the mariner transposon-containing plasmid pSAM_Bt from E. coli SM10λpir to P. gingivalis 33277. pSAM_Bt encodes the (a) RP4 origin of transfer, required for mobilization by bi-parental mating, (b) mariner transposon encompassing an ermG cassette that confers erythromycin resistance, and (c) transposase encoding gene, required for transposition. 100 mls of P. gingivalis 33277 and 25 mls of SM10λpir (pSAM_Bt) were grown to mid-log phase to an OD600 of ˜1.0 and ˜0.5 respectively. After pelletting and re-suspending each culture in 1 ml TYK broth, the two cultures were mixed, 100 μl aliquots spotted on blood agar plates, and incubated overnight aerobically. The bacterial mix from 8-10 spots were transferred using sterile swabs into 10 mls PBS, spun and re-suspended in 1.5 mls TYK broth. 100 μl aliquots were plated on TYHK plates containing 5 μg/ml erythromycin, for selection of transposon containing 33277 strains, and 50 μg/ml gentamycin, for counter-selection of E. coli. Transposon containing erythromycin and gentamycin resistant 33277 colonies were seen after 4-5 days of anaerobic incubation.

Transposon mutant candidate colonies were patched on TYHK Erm Gm plates at 48 colonies/plate. After 3-4 days of growth, they were inoculated into 150 μl TYHK Erm media in 96-well plates. Two days later they were used for infection of MM6 cells.

IL-6 production from Monomac 6 (MM6) cells. MM6 cells were grown in media comprising RPMI 1640 supplemented with 10% heat-inactivated fetal bovine serum, 1% penicillin-streptomycin, 1% non-essential amino acids and 1% OPI media supplement. 2.5×105 cells were seeded in 96 well plates in 200 μl aliquots. 24 h later, the cells were infected with 20 μl of each Tn mutant that had been grown for 2 days in the 96-well holding plate. 24 h after infection, the cells were spun down in the 96-well plate, and the amount of IL-6 in 100 μl supernatant was measured by ELISA according to manufacturer's instructions (eBioscience).

Preparation of LPS and isolation of lipid A. Bacteria were cultured for 48 h in TYHK medium containing 1 μg/ml hemin, or in 10 μg/ml hemin for growth in high hemin conditions. LPS was isolated from 150 ml culture using the Tri-reagent protocol as previously described (44). To generate lipid A, dried LPS samples were re-suspended in 10 mM sodium acetate [pH 4.5] containing 1% sodium dodecyl sulfate. The solution was heated for 1 h at 100° C. followed by lyophilization overnight. The resulting dried lipid A was re-suspended in ice-cold 95% ethanol containing 0.02 N HCl, spun, washed three times in 95% ethanol, and subjected to a final extraction with 1160 μl of chloroform-methanol-water (1:1:0.9), a bi-phasic solution that separated residual carbohydrate contaminants into the aqueous phase. The chloroform layer containing the lipid A was dried in a fume hood and used for MALDI-TOF MS analyses.

MALDI-TOF mass spectrometry. Lipid A samples were dissolved in 10 μl norharmane matrix, prepared by adding 10 mg norharmane to 1 ml of 1:1 chloroform:methanol solution and mixing well. 1 μl of each sample was analysed in both positive and negative ion modes on an AutoFlex II Analyzer (Bruker Daltonics). Data were acquired with a 50 Hz repletion rate and up to 3000 shots were accumulated for each spectrum. Instrument calibration and all other tuning parameters were optimized using HP Calmix (Sigma-Aldrich). Data was acquired and processed using Flex Analysis software (Bruker Daltonics).

HEK TLR2 and TLR4 assays. The assays were performed as previously described (44). Briefly, HEK293 cells were plated in 96-well plates, and transfected the following day with plasmids encoding human TLRs, NF-κB-dependent firefly luciferase reporter and β-actin promoter-dependent Renilla luciferase reporter. In the case of human TLR4, 0.002 μg plasmid encoding human TLR4 was co-transfected with 0.0025 μg plasmid encoding human MD-2. In the case of human TLR2, 0.001 μg plasmid encoding TLR2 was co-transfected with 0.002 μg plasmid encoding human mCD14. 18 to 20 h post-transfection, test wells were stimulated in triplicate for 4 h at 37° C. with various doses of sample, which were suspended in Dulbecco's modified Eagle medium (DMEM) containing 10% human serum. For stimulation with intact bacteria, 1 ml cultures of the indicated strains were first washed with TYHK, and their concentration estimated by measuring the optical density at 600 nm. Luciferase activity was assayed using a dual luciferase assay reporter system (Promega, Madison, Wis.). NF-κB activity was measured as the ratio of NF-κB-dependent firefly luciferase activity to β-actin promoter-dependent Renilla luciferase activity, which served as an internal standard. The data were plotted as the fold difference of NF-κB activity of the sample over unstimulated control.

Nested PCR to identify transposon insertion sites. Semi-random primer Arb1 (78) was used with a primer reading outwards from mariner transposon, SamSeq2 (43), to amplify the transposon-chromosome junction by polymerase chain reaction (PCR). Several fragments obtained were gel extracted together and used as template for a nested PCR product using primers Arb2 (same as Arb1 but lacking the semi-random N nucleotides) and SamSeq2, a primer reading outwards and closer to the edge of the transposon than SamSeq1. A prominent 650 bp PCR product was obtained which was cleaned and sent for Sanger sequencing at Genewiz.

Whole genome sequencing and analysis. Genomic DNA was purified from P. gingivalis cultures using the DNeasy Blood & Tissue kit (Qiagen). Genomic DNA from wild-type 33277 and the transposon mutant, J5-c5, were subjected to paired-end, whole-genome sequencing on IIlumina's MiSeq platform.

Transposon insertion sites in J5-c5 were identified by aligning reads spanning junction sites between genomic sequence and the terminii of the transposon. The reads were first trimmed and filtered based on quality analysis by FastQC. Next, they were aligned against the P. gingivalis 33277 reference genome using Bowtie2, and un-alignable reads aligned against the sequence of the mariner transposon. The ratio of whole genome coverage to mariner Tn coverage in J5-c5 was 4.4, suggesting there were at least four, and perhaps five, transposon insertions. Reads that could not be aligned strictly within 33277 reference genome or mariner Tn sequence were assumed to be enriched in junction spanners. These reads were searched for the terminii of the mariner Tn, and reads confirmed to be positive for Tn terminus were subjected to BLAST analysis, from which genomic sequences flanking the insertion could be inferred. The precise insertion sites inferred by this method were verified to be as predicted by amplifying the junction site using PCR and sequencing the PCR product.

RNAseq. RNA was purified from P. gingivalis cultures that were treated with Trizol (Invitrogen) to facilitate cell lysis. Briefly, a fully grown 3 ml culture was spun and the pellet brought up in 900 μl Trizol, mixed with 200 μl chloroform and the sample spun for 15 minutes. The upper aqueous layer was transferred to a new tube, an equal volume of isopropanol added, mixed well and frozen in a −80 C freezer. The next day, the samples were thawed, spun, pellet washed with 70% ethanol, and re-suspended in water. 20 μg of this sample, comprising crude RNA, was subjected to DNase treatment using RQ1 DNase (Promega). The sample was again subjected to Trizol and chloroform treatment, spun and the upper aqueous layer was concentrated using Zymo Clean and Concentrator kit (Zymo Research), according to manufacturer's instructions. RNAseq was performed on three independent biological samples of 33277 and J5-c5 RNA using Illumina's HiSeq platform at the University of Washington Center for Precision Diagnostics.

RNAseq data was analyzed closely following guidance outlined in Law et al (45).

Construction of P. gingivalis 33277 mutants. Deletion mutations were constructed in P. gingivalis 33277 by replacing the gene of interest with either an erythromycin resistance cassette, ermF, or tetracycline resistance cassette, tetQ. Flanking sequences of the gene to be deleted, ˜500-700 base-pairs upstream and 500-700 base-pairs downstream respectively, were amplified by PCR using genomic DNA from P. gingivalis 33277 as template. The primers used for the up-flank, ‘a’ and ‘b’, and for the down-flank, ‘c’ and ‘d’, are listed in Table 1 below for ΔPGN_1123::erm, ΔPGN_1124::erm and Δkgp::erm deletion constructs, and for replacement of Tn-1 with tetQ in J5-c5.

TABLE 1
Primers used in this study.

| nucle- | Specific | Orien- | |
|---|---|---|---|
| otide | to | tation | Sequence* |
| Δ1124-a | PGN_1124 | forward | AGACTCAATAGCAGA |
| | up-flank | | TCGACG |
| | | | (SEQ ID NO: 8) |
| Δ1124-b | PGN_1124 | reverse | TTCATACCGCCCAAG |
| | up-flank | | gatCCTGTTGCTGTC |
| | | | ATA |
| | | | (SEQ ID NO: 9) |
| Δ1124-c | PGN_1124 | forward | TATGACAGCAACAGG |
| | down- | | atcCTTGGGCGGTAT |
| | flank | | GAA |
| | | | (SEQ ID NO: 10) |
| Δ1124-d | PGN_1124 | reverse | GACCCAAACATCCAT |
| | down- | | TCGG |
| | flank | | (SEQ ID NO: 11) |
| Δ1124- | PGN_1124 | reverse | TCTCATACATGGCAC |
| confirm | | | GCAT |
| | | | (SEQ ID NO: 12) |
| Δ1123-a | PGN_1123 | forward | ATGCGTGCCATGTAT |
| | up-flank | | GAGA |
| | | | (SEQ ID NO: 13) |
| Δ1123-b | PGN_1123 | reverse | GGACTGTCAAAGGAG |
| | up-flank | | gatcCTAAGCACAGC |
| | | | AGG |
| | | | (SEQ ID NO: 14) |
| Δ1123-c | PGN_1123 | forward | CCTGCTGTGCTTAGg |
| | down- | | atcCTCCTTTGACAG |
| | flank | | TCC |
| | | | (SEQ ID NO: 15) |
| Δ1123-d | PGN_1123 | reverse | CCGTAACCGGGTACG |
| | down- | | AT |
| | flank | | (SEQ ID NO: 16) |
| Δ1123- | PGN_1123 | reverse | GACCCAAACATCCAT |
| confirm | | | TCGG |
| | | | (SEQ ID NO: 17) |
| Δkgp-a | kgp up- | forward | atccatggACGCCCG |
| | flank | | ATACCCATACTC |
| | | | (SEQ ID NO: 18) |
| Δkgp-b | kgp up- | reverse | CACAAAGTCTCCGAG |
| | flank | | TggatccATTCAGAG |
| | | | AACCACG |
| | | | (SEQ ID NO: 19) |
| Δkgp-c | kgp | forward | CGTGGTTCTCTGAAT |
| | down- | | ggatccACTCGGAGA |
| | flank | | CTTTGTG |
| | | | (SEQ ID NO: 20) |
| Δkgp-d | kgp | reverse | tatgtcgacAACAGG |
| | down- | | TTGTCCGTCAGC |
| | flank | | (SEQ ID NO: 21) |
| Δkgp- | kgp | reverse | AACTTCCTAACTGCT |
| confirm | | | GGCAC |
| | | | (SEQ ID NO: 22) |
| ΔTn1-a | Tn1 up- | forward | AAGGCTTGATGCTGA |
| | flank | | AGACC |
| | | | (SEQ ID NO: 23) |
| ΔTn1-b | Tn1 up- | reverse | GTCATTTCTTATTAA |
| | flank | | GAATAggatccTATT |
| | | | TACGTTGCGAGC |
| | | | (SEQ ID NO: 24) |
| ΔTn1-c | Tn1 | forward | GCTCGCAACGTAAAT |
| | down- | | AggatccTATTCTTA |
| | flank | | ATAAGAAATGAC |
| | | | (SEQ ID NO: 25) |
| ΔTn1-d | Tn1 | reverse | TCTTTGCCGGCATCT |
| | down- | | TTGC |
| | flank | | (SEQ ID NO: 26) |
| 1123- | PGN_1123 | forward | ataggcctATGAGAT |
| start | | | TCTCTGCCATTATTA |
| | | | TCG |
| | | | (SEQ ID NO: 27) |
| 1123- | PGN_1123 | reverse | attctaGAGGAATCC |
| stop | | | ACTCGCAAATATC |
| | | | (SEQ ID NO: 28) |
| SJ59 | ermF | forward | ggaattcggccgCCG |
| | promoter | | ATAGCTTCCGCTATT |
| | | | G |
| | | | (SEQ ID NO: 29) |
| SJ60 | ermF | reverse | taggtaccaggcctG |
| | promoter | | TAACTTCTTACAGGT |
| | | | GAATAC |
| | | | (SEQ ID NO: 30) |

*Note:
Nucleotides in lowercase letters refer to bases that add or complete a restriction site.

The ‘b’ and ‘c’ primers were designed to be overlapping primers that are reverse complements of each other. The forward primer ‘c’ comprised 15 bp of the end of the up-flank, an XbaI restriction site, and 15 bp of the beginning of the down-flank. The upstream and downstream flanking fragments were first amplified separately using ‘a-b’ and ‘c-d’ primer sets respectively. The two products were cleaned, mixed together, and outer primers ‘a-d’ used to amplify the aligned product that comprised both flanks. The amplified flanks were ligated by TA cloning into pGEM-T Easy, a narrow host range vector that can propagate in E. coli but is a suicide vector in P. gingivalis. Finally, an ermF or tetQ cassette was introduced between the flanks into the XbaI site to complete each deletion construct.

P. gingivalis 33277 deletion mutants were generated by introducing each deletion plasmid into P. gingivalis 33277 by natural transformation. Briefly, 0.5×10⁹ bacteria were mixed with 1 μg of the deletion plasmid, incubated overnight in a loosely fastened screw-cap eppendorf tube in the anaerobic chamber, and plated the next morning on TYHK agar plates containing the appropriate antibiotic. Putative mutant colonies that arose by homologous recombination, entailing a double cross-over event of the flanking regions into the chromosome, were seen 5-6 days later. Loss of the targeted gene in the deletion mutant was confirmed by PCR analysis using primers designed to detect the coding sequences (Table 1).

Accession number. RNAseq data obtained by next generation sequencing of wild-type 33277 and the J5-c5 transposon mutant, performed on three biological replicates, is available in the NCBI GEO database under accession number GSE122289.

Development of a Cell Based Functional Screen to Identify P. gingivalis Mutants that Stimulate the Human Innate Immune Response

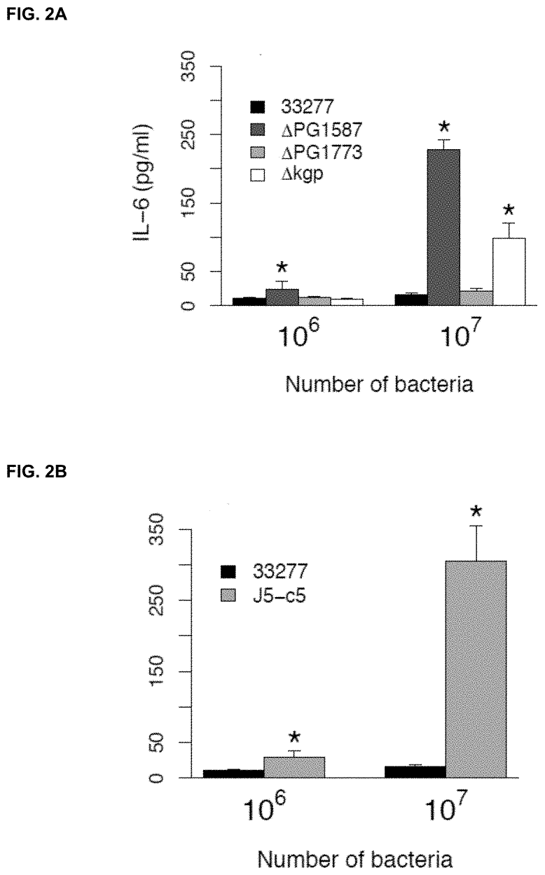

To identify novel genetic determinants that play a role in conferring to P. gingivalis its wide ranging ability to evade immune recognition, a P. gingivalis mutant library was screened for mutants that stimulate a high immune response. Monomac 6 (MM6) cells, a human monocyte cell line, were tested to serve as an in vitro model. Infection of MM6 cells with wild-type P. gingivalis 33277 did not induce secretion of the pro-inflammatory cytokine, IL-6, in the supernatant, as measured by ELISA (enzyme linked immuno-sorbent assay) (FIG. 2A). This is consistent with the observations regarding P. gingivalis' ability to effectively evade or dampen immune defense mechanisms (38-40). Infection with a 33277 ΔPG1587 mutant, which possesses TLR4-stimulating penta-acylated lipid A in its LPS, on the other hand, stimulated IL-6 secretion. This was in contrast to infection with a ΔPG1773 mutant, which harbors anti-inflammatory tetra-acylated antagonistic lipid A. The gingipain proteases, Kgp and Rgp, are also known to dampen innate immune responses by, for example, degrading CD14 (41, 42), a co-receptor of TLR2 and TLR4 signaling complexes. Infection of MM6 cells with a 33277 Δkgp mutant, indeed, led to a higher IL-6 response. The MM6/IL-6 assay, hence, was validated as a sensitive model system for detecting increased immune stimulatory activity by P. gingivalis mutants that is conferred by functional loss of an immune-modulating gene.

Isolation of a P. gingivalis Transposon Mutant that Stimulates a Vigorous Pro-inflammatory Response

A mariner transposon (Tn) isolated from Bacteroides thetaiotaomicron, a close relative of P. gingivalis, was used to create a P. gingivalis 33277 mutant library (43). Each round of mutagenesis yielded hundreds of mutants, obtained by selecting for erythromycin resistance conferred by the Tn-encoded erythromycin resistance cassette, ermG. The Tn mutants were grown individually in 96-well plates for two days, at which point most had grown to visibly high turbidity. MM6 cells, also grown in 96-well plates, were infected with the mutant library, and screened for IL-6 secretion by ELISA. The vast majority of ˜1000 mutants screened exhibited non-detectable levels of IL-6 in the supernatant, similar to the wild-type (WT) strain. A few mutants, ˜5-10%, exhibited a pronounced IL-6 response. A vast majority of the ‘positive hit’ mutants, however, were slow growing strains, as was apparent by sight in the 96-well Tn mutant-holding plate. In this case, slow growing mutants were likely stimulating an increased inflammatory response due to low level of expression of immune-suppressing proteases such as Kgp on the bacterial surface. The putative positive hit mutants were re-screened, and obtained one candidate, named J5-c5, which (a) displayed normal growth, similar to WT, and (b) stimulated IL-6 production, unlike WT (FIG. 2B). These two features are characteristic phenotypes of ΔPG1587 and Δkgp mutants as well.

The J5-c5 Mutant Stimulates TLR4-Dependent Signaling, and Fails to Produce Tetra-Acylated Lipid A

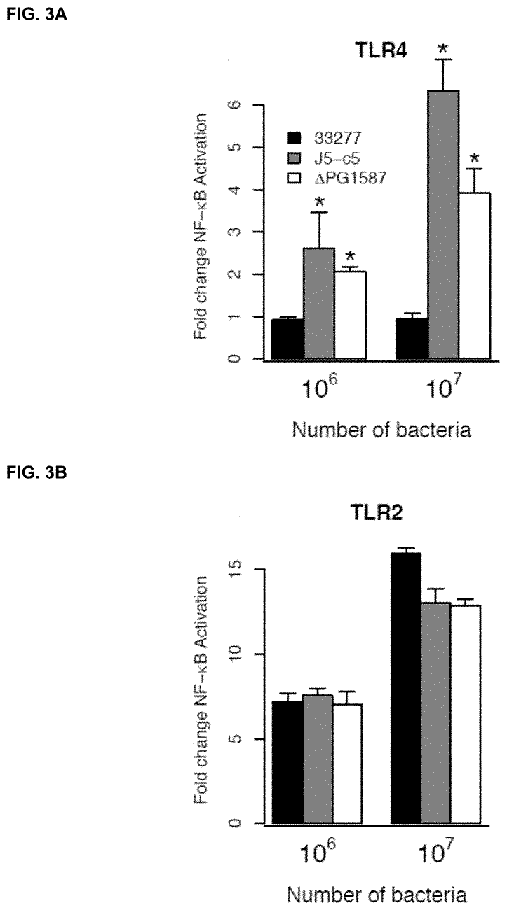

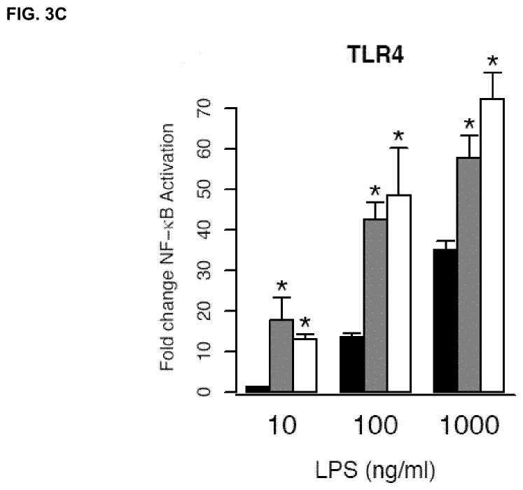

To determine whether the pro-inflammatory response triggered by the J5-c5 Tn mutant was mediated by activation of either TLR2 or TLR4 innate immune receptors, a HEK293 luciferase reporter assay (44) was utilized. Non-immune HEK293 cells were transfected with either TLR2 or TLR4-expressing plasmids, followed by infection with wild-type 33277, J5-c5 or ΔPG1587 intact bacterial cells. As seen in FIG. 3A, J5-c5 stimulated TLR4 strongly relative to 33277, similar to ΔPG1587 mutants, but did not increase levels of TLR2 stimulation (FIG. 3B). Since this assay measures fold change in activation, TLR4 activation by J5-c5 is highly significant.

The ability to stimulate TLR4 suggested lipid A structure in J5-c5 is different from that of wild-type, which was confirmed by subjecting purified LPS to a TLR4 assay. LPS from J5-c5, similar to LPS from ΔPG1587 mutants, stimulated TLR4 strongly relative to WT 33277 LPS (FIG. 3C), suggesting J5-c5 lipid A acylation status is shifted to a more penta-acylated profile. The possibility that the PG1587 gene, whose C4′-phosphatase activity was shown by us to be required for lipid A deacylation, is affected in J5-c5 by assaying for polymixin B sensitivity was ruled out. A characteristic feature of ΔPG1587 mutants is sensitivity to the cationic antimicrobial peptide polymixin B, on account of its lipid A being C4′-phosphorylated (29). J5-c5 mutants, however, displayed polymixin B resistance, similar to the wild-type bacterium, indicating the PG1587 gene, and the C4′-phosphatase activity it encodes, is intact in J5-c5.

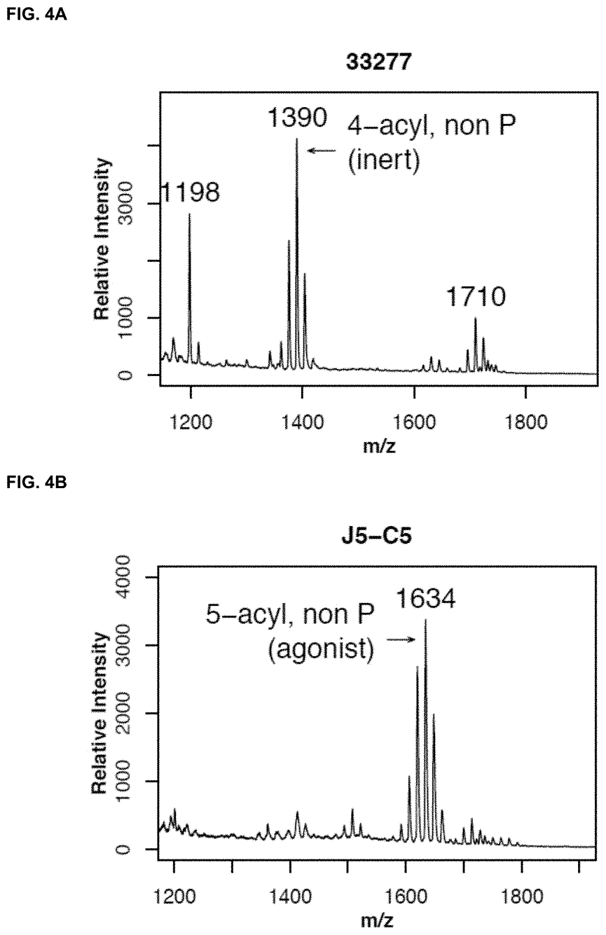

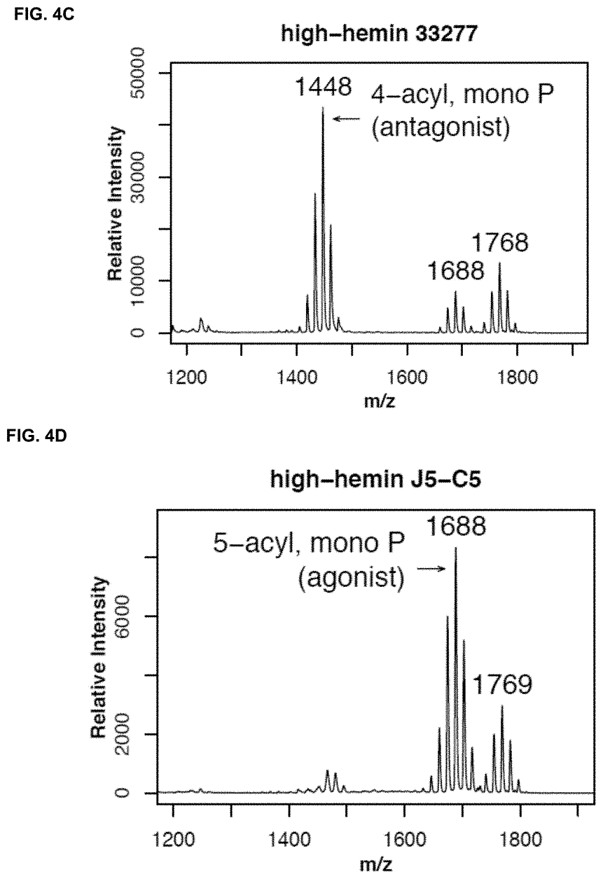

It was determined that the J5-c5 mutant lipid A structure by MALDI-TOF mass spectrometry (MS). As seen in FIG. 4B, lipid A of the J5-c5 mutant displayed predominantly penta-acylated non-phosphorylated lipid A when viewed in positive ion mode. This profile was strikingly different from that of wild-type, which displayed predominantly tetra-acylated non-phosphorylated lipid A (FIG. 4A). Tetra-acylated lipid A was abrogated to an undetectable level in J5-c5, indicating deacylase function is absent, or severely compromised.

Lipid A structure was also examined after growth of WT 33277 and J5-c5 mutant in high hemin conditions, a growth condition that abrogates C1-phosphatase activity (30). In WT, growth in high hemin leads to significant accumulation of antagonistic tetra-acylated C1-phosphorylated lipid A, as can be viewed in negative ion mode MALDI-TOF MS (FIG. 4C). This structure, however, was not detected in the largely penta-acylated J5-c5 lipid A profile (FIG. 4D), further confirming that deacylase function is severely compromised in this mutant.

Interestingly, non-, mono- and bis-phosphorylated forms of penta-acylated lipid A was detected in the J5-c5 mutant (FIGS. 4B and 4D). The non-phosphorylated aspect accounts for resistance of J5-c5 to polymixin B, while the penta-acylated feature accounts for high TLR4 stimulation. Non-phosphorylated penta-acylated lipid A is rarely observed in P. gingivalis 33277, but its production in J5-c5 indicates both C1- and C4′-phosphatases are active, further validating that PG1587 C4′-phosphatase has not been affected in J5-c5, hence indicating disruption of a novel gene.

The J5-c5 Mutant Contains Multiple Transposon Insertions.

Semi-random and nested PCR was used to identify the location of the transposon (Tn) insertion in J5-c5. An insertion was found to be located in an intergenic location, 122 bp downstream of PGN_0782 and 609 bp upstream of PGN_0783. Transposons, however, are capable of jumping to new locations until the replication-incompetent plasmid, which encodes both transposon and transposase, is lost from the population. A precise swap of the transposon with tetQ, a tetracycline resistance cassette, did not eliminate erythromycin resistance, indicating presence of additional transposons in J5-c5, which was confirmed by PCR (data not shown).

The entire J5-c5 genome was sequenced on Illumina's MiSeq platform to determine the number and location of transposons in J5-c5. Analysis of the J5-c5 sequencing reads revealed approximately 5× coverage of Tn sequence relative to the 33277 RefSeq chromosomal sequence, suggesting presence of five transposons in J5-c5. The insertion site of one of them, Tn-1, precisely matched the location identified by PCR-based methods described above. Location of four other transposons are listed in Table 2.

TABLE 2
Location of the five transposon insertions in J5-c5.

| | Chromosomal | |
|---|---|---|
| Name | position in 33277 | Genetic location |
| Tn-1 | 854,777 | Intergenic, 609 bp upstream |
| | | of PGN_0783, histone-like |
| | | DNA binding protein |
| Tn-2 | 62,040 | Intergenic, 52 bp upstream |
| | | of PGN_0053, hypothetical |
| | | protein, 408 aa |
| Tn-3 | 85,364 | In PGN_0081, matE family |
| | | efflux transporter |
| Tn-4 | 1,252,900 | In PGN_1124, hypothetical |
| | | protein, 326 aa |
| Tn-5 | 1,653,844 | Intergenic, 523 bp upstream |
| | | of PGN_1476, T9SS C-terminal |
| | | target domain-containing protein |

### Example 2

### Identification and Expression of the P. gingivalis Lipid A Deacylase Enzyme, PGN_1123

Materials and Methods

The studies conducted in working Example 2 include the materials and methods discussed in Example 1, as well as the following materials and methods.

Construction of the pJS46 expression vector. Generation of The PGN_1123 gene was amplified using primers 1123-start and 1123-stop (Table 1) by PCR. The 1.2 kb product was cloned into pGem-T-EZ by TA-cloning, generating pSJ831. It was next moved into a pT-COW-based plasmid called pSJ46, which is a broad host range vector capable of replicating in both E. coli and Bacteroidetes.

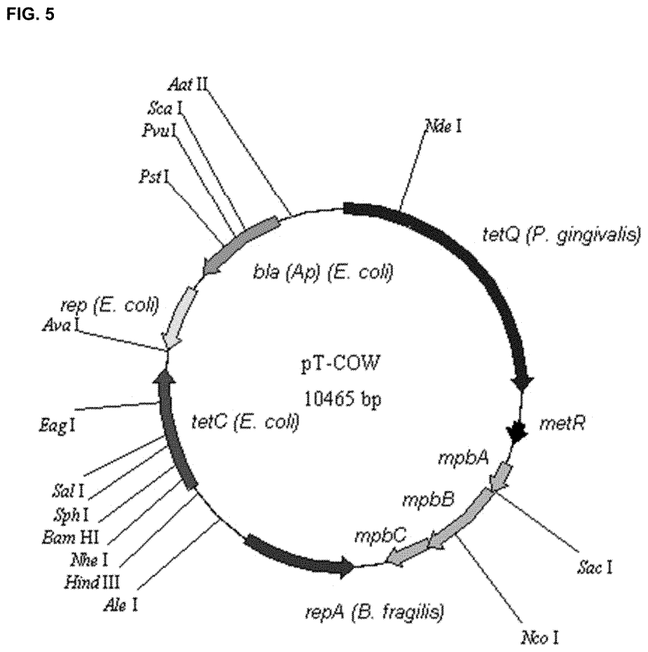

pSJ46 is an expression vector that was constructed for expressing genes in Porphyromonas gingivalis, Bacteroides species and potentially other bacteria belonging to the Order Bacteroidales. pSJ46 is derived from pT-COW. pT-COW is a plasmid vector that can replicate in P. gingivalis and Bacteroides as well as in E. coli, and is widely used in the P. gingivalis research field. A diagram of the pT-COW vector is shown in FIG. 5.

pT-COW encodes tetQ, conferring tetracycline resistance in Porphyromonas and Bacteroides. This vector was modified into the expression vector pSJ46 by removing the tetC gene, encoding tetracycline resistance in E. coli, introducing and replacing it with (1) a Bacteroides promoter for enabling transcription of Porphorymonas or Bacteroides genes (here, tetC was replaced with the promoter of the ermF gene, obtained from the ermFA erythromycin resistant cassette, and (2) a multiple cloning site (MCS) immediately downstream of the promoter for convenient insertion of the desired gene to be expressed.

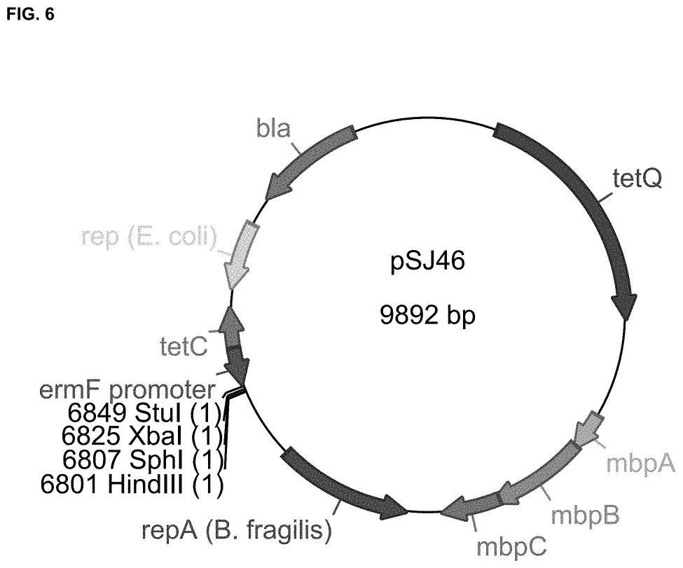

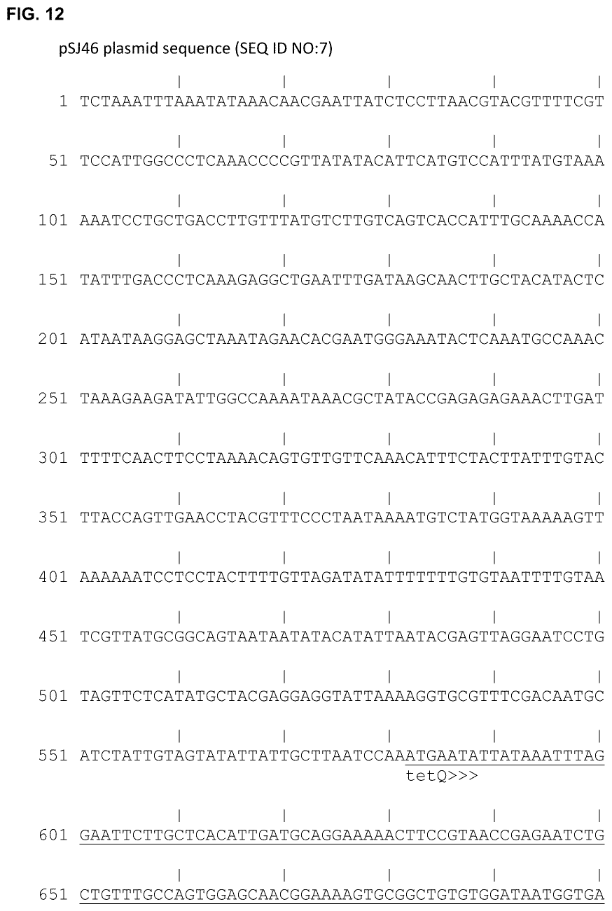

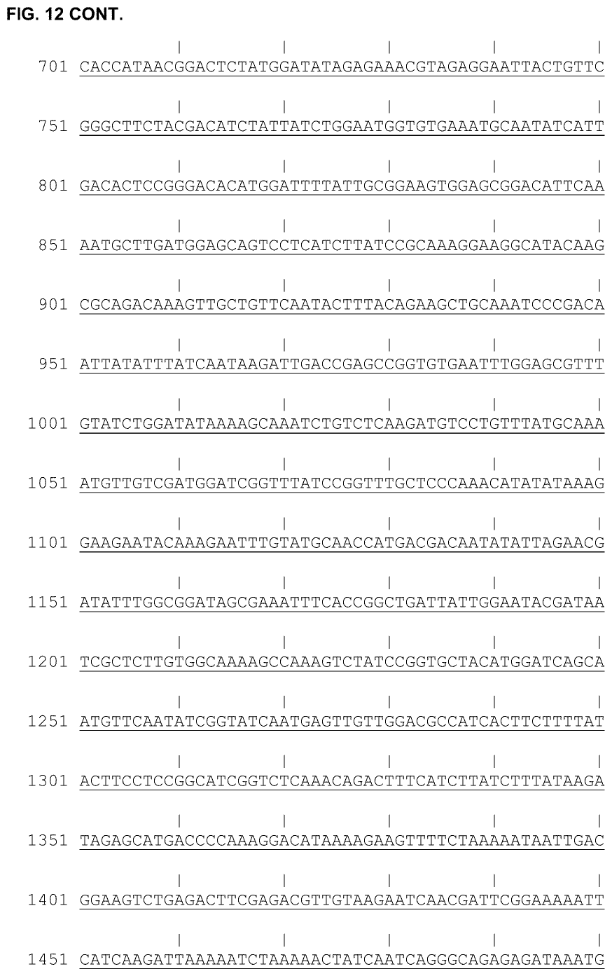

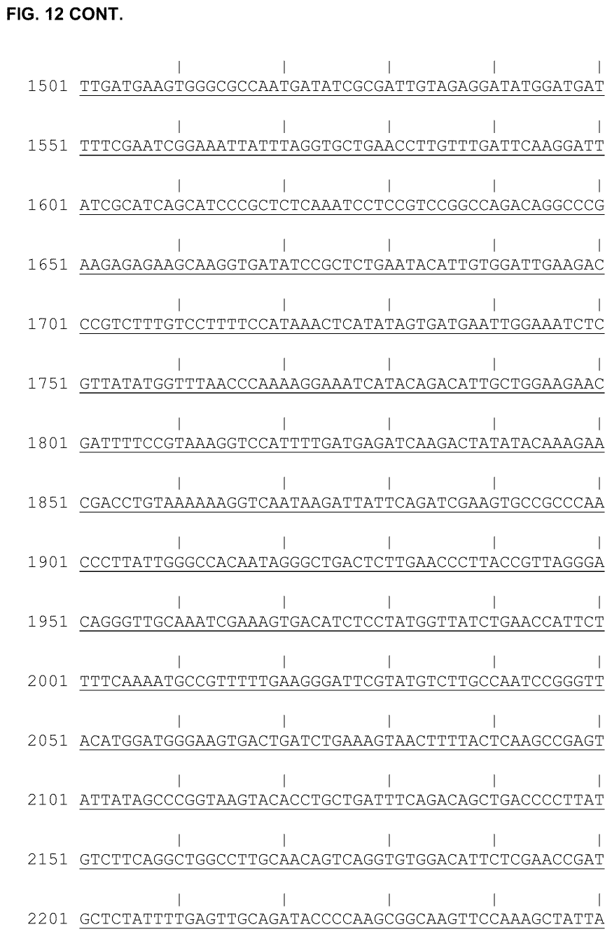

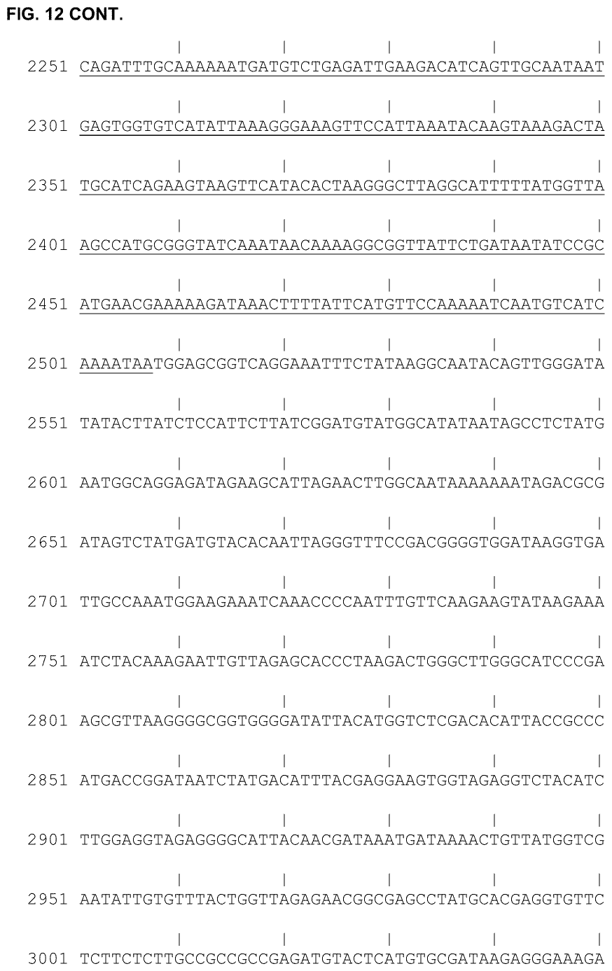

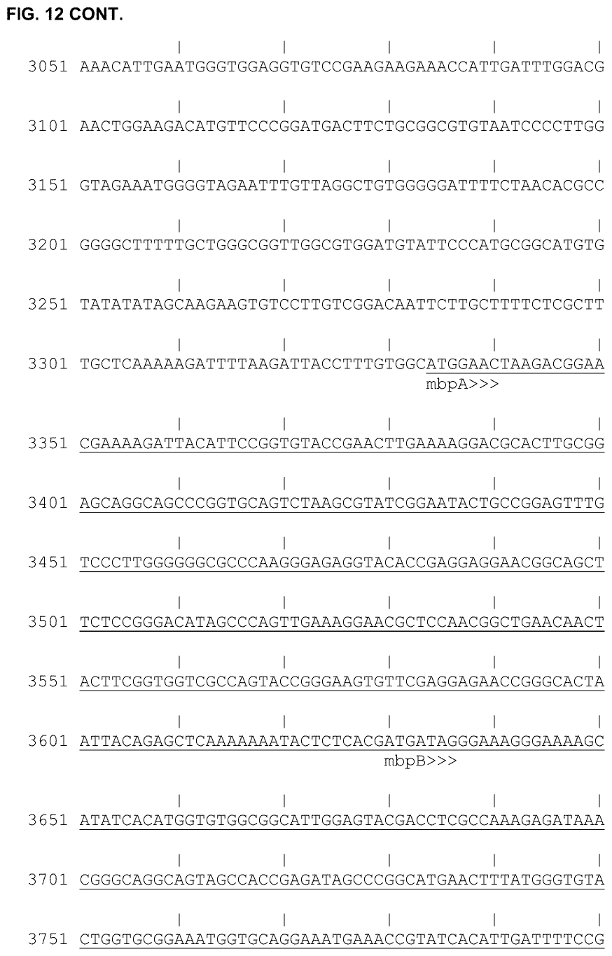

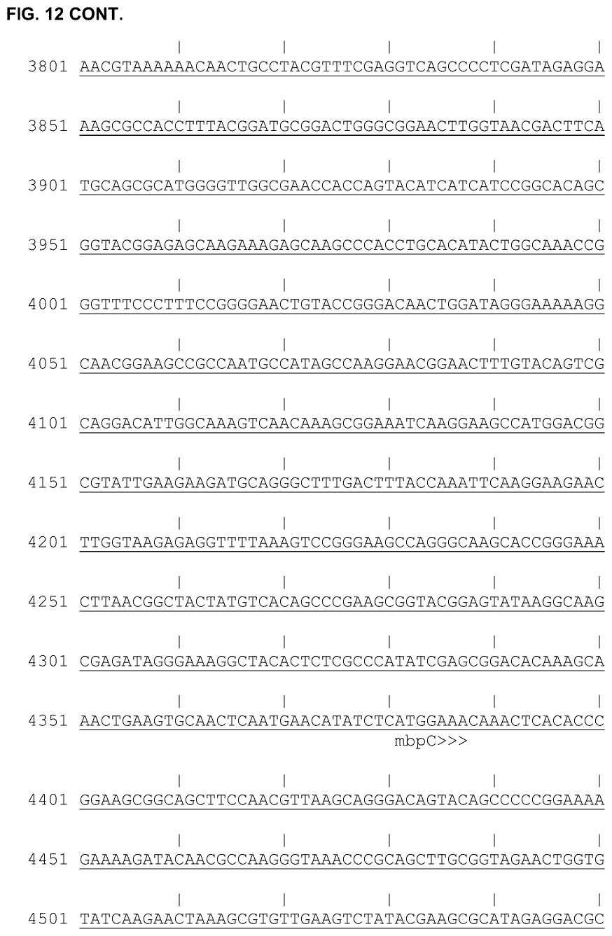

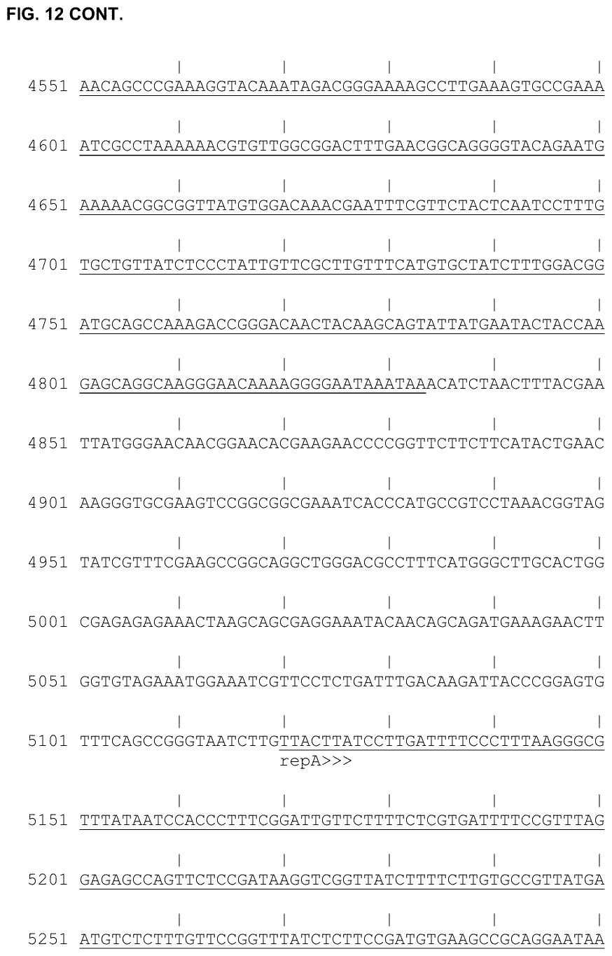

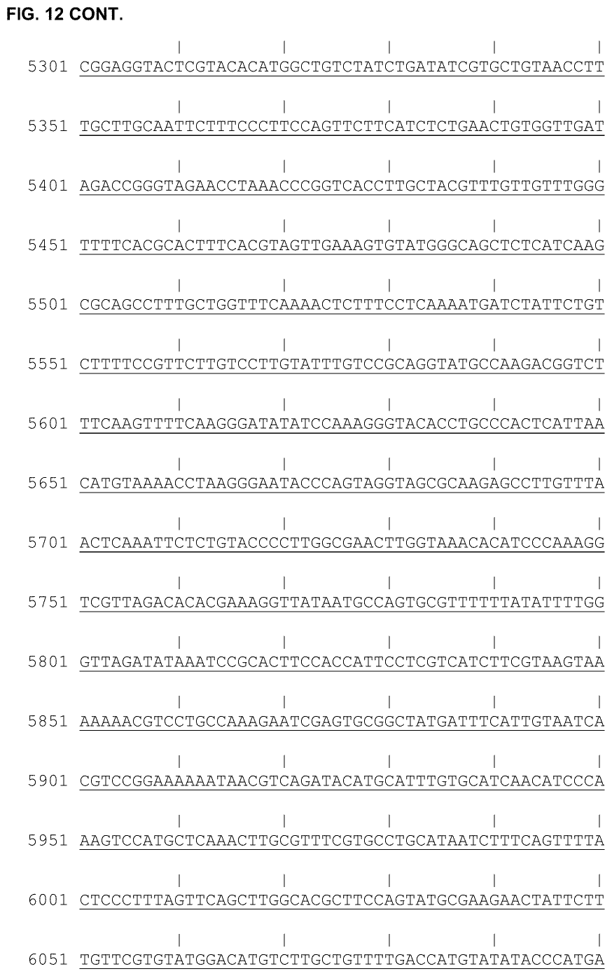

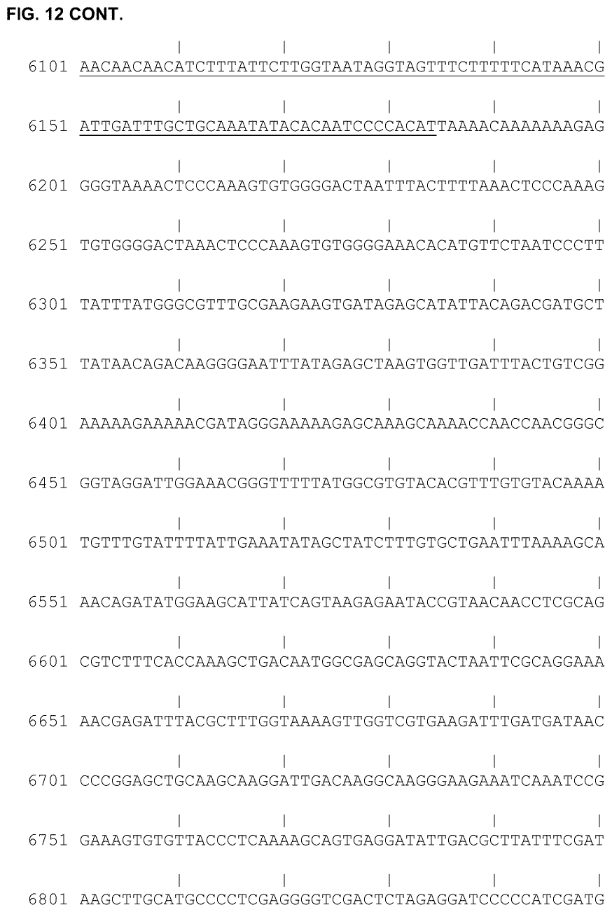

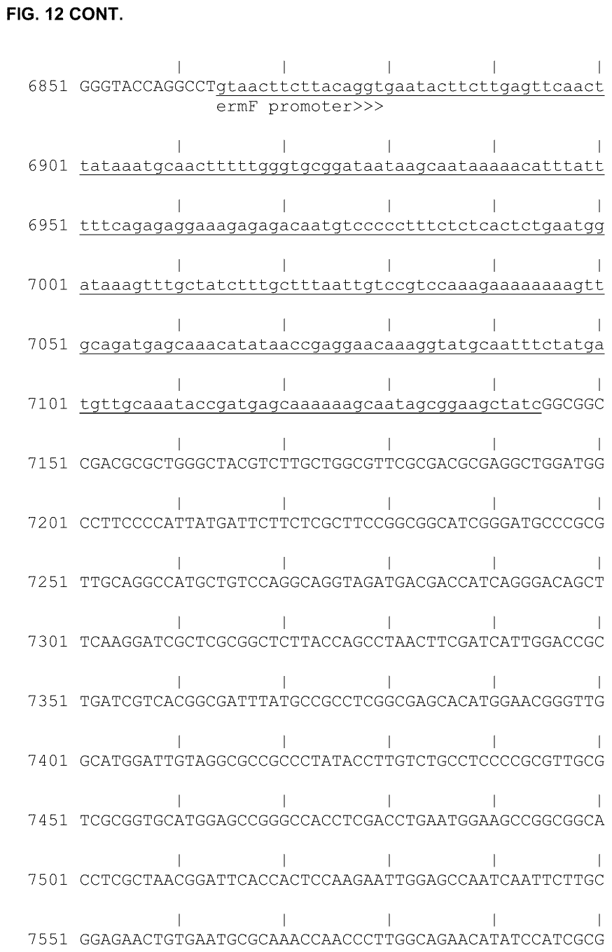

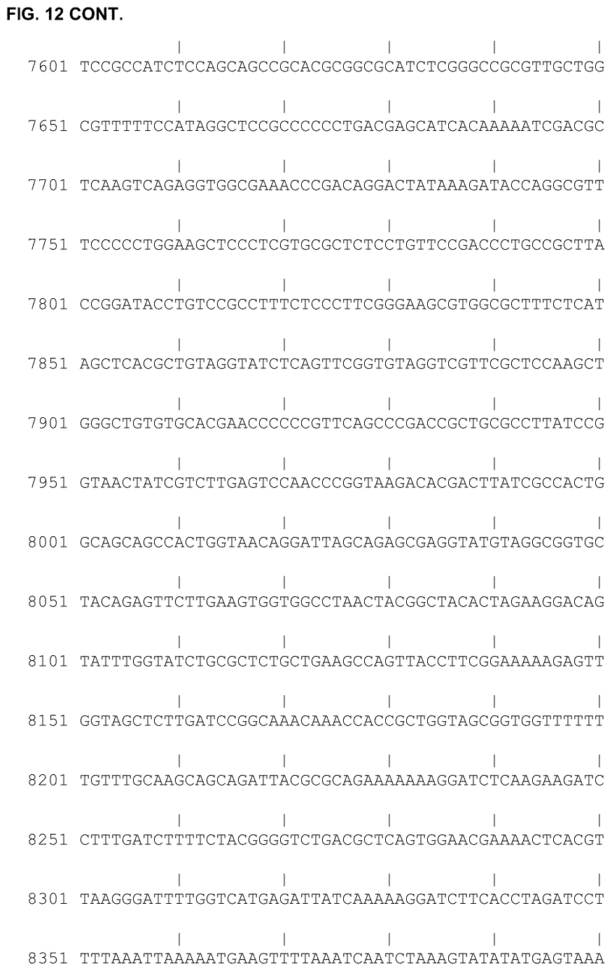

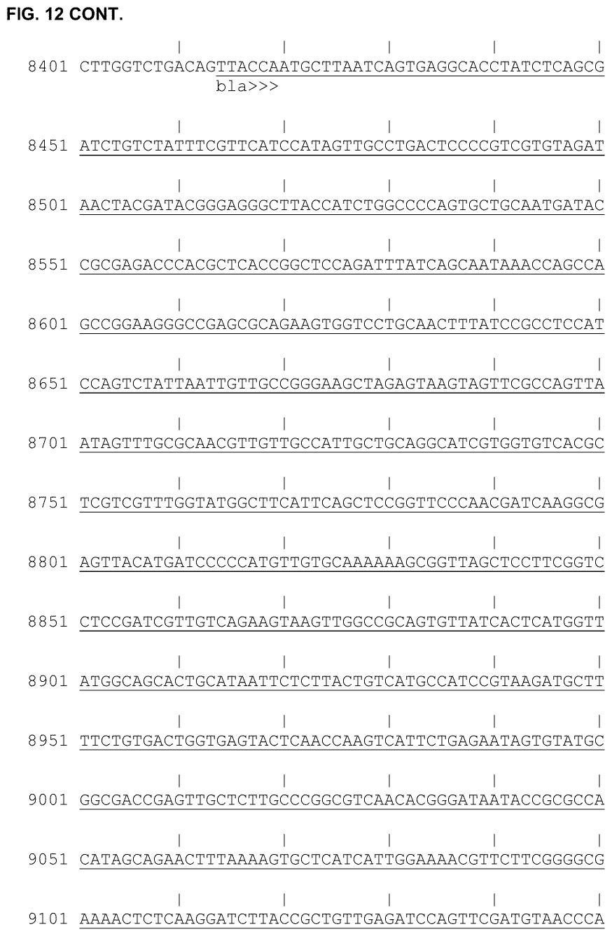

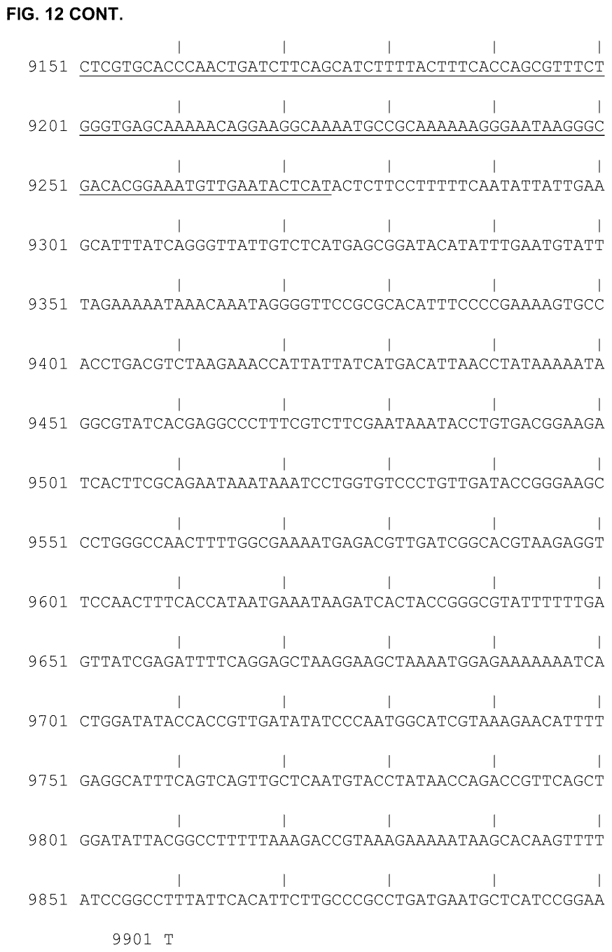

The unique restriction sites HindIII, just 5′ of tetC, and EagI, close to the 3′ end of tetC, were chosen and used for deletion of the tetC fragment. Next, a DNA fragment comprising 300 bp was added upstream of the ermF gene, which encodes for erythromycin resistance in Bacteroides, into the EagI-HinDIII sites along with an MCS. This 300 bp fragment was presumed to contain the promoter of the ermF gene. In this example, the majority of the tetC gene was removed by digesting pT-COW with EagI-HinDIII and replacing it with the ermF promoter+MCS. The ermF promoter was first cloned into the E. coli vector, pSU20 (which has an extensive MCS) using primers SJ59-SJ60 (Table 1) on an EcoRI-KpnI fragment. The promoter+MCS was subsequently sub-cloned into pT-COW on an EagI-HinDIII fragment to the EagI-HinDIII sites of the tetC-deficient pT-COW vector. The newly introduced EagI-HinDIII fragment contained both the ermF promoter and the multiple cloning site of pSU20. The resulting pSJ46 plasmid is shown in FIG. 6, and the pSJ46 plasmid sequence (SEQ ID NO:7) is shown in FIG. 12. Between the ermF promoter and the HindIII site is the newly introduced MCS comprising nine unique restriction sites, including StuI, XbaI and SphI (FIG. 6).

Construction of the pSJ836 plasmid. The plasmid, pSJ836, has the PGN_1123 gene from P. gingivalis strain 33277 cloned into pSJ46. PGN_1123 encodes the lipid A deacylase gene.

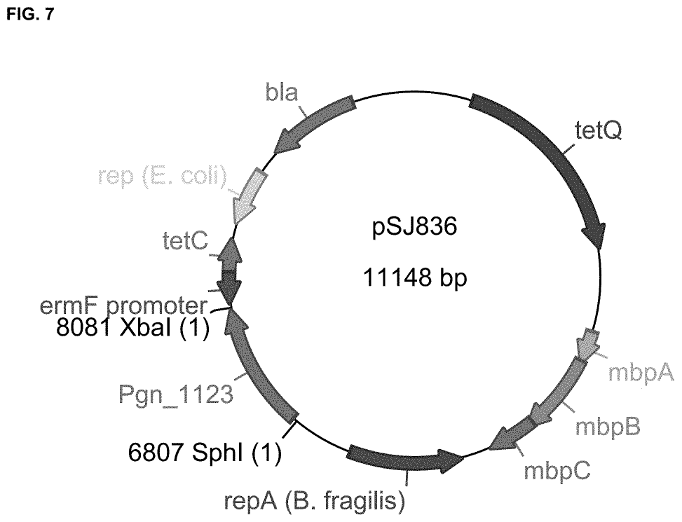

It was first attempted to clone the gene under the ermF promoter in pSJ46, into the StuI-XbaI sites, but at first, PGN_1123 was not able to be introduced under the ermF promoter in pSJ46. Next cloning was attempted in the orientation opposite (i.e., in the reverse orientation) to the ermF promoter, into the SphI-XbaI sites by digesting PGN_1123 out of pSJ831 on a SphI-XbaI fragment, and introducing into the same sites of pSJ46, generating the plasmid pSJ836. That strategy was successful, obtaining plasmid pSJ836, shown in FIG. 7. The pSJ836 plasmid sequence (SEQ ID NO:5) is shown in FIG. 13.

Generation of Porphyromonas gingivalis Mutant (pSJ836) (“PqΔ (pSJ836)”) and Bacteroides thetaiotaomicron (pSJ836) (“Bt (pSJ836)”) for Expression of PGN_1123 in Trans in P. gingivalis and B. thetaiotaomicron. The PGN_1123-containing pSJ836 plasmid was moved into P. gingivalis 33277 ΔPGN_1123:ermF mutants by conjugation to produce PgΔ (pSJ836). Tri-parental mating was used to mobilize plasmids from E. coli to P. gingivalis ΔPGN_1123, or to B. thetaiotaomicron (discussed below), with the help of the broad host range conjugative helper plasmid, pRK231.

Briefly, E. coli donor and helper strains were sub-cultured with no antibiotics and grown to an OD600 of ˜0.5. The recipient ΔPGN_1123 strain was grown to stationary phase, to OD600>2.0. 50 mls of recipient was spun and re-suspended in 5 mls TYK media, of which 750 μl was mixed with 250 μl donor and 250 μl helper E. coli strains. The mixture was spun down, pellet brought up in 100 μl TYK broth, and spotted on a TYHK agar plate. After aerobic incubation overnight at 37° C., the spotted mixture was transferred by swab to 1 ml TYK broth and mixed well by vortexing, 100 μl of the re-suspension was plated on TYHK media containing 0.5 μg/ml tetracycline, and incubated anaerobically. Tetracycline resistant ex-conjugants were obtained 4-5 days later. It was confirmed that PGN_1123 was expressed from pSJ836 in ΔPGN_1123::ermF mutants by RT-PCR, using purified RNA. This strain, ΔPGN_1123:ermF (pSJ836), successfully complemented the ΔPGN_1123 lipid A deacylase phenotype.

Expression of PGN_1123 from pSJ836 suggests presence of a mild promoter reading into the HindIII site (into the erstwhile tetC gene) that is capable of transcribing P. gingivalis genes. The MCS, in this scenario, is flanked by two promoters, one of which is strong (the ermF promoter) and the other is weak. The latter promoter may be conducive for cloning genes that are toxic in E. coli, of which PGN_1123 is a likely candidate.

The lipid A deacylase-containing plasmid, pSJ836, was similarly transferred by conjugation by tri-parental mating to B. thetaiotaomicron (Bt) to produce Bt (pSJ836). The method was similar to that with P. gingivalis except B. thetaiotaomicron was grown to an OD600 of ˜0.2, 700 μl of which was mixed with 150 μl E. coli donor and helper strains, also grown to OD600 of ˜0.2. Bt is a strain closely related to P. gingivalis (Pg); they both belong to the Order Bacteroidales. The lipid A structure of Bt is very similar to that of Pg. However, unlike Pg, the penta-acylated lipid A molecule in Bt, a strong TLR4 activator, is not modified. In Pg, the penta-acylated bis-phosphorylated structure is modified, notably by the newly identified lipid A deacylase, into tetra-acylated structures, which either suppresses TLR4 activation or is an inert TLR4 agonist. Hence, using this mechanism, P. gingivalis is capable of evading the powerful TLR4 response, unlike Bt.

When pSJ836 was introduced in trans in Bt, the Bt lipid A was also modified to give a tetra-acylated structure. The modified lipid A was a poor activator of TLR4. P. gingivalis lipid A deacylase, the product of the PGN_1123 gene, was therefore capable of deacylating Bt lipid A and quenching TLR4 stimulation.

PGN_1123 Encodes the Lipid A Deacylase

Since it was not clear which one or combination of Tn insertions was responsible for the deacylase phenotype, or, if indeed, whether the phenotype was due to direct interruption of a deacylase gene, or due to an indirect effect via gene regulation, RNAseq was employed to determine the genes that are differentially expressed between wild-type 33277 and the J5-c5 mutant. The top twenty genes displaying the most significant changes in log2 expression values in J5-c5 mutant relative to wild-type, as determined by utilizing edgeR with the default parameters described by Law et al (45), are listed in Table 3.

TABLE 3
Top 20 most significant changes in gene expression between wild-
type and J5-c5 transposon mutant as revealed by EdgeR analysis.

| | | Iog2 fold | |
|---|---|---|---|
| Name | Product | change | P. value |
| PGN_1123 | hypothetical_protein | −4.71590586 | 3.66E−06 |
| PGN_1281 | conjugative_transposon_protein_TraM | −2.276704451 | 0.000305862 |
| PGN_0783 | DNA-binding_protein | −7.281703828 | 0.00035261 |
| PGN_0090 | DNA-binding_protein | −2.304121237 | 0.000717639 |
| PGN_0066 | transposase | −2.142286175 | 0.000941805 |
| PGN_0060 | conjugative_transposon_protein_TraM | −2.198185031 | 0.000882388 |
| PGN_1124 | paraslipin | −1.690660121 | 0.001456315 |
| PGN_0084 | DNA_topoisomerase_I | −1.699090077 | 0.001497081 |
| PGN_0083 | hypothetical_protein | −1.703911427 | 0.001862623 |
| PGN_0065 | transposase | −1.954910211 | 0.001886738 |
| PGN_1283 | conjugal_transfer_protein_TraO | −1.856001435 | 0.002120805 |
| PGN_0784 | hypothetical_protein | −5.588007472 | 0.002152355 |
| PGN_0061 | transposase | −2.4521437 | 0.002448023 |
| PGN_0064 | transposase | −1.800519514 | 0.002979058 |
| PGN_1282 | conjugative_transposon_protein_TraN | −1.636844785 | 0.003527579 |
| PGN_0063 | conjugative_transposon_protein_TraJ | −1.868293938 | 0.004728484 |
| PGN_0194 | hypothetical_protein | 3.227338786 | 0.004526265 |
| PGN_0074 | hypothetical_protein | −2.247462691 | 0.007013359 |
| PGN_0901 | 6,7-dimethyl-8-ribityllumazine_synthase | 2.462105689 | 0.00738567 |
| PGN_0067 | transposase | −1.732652686 | 0.008098611 |

The statistical parameters of this package identified exactly one gene, PGN_1123, as significantly differentially expressed. PGN_1123 is located immediately downstream of the gene interrupted by Tn-4, PGN_1124. Besides PGN_1123, the only genes that displayed >4× fold decrease in log2 expression values were located on one side of Tn-1, PGN_0783 and PGN_784.

PGN_1123, PGN_1124 and PGN_0783 genes were targeted for deletion analysis in P. gingivalis 33277. Deletion mutations were constructed by replacing each gene with a non-polar erythromycin resistance cassette, ermF, using homologous recombination.

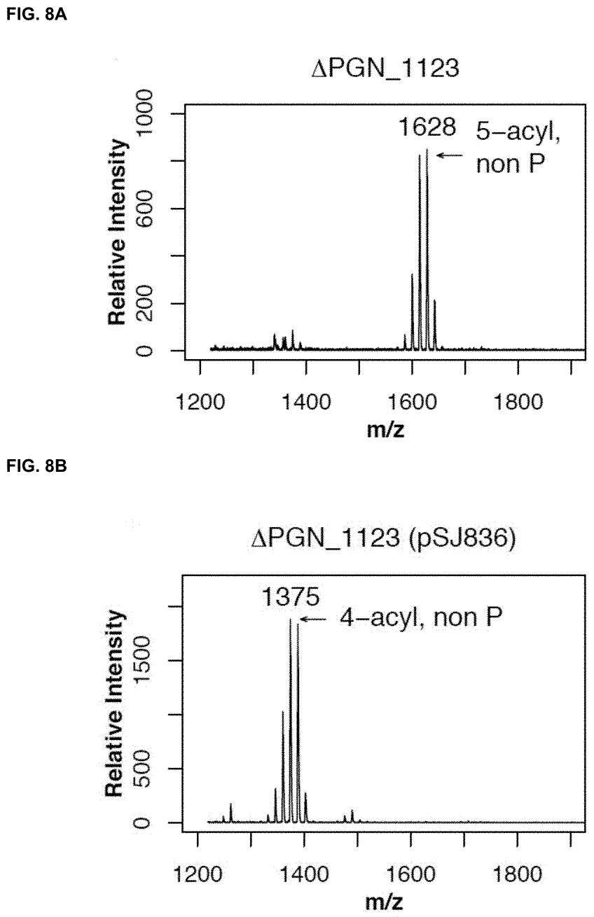

The deletion mutation in PGN_1123 was found to confer the lipid A deacylase phenotype, as demonstrated by structural and functional analyses. Lipid A of the ΔPGN_1123 mutant was devoid of tetra-acylated structures, and displayed predominantly penta-acylated non-phosphorylated lipid A (FIG. 8A), similar to the J5-c5 Tn mutant, as determined by MALDI-TOF MS. On the other hand, both ΔPGN_1124::ermF and ΔPGN_0783::emF mutants displayed the wild-type tetra-acylated phenotype as was seen in FIG. 4A (data not shown).

The ΔPGN_1123:ermF deacylase phenotype was successfully complemented by introducing a plasmid expressing PGN_1123 in trans. The PGN_1123 gene was cloned into pSJ46, a pT-COW-based plasmid vector capable of replicating in both E. coli and P. gingivalis, generating the plasmid pSJ836. Conjugation was used to mobilize pSJ836 from E. coli to ΔPGN_1123:ermF mutants, and expression of PGN_1123 gene confirmed by RT-PCR (data not shown). Analysis of ΔPGN_1123:ermF (pSJ836) lipid A demonstrated presence of tetra-acylated non-phosphorylated structures, similar to wild-type (FIG. 8B). Complementation of the ΔPGN_1123:ermF penta-acylated lipid A phenotype to tetra-acylated lipid A in ΔPGN_1123 (pSJ836) confirmed PGN_1123 is required for lipid A deacylation.

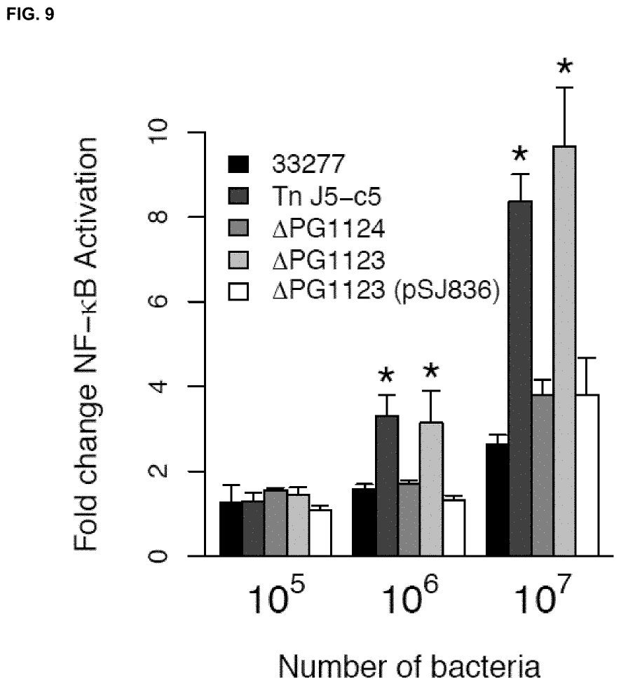

Consistent with an absence of tetra-acylated lipid A, infection of TLR4-expressing HEK293 cells with ΔPGN_1123:ermF mutant whole cells incurred potent TLR4 stimulation (FIG. 9), similar to J5-c5. ΔPGN_1124 mutants, on the other hand, displayed a non-activating phenotype similar to wild-type, indicating the gene interrupted by the Tn responsible for the deacylase phenotype due to polar effects on PGN_1123 itself does not play a role in TLR4 evasion. Introduction of pSJ836, the plasmid expressing PGN_1123, into ΔPGN_1123 mutants resulted in substantial reduction in TLR4 stimulation, confirming complementation of the lipid A deacylase phenotype.

Furthermore, replacement of Tn-4 alone, out of the five Tns present in J5-c5, with a nonpolar tetracycline resistance cassette also restored lipid A deacylase activity (data not shown), confirming Tn-4 was the sole Tn in J5-c5 responsible for conferring the lipid A deacylase phenotype, specifically by exerting polar effects on downstream gene transcription.

PGN_1123 Encodes a Novel Phylogenetically Restricted Deacylase, and is Part of an Operon

PGN_1123 is annotated to encode a conserved hypothetical protein comprising 399 amino acid residues. Homology searches did not reveal any obvious similarity to a protein of known function, and the protein does not display known motifs. PGN_1123 is highly conserved in P. gingivalis, with homologs displaying 92-98% identity present in all sequenced P. gingivalis strains. An ortholog with 92% identity was found in Porphyromonas gulae, after which identity to the next closest homolog fell to 43%, in P. crevioricanis. Orthologs with 30-35% identity were found in several bacteria, all belonging to the Bacteroidetes phylum, including Prevotella stercorea (35% identity), Tannerella forsythia (36% identity), Parabacteroides goldsteinii (35% identity) and Bacteroides fragilis (33% identity). Comparison of PGN_1123 protein with the known deacylases, PagL and IpxR, revealed low identity, <15%. Since PagL and IpxR are not orthologs of each other either and exhibit differences in their modes of action, PGN_1123 represents a third distinct class of bacterial lipid A deacylase enzymes.

Both PagL and IpxR have been shown to be outer membrane proteins with β-barrel structures (46, 47). Using several web-based tools, PGN_1123 is predicted to have a signal sequence, required for secretion across the inner membrane in Gram-negative bacteria, with a potential cleavage site located after the 19th amino acid. One of several tertiary structure predictions of PGN_1123 using the I-TASSER server at the Univ. of Michigan (48) revealed a β-barrel structure.

The start codon of the PGN_1123 gene overlaps with the stop codon of the gene upstream, PGN_1124, suggesting the two genes are translationally coupled. Indeed, it has been demonstrated that the homologs in P. gingivalis W83, PG1334 and PG1333, are co-transcribed together with the gene upstream, PG1335 (49), indicating the three genes are organized in an operon. The first gene of the operon, PG1335 in W83, is 27 bp upstream of the second gene, PG1334. The three genes, PGN_1125-1124-1123 318 (PG1335-1334-1333 in W83), are all conserved in sequenced P. gingivalis strains.

PG1334, the middle gene of the operon in W83, interestingly, was identified in an IVIAT (in vivo induced antigen technology) study as a gene that is induced in vivo during human periodontitis (50). Subsequent qRT-PCR studies by Walters et al confirmed the PG1334 transcript is detected more frequently in dental plaque samples from periodontal diseased sites than from healthy sites, after taking into account P. gingivalis abundance, consistent with induction of expression during disease (49).

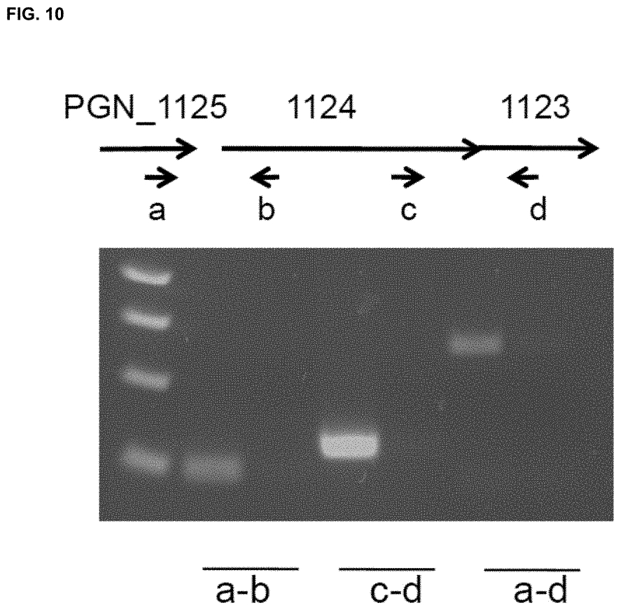

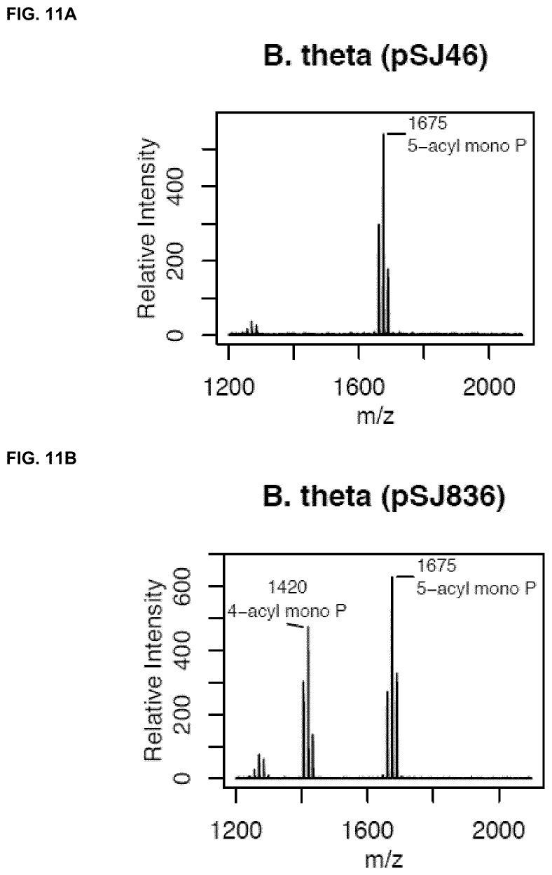

To confirm the three genes are co-transcribed in 33277 as well, semi-quantitative RT-PCR was performed, as shown in FIG. 10. It was demonstrated that the three genes form an operon based on the detection of mRNA overlapping PGN_1125-1124 genes, PGN_1124-1123, and, importantly, PGN_1125-1123, the first and last genes. This strongly suggests PGN_1123 is induced in vivo during disease.

PGN_1123 Deacylates Bacteroides thetaiotaomicron Lipid A

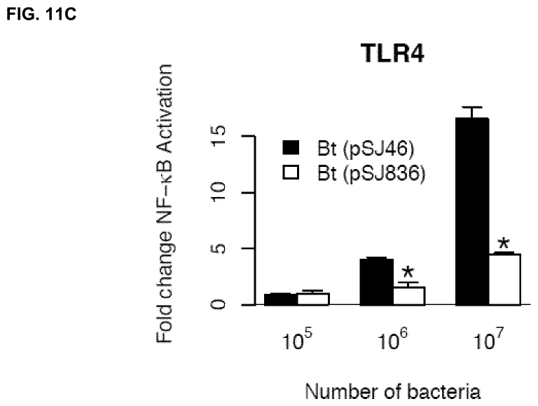

In order to obtain definitive evidence that PGN_1123 is a structural gene encoding the lipid A deacylase, PGN_1123 was heterologously expressed in B. thetaiotaomicron, a close phylogenetic relative of P. gingivalis, both belonging to the Order Bacteroidales in Phylum Bacteroidetes. Lipid A from B. thetaiotaomicron is penta-acylated C1-phosphorylated (FIG. 11A, m/z 1675), structurally similar to the penta-acylated C4′-phosphorylated lipid A cluster seen in P. gingivalis. Tetra-acylated lipid A was not known to have been observed before in B. thetaiotaomicron, nor was the bacterium contain a PGN_1123 homolog when searched by BLAST tools, which is in marked contrast to B. fragilis. B. thetaiotaomicron LPS is a strong activator of TLR4, stronger than P. gingivalis LPS, due to location of the phosphate group at the C1 position instead of the C4′ position (51). Strong TLR4 activation by B. thetaiotaomicron LPS, and by whole bacteria, suggests that, unlike in P. gingivalis, there are no attenuating tetra-acylated lipid A structures capable of dampening TLR4 activation.

Consistent with PGN_1123 encoding deacylase activity, B. thetaiotaomicron harboring pSJ836 in trans led to deacylation of penta-acylated mono-phosphorylated lipid A to tetra-acylated mono-phosphorylated lipid A (m/z 1420), as revealed by MALDI-TOF MS in the negative ion mode (FIG. 11B). This marks the first time tetra-acylated lipid A has been observed in B. thetaiotaomicron. A HEK TLR4 assay revealed that TLR4 activation by B. thetaiotaomicron (pSJ836) intact bacteria was substantially lower than B. thetaiotaomicron containing vector alone (FIG. 11C), providing novel evidence that expression of the PGN_1123 gene product is sufficient to confer a TLR4 evasive phenotype in B. thetaiotaomicron. Lipid A deacylation in B. thetaiotaomicron by heterologous expression of PGN_1123 confirms PGN_1123 encodes a lipid A deacylase.

Discussion

Identification of PGN_1123 as the lipid A deacylase encoding gene in P. gingivalis is reported herein, a gene that plays a pivotal role in evasion and suppression of the TLR4-mediated host innate immune response. Identification of this gene has been sought since TLR4 antagonism by tetra-acylated lipid A in P. gingivalis was first described, over two decades ago. The inability to identify this gene by homology searches is now clear since PGN_1123 does not display homology to the two known deacylases, PagL and IpxR. Additionally, the sequence of PGN_1123 is unique in that it does not exhibit close homologs outside of P. gingivalis (with the exception of P. gulae) nor does it contain motifs that could inform us on its function. In this report, it was shown PGN_1123 is required for lipid A deacylation by structural and functional studies, and established that it encodes the lipid A deacylase enzyme by demonstrating deacylation following heterologous expression in B. thetaiotaomicron.

Lipid A is a fundamental component of the bacterial outer membrane. Re-modeling the membrane by lipid A deacylation has been shown to play a significant role in pathogenesis in several bacteria on account of its moderating effect of the immune response. Lipid A deacylation in B. thetaiotaomicron as mediated by PGN_1123 did, indeed, attenuate TLR4 activation, leading us to speculate on the reason for absence of the genetic capability for deacylation in this intestinal commensal bacterium, resulting in synthesis of a lipid A structure that is an unequivocal stimulator of TLR4. B. fragilis, belonging to the same genus and considered an opportunistic pathogen, on the other hand, possesses an ortholog of the PGN_1123 protein, suggesting it has the capacity to undergo lipid A deacylation. This bacterium, however, has not been shown to display deacylated lipid A when grown in vitro. Further studies are warranted to more fully understand the expression of putative deacylase genes in Bacteroides sp. Notably, PGN_1123 belongs to a third distinct class of bacterial lipid A deacylase enzymes and it remains feasible there are other novel classes that remain unidentified.

Many pathogens possess the ability of disrupting TLR4 signaling by lipid A deacylation. S. typhimurium, for example, undergoes pagL-dependent lipid A deacylation in response to low pH and low Ca++/Mg++ concentration as would be encountered in phagolysosomes, suggesting deacylation is important for pathogenesis (52). Pseudomonas aeruginosa lipid A was shown to undergo deacylation in isolates from cystic fibrosis patients by up-regulation of pagL expression, which was not seen in environmental isolates (53). A striking example of de-acylated lipid A contributing to pathogenesis is seen in Yersinia pestis, the etiologic agent of bubonic plague, which shuttles between a flea vector and humans. When incubated at 27° C., the environmental temperature of its flea vector, lipid A was composed of predominantly hexa-acylated lipid A structures, but when incubated at 37° C., the mammalian host temperature, a shift to a tetra-acylated form was observed (54, 55). The temperature dependent shift in lipid A structure was shown to occur in Y. enterocolitica and Y. pseudotuberculosis as well, and IpxR activity was demonstrated to remove the 3′-acyl-oxy-acyl chain in Y. enterocolitica (56). Tetra-acylated lipid A in Y. pestis, which is considered to be formed by decreased acyl transferase activity, rendered the bacterium silent to TLR4 recognition and reduced the inflammatory response, hence potentially allowing the pathogen to proliferate undetected in the bloodstream during early stages of infection. Interestingly, genetic modification of tetra-acylated lipid A to a hexa-acylated structure led to potent TLR4 stimulation but rendered the strain avirulent in a mouse model of infection (57), highlighting the important role of reduced number of acyl chains in pathogenesis.

P. gingivalis is a unique member of the sub-gingival plaque microbial community in that it has the distinct capacity to foil TLR4 signaling, even antagonizing it, giving it the capacity to protect itself and, potentially, by-stander bacteria in the plaque community as well. This is consistent with the observation that P. gingivalis is observed in high abundance in diseased plaque sites, and is also associated with an increase in microbial load and an alteration in plaque microbial composition in animal models of infection. P. gingivalis hence has pathogenic properties that can be significantly attributed to TLR4 evasion by tetra-acylated lipid A. The phenotype of P. gingivalis ΔPGN_1123 mutants in vivo with respect to colonization, instigation of dysbiosis, and chronic inflammation remains to be tested. Mutants displaying penta-acylated lipid A due to deletion of the PG1587 gene were shown to stimulate a pro-inflammatory response and displayed reduced survival in a mouse macrophage model of infection (33). ΔPG1587 mutants were shown to be defective for colonization in a rabbit model of infection as well (17), indicating lipid A deacylation is required for pathogenesis. The distinction, if any, between the penta-acylated lipid A mutants, ΔPGN_1123 and ΔPG1587, which possess largely non-phosphorylated lipid A vs. exclusively C4′-phosphorylated lipid A respectively, will become clear from future in vivo studies.

Expression of the PGN_1123 gene is predicted to be up-regulated during disease as inferred from up-regulation of the co-transcribed PGN_1124 gene during disease. Gene regulation has been reported for both pagL and IpxR deacylases, by several molecular mechanisms, and in response to environmental conditions. pagL transcription in S. typhimurium, for example, is activated by the two-component regulator, PhoPQ, in response to different host microenvironments that include acidic pH, cationic anti-microbial peptides, and depletion of Ca2+ and Mg2+ (34). Non-coding sRNA molecules are known to modulate expression of outer membrane proteins, and have been shown to regulate both pagL and IpxR. MicF is an sRNA that binds to IpxR transcripts in Salmonella, increasing degradation of the mRNA by exposing regions that are susceptible to RNase E (58). A recent study identified an sRNA, Sr006, as a positive regulator of pagL expression in P. aeruginosa, leading to reduction in the pro-inflammatory response (59). Another recent study in EHEC (enterohaemorrhagic E. coli) identified two virulence regulators, Ler and Pch, as positive regulators of IpxR (60).

Increased expression of PGN_1123 during disease would correlate with enhanced survival during inflammatory conditions. The co-transcribed PGN_1124, shown to be expressed prominently during disease, has a motif indicating it belongs to the Band 7 family of proteins, a family conserved from protozoa to mammals. Also called the slipin family (stomatin-like integral membrane protein), it is implicated with regulating membrane protein turnover in stomatins and other membrane-associated proteins. It is often co-transcribed with a gene encoding a membrane protease, which in P. gingivalis is predicted to be encoded by PGN_1125, the first gene of the operon. Genes in an operon often are part of the same functional pathway, but since a deletion mutation in PGN_1124 did not affect lipid A deacylase function (FIG. 9) or structure (data not shown), the reason for these three genes to be in an operon is not clear. Interestingly, transcript overlapping PGN_1124 and PGN_1123, the second and third genes of the operon, was more abundant than that overlapping the first two genes, or of all three genes (FIG. 10), indicating an additional level of transcriptional control for PGN_1123 expression independent of the operon.

In addition to TLR4 evasion, it is becoming established that lipid A deacylation contributes to pathogenesis in bacteria by facilitating the formation of outer membrane vesicles (OMVs) (61-63). Fewer acyl chains in the lipid A moiety leads to a reduction in the hydrophobic area of the molecule The associated change in membrane structure, from a cone to either a cylinder or inverted cone, has been proposed to facilitate increased membrane curvature, the first step towards outer membrane blebbing and OMV formation (64).

P. gingivalis is known to release a large number of OMVs, which may be a function of its largely tetra-acylated lipid A profile. OMVs from P. gingivalis mediate co-aggregation with other bacteria, heme acquisition, and, furthermore, are known to themselves invade host epithelial tissue with, as it turns out, a large arsenal of virulence proteins (62, 63). The proteomic and LPS content of P. gingivalis OMVs has been shown to be different from that of the outer membrane (OM) by having (a) elevated levels of proteins secreted by the Type IX secretion system (T9SS), which include gingipain proteases and other virulence factors, and (b) increased proportion of anionic A-LPS relative to neutral O-LPS (65, 66). Co-localization of Type IX secreted proteins and A-LPS is consistent with the finding that T9SS proteins use A-LPS as an anchor for attachment to the outer membrane (67-69). Interestingly, many bacteria have been shown to pack a preferential cargo of proteins into their OMVs (70-75), suggesting membrane re-modeling occurs prior to, and may facilitate, vesiculation.

Lipid A content of P. gingivalis OMVs was also shown to be different from that of the OM by being nearly devoid of penta-acylated lipid A structures, instead comprised by tetra- and/or tri-acylated lipid A, leading the authors to conclude lipid A in OMVs is more heavily deacylated than lipid A in OMs (32, 65). Interestingly, a recent study on an S. typhimurium strain expressing recombinant pagL deacylase demonstrated lipid A deacylation from hexa- to penta-acylated structures as expected, but which, notably, led to increased vesiculation (61). Moreover, the deacylated (penta-acyl) lipid A was detected exclusively in OMVs and not in the bacterial OM, suggesting PagL-mediated changes in membrane structure contributed to membrane curvature and to formation of OMVs.

It was recently reported in a collaborative study that non-deacylated (penta-acyl) lipid A from P. gingivalis ΔPG1587 mutants is not only an agonist for TLR4 stimulation but also an agonist of inflammasome activation (33), an immune pathway that targets intra-cellular cytosolic bacteria for elimination by pyroptosis. Bacterial lipid A has been shown to bind murine caspase-11, and its homolog in humans, caspase-4/5, resulting in inflammasome activation by the non-canonical pathway (76, 77). Under-acylated lipid A, on the other hand, specifically tetra-acylated forms, was shown to bind caspases but failed to induce activation. Hence, lipid A structural modifications that abrogate TLR4 activation also abrogate inflammasome activation, as has been shown by us with P. gingivalis and by others in bacteria such as Francisella novicida.

In summary, lipid A deacylation has been shown to be involved in a wide range of functions that each contribute to pathogenesis. Identification of PGN_1123 as the deacylase encoding gene in P. gingivalis will now enable us to gain further insights into the precise role of deacylation in immune evasion, virulence factor expression, and, furthermore, in the potential contribution in vivo towards survival, dysbiosis and chronic inflammation. Identification also lays the foundation for investigating the use of deacylase gene expression as a potential diagnostic tool, and inhibition of the enzyme as a potential therapeutic, for periodontitis.

### Example 3

### Generation of PSJ922 Plasmid and Bacteroides thetaiotaomicron (PSJ836) (“BT (PSJ922)”) for Expression of PGN_1123 in Trans in B. thetaiotaomicron

The PGN_1123 sequence contained in the pSJ836 plasmid discussed above was determined to have a point mutation. The PGN_1123 gene was again cloned into the same sites, SphI-XbaI, described above with respect to the pSJ46 plasmid. The PGN_1123 sequence in the resulting plasmid, referred to herein in pSJ922, showed no errors. pSJ922 complemented the 33277 ΔPGN_1123 deacylase mutant, and, when introduced into B. thetaiotaomicron to generate the Bt (pSJ922) strain, de-acylated its penta-acylated lipid A structure. FIG. 7 shows a schematic of the pSJ922 plasmid, and the pSJ836 plasmid sequence (SEQ ID NO:5) is shown in FIG. 14.

### Example 4

### Assay for Inhibitors of the Lipid a Deacylase using Bt (pSJ836) or Bt (pSJ922)

The studies described herein may be used to find an inhibitor of the P. gingivalis lipid A deacylase (PGN_1123 in 33277). Inhibition of the deacylase would lead to TLR4 stimulation by the resulting penta-acylated lipid A structure. A strong TLR4 response will presumably impede survival of P. gingivalis, a contributing factor towards development of periodontitis. P. gingivalis is considered to be key in instigating dysbiosis in the sub-gingival plaque community, tilting the environment to a chronic pro-inflammatory state. Eliminating P. gingivalis would likely eliminate a crucial risk factor for the development of periodontitis.

An assay for inhibition of lipid A deacylase activity in the Bacteroides strain Bt (pSJ836) or Bt (pSJ922) may be used to screen for molecules that restore TLR4 activity. For example, the assay may be an assay using a HEK luciferase or beta lactamase as a detector of activity. Those molecules that restore TLR4 activity will be identified candidate deacylase inhibitors. The assay will be robust in that compounds that inhibit growth of the bacteria by interfering with any other target will not give a robust TLR4 signal. Below depicts an example of assay results using this method:

| | TLR4 + (blue color) | TLR4 (−) clear |
|---|---|---|
| B. thetaiotaomicron (wt) | +++ | |
| B. thetaiotaomicron | | +++ |
| plus Pg deacylase* | | |
| B. thetaiotaomicron | | +++ |
| plus Pg deacylase* | | |
| no inhibitor | | |
| B. thetaiotaomicron | +++ | |
| plus Pg deacylase* | | |
| with inhibitor | | |

*B. thetaiotaomicron plus Pg deacylase refers to Bt (pSJ836) or Bt (pSJ922)

### REFERENCES

1. Darveau R P. 2010. Periodontitis: a polymicrobial disruption of host homeostasis. Nat Rev Microbiol 8:481-490.
2. Socransky S S, Haffajee A D. 1994. Evidence of bacterial etiology: a historical perspective. Periodontol 2000 5:7-25.
3. Rafiei M, Kiani F, Sayehmiri K, Sayehmiri F, Tavirani M, Dousti M, Sheikhi A. 2018. Prevalence of Anaerobic Bacteria (P. gingivalis) as Major Microbial Agent in the Incidence Periodontal Diseases by Meta-analysis. J Dent (Shiraz) 19:232-242.
4. Hajishengallis G, Lamont R J. 2014. Breaking bad: manipulation of the host response by Porphyromonas gingivalis. Eur J Immunol 44:328-338.
5. Zenobia C, Hajishengallis G. 2015. Porphyromonas gingivalis virulence factors involved in subversion of leukocytes and microbial dysbiosis. Virulence 6:236-243.
6. Darveau R P, Belton C M, Reife R A, Lamont R J. 1998. Local chemokine paralysis, a novel pathogenic mechanism for Porphyromonas gingivalis. Infect Immun 66:1660-5.
7. Hasegawa Y, Tribble G D, Baker H V, Mans J J, Handfield M, Lamont R J. 2008. Role of Porphyromonas gingivalis SerB in gingival epithelial cell cytoskeletal remodeling and cytokine production. Infect Immun 76:2420-2427.
8. Kadowaki T, Takii R, Yamatake K, Kawakubo T, Tsukuba T, Yamamoto K. 2007. A role for gingipains in cellular responses and bacterial survival in Porphyromonas gingivalis-infected cells. Front Biosci 12:4800-9.
9. Curtis M A, Aduse-Opoku J, Rangarajan M. 2001. Cysteine proteases of Porphyromonas gingivalis. Crit Rev Oral Biol Med 12:192-216.
10. Wang M, Shakhatreh M-A K, James D, Liang S, Nishiyama S-I, Yoshimura F, Demuth D R, Hajishengallis G. 2007. Fimbrial proteins of Porphyromonas gingivalis mediate in vivo virulence and exploit TLR2 and complement receptor 3 to persist in macrophages. J Immunol 179:2349-2358.
11. Hajishengallis G, Wang M, Liang S, Shakhatreh M A, James D, Nishiyama S, Yoshimura F, Demuth D R. 2008. Subversion of innate immunity by periodontopathic bacteria via exploitation of complement receptor-3. Adv Exp Med Biol 632:203-19.
12. Bielecka E, Scavenius C, Kantyka T, Jusko M, Mizgalska D, Szmigielski B, Potempa B, Enghild J J, Prossnitz E R, Blom A M, Potempa J. 2014. Peptidyl arginine deiminase from Porphyromonas gingivalis abolishes anaphylatoxin C5a activity. J Biol Chem 289:32481-32487.
13. Lasica A M, Ksiazek M, Madej M, Potempa J. 2017. The Type IX Secretion System (T9SS): Highlights and Recent Insights into Its Structure and Function. Front Cell Infect Microbiol 7.
14. Darveau R P, Hajishengallis G, Curtis M A. 2012. Porphyromonas gingivalis as a potential community activist for disease. J Dent Res 91:816-820.
15. Hajishengallis G, Darveau R P, Curtis M A. 2012. The keystone-pathogen hypothesis. Nat Rev Microbiol 10:717-725.
16. Hajishengallis G, Liang S, Payne M A, Hashim A, Jotwani R, Eskan M A, McIntosh M L, Alsam A, Kirkwood K L, Lambris J D, Darveau R P, Curtis M A. 2011. Low-abundance biofilm species orchestrates inflammatory periodontal disease through the commensal microbiota and complement. Cell Host Microbe 10:497-506.
17. Zenobia C, Hasturk H, Nguyen D, Van Dyke T E, Kantarci A, Darveau R P. 2014. Porphyromonas gingivalis lipid A phosphatase activity is critical for colonization and increasing the commensal load in the rabbit ligature model. Infect Immun 82:650-659.
18. Ogawa T, Asai Y, Makimura Y, Tamai R. 2007. Chemical structure and immunobiological activity of Porphyromonas gingivalis lipid A. Front Biosci 12:3795-3812.
19. Bainbridge B W, Darveau R P. 2001. Porphyromonas gingivalis lipopolysaccharide: an unusual pattern recognition receptor ligand for the innate host defense system. Acta Odontol Scand 59:131-8.
20. Poltorak A, He X, Smirnova I, Liu M Y, Van Huffel C, Du X, Birdwell D, Alejos E, Silva M, Galanos C, Freudenberg M, Ricciardi-Castagnoli P, Layton B, Beutler B. 1998. Defective LPS signaling in C3H/HeJ and C57BL/10ScCr mice: mutations in Tlr4 gene. Science 282:2085-8.
21. Hoshino K, Takeuchi O, Kawai T, Sanjo H, Ogawa T, Takeda Y, Takeda K, Akira S. 1999. Cutting edge: Toll-like receptor 4 (TLR4)-deficient mice are hyporesponsive to lipopolysaccharide: evidence for TLR4 as the Lps gene product. J Immunol 162:3749-52.
22. Shimazu R, Akashi S, Ogata H, Nagai Y, Fukudome K, Miyake K, Kimoto M. 1999. MD-2, a molecule that confers lipopolysaccharide responsiveness on Toll-like receptor 4. J Exp Med 189:1777-1782.
23. Coats S R, Pham T T, Bainbridge B W, Reife R A, Darveau R P. 2005. MD-2 mediates the ability of tetra-acylated and penta-acylated lipopolysaccharides to antagonize Escherichia coli lipopolysaccharide at the TLR4 signaling complex. J Immunol 175:4490-8.
24. Medzhitov R, Janeway C, Jr. 2000. The Toll receptor family and microbial recognition. Trends in Microbiology 8:452-456.
25. Raetz CR, Whitfield C. 2002. Lipopolysaccharide endotoxins. Annu Rev Biochem 71:635-700.
26. Jain S, Darveau R P. 2010. Contribution of Porphyromonas gingivalis lipopolysaccharide to periodontitis. Periodontol 2000 54:53-70.
27. Kumada H, Haishima Y, Watanabe K, Hasegawa C, Tsuchiya T, Tanamoto K, Umemoto T. 2008. Biological properties of the native and synthetic lipid A of Porphyromonas gingivalis lipopolysaccharide. Oral Microbiol Immunol 23:60-9.
28. Zhang Y, Gaekwad J, Wolfert M A, Boons G J. 2008. Synthetic tetra-acylated derivatives of lipid A from Porphyromonas gingivalis are antagonists of human TLR4. Org Biomol Chem 6:3371-81.
29. Coats S R, Jones J W, Do C T, Braham P H, Bainbridge B W, To T T, Goodlett D R, Ernst R K, Darveau R P. 2009. Human Toll-like receptor 4 responses to P. gingivalis are regulated by lipid A 1- and 4′-phosphatase activities. Cell Microbiol 11:1587-1599.
30. Al-Qutub M N, Braham P H, Karimi-Naser L M, Liu X, Genco C A, Darveau R P. 2006. Hemin-dependent modulation of the lipid A structure of Porphyromonas gingivalis lipopolysaccharide. Infect Immun 74:4474-4485.
31. Reife R A, Coats S R, Al-Qutub M, Dixon D M, Braham P A, Billharz R J, Howald W N, Darveau R P. 2006. Porphyromonas gingivalis lipopolysaccharide lipid A heterogeneity: differential activities of tetra- and penta-acylated lipid A structures on E-selectin expression and TLR4 recognition. Cell Microbiol 8:857-68.
32. Rangarajan M, Aduse-Opoku J, Hashim A, McPhail G, Luklinska Z, Haurat M F, Feldman M F, Curtis M A. 2017. LptO (PG0027) Is Required for Lipid A 1-Phosphatase Activity in Porphyromonas gingivalis W50. J Bacteriol 199.
33. Slocum C, Coats S R, Hua N, Kramer C, Papadopoulos G, Weinberg E O, Gudino C V, Hamilton J A, Darveau R P, Genco C A. 2014. Distinct lipid a moieties contribute to pathogen-induced site-specific vascular inflammation. PLoS Pathog 10:e1004215.
34. Trent M S, Pabich W, Raetz C R, Miller S I. 2001. A PhoP/PhoQ-induced Lipase (PagL) that catalyzes 3-O-deacylation of lipid A precursors in membranes of Salmonella typhimurium. J Biol Chem 276:9083-9092.
35. Reynolds C M, Ribeiro A A, McGrath S C, Cotter R J, Raetz C R H, Trent M S. 2006. An outer membrane enzyme encoded by Salmonella typhimurium IpxR that removes the 3′-acyloxyacyl moiety of lipid A. J Biol Chem 281:21974-21987.
36. Geurtsen J, Steeghs L, Hove J T, van der Ley P, Tommassen J. 2005. Dissemination of lipid A deacylases (pagL) among gram-negative bacteria: identification of active-site histidine and serine residues. J Biol Chem 280:8248-8259.
37. Chen Y-Y, Peng B, Yang Q, Glew M D, Veith P D, Cross K J, Goldie K N, Chen D, O'Brien-Simpson N, Dashper S G, Reynolds E C. 2011. The outer membrane protein LptO is essential for the O-deacylation of LPS and the co-ordinated secretion and attachment of A-LPS and CTD proteins in Porphyromonas gingivalis. Mol Microbiol 79:1380-1401.
38. Barth K, Remick D G, Genco C A. 2013. Disruption of immune regulation by microbial pathogens and resulting chronic inflammation. J Cell Physiol 228:1413-1422.
39. Palm E, Khalaf H, Bengtsson T. 2013. Porphyromonas gingivalis downregulates the immune response of fibroblasts. BMC Microbiol 13:155.
40. Hajishengallis G. 2011. Immune evasion strategies of Porphyromonas gingivalis. J Oral Biosci 53:233-240.
41. Duncan L, Yoshioka M, Chandad F, Grenier D. 2004. Loss of lipopolysaccharide receptor CD14 from the surface of human macrophage-like cells mediated by Porphyromonas gingivalis outer membrane vesicles. Microb Pathog 36:319-325.
42. Sugawara S, Nemoto E, Tada H, Miyake K, Imamura T, Takada H. 2000. Proteolysis of human monocyte CD14 by cysteine proteinases (gingipains) from Porphyromonas gingivalis leading to lipopolysaccharide hyporesponsiveness. J Immunol 165:411-418.
43. Goodman A L, McNulty N P, Zhao Y, Leip D, Mitra R D, Lozupone C A, Knight R, Gordon J I. 2009. Identifying genetic determinants needed to establish a human gut symbiont in its habitat. Cell Host Microbe 6:279-289.
44. Jain S, Coats S R, Chang A M, Darveau R P. 2013. A novel class of lipoprotein lipase-sensitive molecules mediates Toll-like receptor 2 activation by Porphyromonas gingivalis. Infect Immun 81:1277-1286.
45. Law C W, Alhamdoosh M, Su S, Smyth G K, Ritchie M E. 2016. RNA-seq analysis is easy as 1-2-3 with limma, Glimma and edgeR. F1000Res 5:1408.
46. Rutten L, Geurtsen J, Lambert W, Smolenaers J J M, Bonvin A M, de Haan A, van der Ley P, Egmond M R, Gros P, Tommassen J. 2006. Crystal structure and catalytic mechanism of the LPS 3-O-deacylase PagL from Pseudomonas aeruginosa. Proc Natl Acad Sci USA 103:7071-7076.
47. Rutten L, Mannie J-PBA, Stead C M, Raetz C R H, Reynolds C M, Bonvin A M J J, Tommassen J P, Egmond M R, Trent M S, Gros P. 2009. Active-site architecture and catalytic mechanism of the lipid A deacylase IpxR of Salmonella typhimurium. Proc Natl Acad Sci USA 106:1960-1964.
48. Yang J, Yan R, Roy A, Xu D, Poisson J, Zhang Y. 2015. The I-TASSER Suite: protein structure and function prediction. Nat Methods 12:7-8.
49. Walters S, Rodrigues P, Bélanger M, Whitlock J, Progulske-Fox A. 2009. Analysis of a band 7/MEC-2 family gene of Porphyromonas gingivalis. J Dent Res 88:34-38.
50. Song Y-H, Kozarov E V, Walters S M, Cao S L, Handfield M, Hillman J D, Progulske-Fox A. 2002. Genes of periodontopathogens expressed during human disease. Ann Periodontol 7:38-42.
51. Coats S R, Berezow A B, To T T, Jain S, Bainbridge B W, Banani K P, Darveau R P. 2011. The lipid A phosphate position determines differential host Toll-like receptor 4 responses to phylogenetically related symbiotic and pathogenic bacteria. Infect Immun 79:203-210.
52. Kawasaki K, Ernst R K, Miller S I. 2004. 3-0-deacylation of lipid A by PagL, a PhoP/PhoQ-regulated deacylase of Salmonella typhimurium, modulates signaling through Toll-like receptor 4. J Biol Chem 279:20044-8.
53. Ernst R K, Adams K N, Moskowitz S M, Kraig G M, Kawasaki K, Stead C M, Trent M S, Miller S I. 2006. The Pseudomonas aeruginosa lipid A deacylase: selection for expression and loss within the cystic fibrosis airway. J Bacteriol 188:191-201.
54. Kawahara K, Tsukano H, Watanabe H, Lindner B, Matsuura M. 2002. Modification of the structure and activity of lipid A in Yersinia pestis lipopolysaccharide by growth temperature. Infect Immun 70:4092-8.
55. Rebeil R, Ernst R K, Gowen B B, Miller S I, Hinnebusch B J. 2004. Variation in lipid A structure in the pathogenic yersiniae. Mol Microbiol 52:1363-73.
56. Reinés M, Llobet E, Dahlström K M, Pérez-Gutiérrez C, Llompart C M, Torrecabota

N, Salminen T A, Bengoechea J A. 2012. Deciphering the acylation pattern of Yersinia enterocolitica lipid A. PLoS Pathog 8:e1002978.
57. Montminy S W, Khan N, McGrath S, Walkowicz M J, Sharp F, Conlon J E, Fukase K, Kusumoto S, Sweet C, Miyake K, Akira S, Cotter R J, Goguen J D, Lien E. 2006. Virulence factors of Yersinia pestis are overcome by a strong lipopolysaccharide response. Nat Immunol 7:1066-1073.
58. Corcoran C P, Podkaminski D, Papenfort K, Urban J H, Hinton J C D, Vogel J. 2012. Superfolder GFP reporters validate diverse new mRNA targets of the classic porin regulator, MicF RNA. Mol Microbiol 84:428-445.
59. Zhang Y-F, Han K, Chandler C E, Tjaden B, Ernst R K, Lory S. 2017. Probing the sRNA regulatory landscape of P. aeruginosa: post-transcriptional control of determinants of pathogenicity and antibiotic susceptibility. Mol Microbiol 106:919-937.
60. Ogawa R, Yen H, Kawasaki K, Tobe T. 2018. Activation of IpxR gene through enterohaemorrhagic Escherichia coli virulence regulators mediates lipid A modification to attenuate innate immune response. Cell Microbiol 20.
61. Elhenawy W, Bording-Jorgensen M, Valguarnera E, Haurat M F, Wine E, Feldman M F. 2016. LPS Remodeling Triggers Formation of Outer Membrane Vesicles in Salmonella. mBio 7.
62. Gui M J, Dashper S G, Slakeski N, Chen Y-Y, Reynolds E C. 2016. Spheres of influence: Porphyromonas gingivalis outer membrane vesicles. Mol Oral Microbiol 31:365-378.
63. Xie H. 2015. Biogenesis and function of Porphyromonas gingivalis outer membrane vesicles. Future Microbiol 10:1517-1527.
64. Schromm A B, Brandenburg K, Loppnow H, Moran A P, Koch M H, Rietschel E T, Seydel U. 2000. Biological activities of lipopolysaccharides are determined by the shape of their lipid A portion. Eur J Biochem 267:2008-2013.
65. Haurat M F, Aduse-Opoku J, Rangarajan M, Dorobantu L, Gray M R, Curtis M A, Feldman M F. 2011. Selective Sorting of Cargo Proteins into Bacterial Membrane Vesicles. J Biol Chem 286:1269-1276.
66. Veith P D, Chen Y-Y, Gorasia D G, Chen D, Glew M D, O'Brien-Simpson N M, Cecil J D, Holden J A, Reynolds E C. 2014. Porphyromonas gingivalis outer membrane vesicles exclusively contain outer membrane and periplasmic proteins and carry a cargo enriched with virulence factors. J Proteome Res 13:2420-2432.
67. Gabarrini G, Heida R, van leperen N, Curtis M A, van Winkelhoff A J, van Dijl J M. 2018. Dropping anchor: attachment of peptidylarginine deiminase via A-LPS to secreted outer membrane vesicles of Porphyromonas gingivalis. Sci Rep 8:8949.
68. Gorasia D G, Veith P D, Chen D, Seers C A, Mitchell H A, Chen Y-Y, Glew M D, Dashper S G, Reynolds E C. 2015. Porphyromonas gingivalis Type IX Secretion Substrates Are Cleaved and Modified by a Sortase-Like Mechanism. PLoS Pathog 11:e1005152.
69. Veith P D, Glew M D, Gorasia D G, Reynolds E C. 2017. Type IX secretion: the generation of bacterial cell surface coatings involved in virulence, gliding motility and the degradation of complex biopolymers. Mol Microbiol 106:35-53.
70. Haurat M F, Elhenawy W, Feldman M F. 2015. Prokaryotic membrane vesicles: new insights on biogenesis and biological roles. Biol Chem 396:95-109.
71. Lappann M, Otto A, Becher D, Vogel U. 2013. Comparative proteome analysis of spontaneous outer membrane vesicles and purified outer membranes of Neisseria meningitidis. J Bacteriol 195:4425-4435.
72. Sidhu V K, Vorhölter F-J, Niehaus K, Watt S A. 2008. Analysis of outer membrane vesicle associated proteins isolated from the plant pathogenic bacterium Xanthomonas campestris pv. campestris. BMC Microbiol 8:87.
73. Galka F, Wai S N, Kusch H, Engelmann S, Hecker M, Schmeck B, Hippenstiel S, Uhlin B E, Steinert M. 2008. Proteomic characterization of the whole secretome of Legionella pneumophila and functional analysis of outer membrane vesicles. Infect Immun 76:1825-1836.
74. Kato S, Kowashi Y, Demuth D R. 2002. Outer membrane-like vesicles secreted by Actinobacillus actinomycetemcomitans are enriched in leukotoxin. Microb Pathog 32:1-13.
75. Elhenawy W, Debelyy M O, Feldman M F. 2014. Preferential packing of acidic glycosidases and proteases into Bacteroides outer membrane vesicles. MBio 5:e00909-00914.
76. Shi J, Zhao Y, Wang Y, Gao W, Ding J, Li P, Hu L, Shao F. 2014. Inflammatory caspases are innate immune receptors for intracellular LPS. Nature 514:187-192.
77. Hagar J A, Powell D A, Aachoui Y, Ernst R K, Miao E A. 2013. Cytoplasmic LPS activates caspase-11: implications in TLR4-independent endotoxic shock. Science 341:1250-1253.
78. Klein B A, Tenorio E L, Lazinski D W, Camilli A, Duncan M J, Hu L T. 2012. Identification of essential genes of the periodontal pathogen Porphyromonas gingivalis. BMC Genomics 13:578.

1. A plasmid expression vector comprising:
a portion of a tetC gene;
a Bacteroides promoter;
a multiple cloning site (MCS) downstream of the Bacteroides promoter; and
a bacterial gene inserted or cloned into the MCS;
wherein the plasmid expression vector is capable of expressing the bacterial gene in a host bacterial cell belonging to the Order Bacteroidales.

2. The plasmid expression vector of claim 1, further comprising one or more genes selected from a bla gene; a rep origin of replication; a repA origin of replication; an mbpA gene; an mbpB gene; an mbpC gene; a metR gene; and a tetQ gene.

3. The plasmid expression vector of claim 1, wherein the Bacteroides promoter is an ermF gene promoter.

4. The plasmid expression vector of claim 3, wherein the ermF gene promoter comprises at least 300 base pairs upstream of the ermF gene.

5. The plasmid expression vector of claim 1, wherein the MCS comprises restriction sites including StuI, XbaI, and SphI.

6. The plasmid expression vector of claim 1, wherein the bacterial gene is a P. gingivalis lipid A deacylase gene.

7. The plasmid expression vector of claim 1, wherein the P. gingivalis lipid A deacylase gene is PGN_1123.

8. The plasmid expression vector of claim 1, wherein the P. gingivalis lipid A deacylase gene comprises SEQ ID NO:1 or SEQ ID NO:2.

9. The plasmid expression vector of claim 1, wherein the plasmid expression vector comprises SEQ ID NO:5 or SEQ ID NO:6.

10. The plasmid expression vector of claim 1, wherein the plasmid expression vector is capable of expressing SEQ ID NO:3 or SEQ ID NO:4.

11. The plasmid expression vector of claim 1, wherein the host bacterial cell is a bacterial cell belonging to the genus Bacteroides.

12. (canceled)

13. A bacterial cell comprising the plasmid expression vector of claim 1.

14. The bacterial cell of claim 13, wherein the host bacterial cell comprising the plasmid expression vector is a P. gingivalis ΔPGN_1123 mutant containing the plasmid expression vector pSJ46, or containing plasmid expression vector+deacylase gene.

15. The bacterial cell of claim 13, wherein the bacterial cell comprising the plasmid expression vector is not a P. gingivalis cell.

16. The bacterial cell of claim 13, wherein the host bacterial cell comprising the plasmid expression vector is a bacterial cell belonging to the genus Bacteroides.

17. The bacterial cell of claim 13, wherein the host bacterial cell comprising the plasmid expression vector is a B. thetaiotaomicron cell.

18. The bacterial cell of claim 13, further comprising a population of said bacterial cells for use in bacterial cell culture system for screening inhibitors of lipid A deacylase, wherein each cell of the population of bacterial cells is a host bacterial cell.

19. (canceled)

20. (canceled)

21. A method of screening inhibitors of lipid A deacylase comprising:
contacting a population of bacterial cells comprising an expression vector of claim 1 with a candidate compound;
measuring TLR4 activity in the population of bacterial cells;
identifying the candidate compound as a deacylase inhibitor when TLR4 activity in the population of bacterial cells after contact with the candidate compound is increased as compared to TLR4 activity in the population of bacterial cells before contact with the candidate compound.

22. The method of claim 21, wherein population of bacterial cells belong to the genus Bacteroides.

23. The method of claim 22, wherein the population of bacterial cells are B. thetaiotaomicron cells.
